(19) United States
(12) Patent Application Publication
(10) Pub. No.: US 2024/0199626 A1
(43) Pub. Date: Jun. 20, 2024

(54) COMPOUNDS WITH IMPROVED CARDIAC SAFETY FOR THE TREATMENT OF CANCER AND NEURODEGENERATIVE DISORDERS

(71) Applicants: Oregon Health & Science University, Portland, OR (US); The Board of Trustees of the Leland Stanford Junior University, Stanford, CA (US)

(72) Inventors: Sanjay Malhotra, Portland, OR (US); Mallesh Pandrala, Portland, OR (US); Dhanir Tailor, Portland, OR (US); Arne A.N. Bruyneel, Stanford, CA (US); Mark Mercola, Stanford, CA (US); Anna P. Hnatiuk Hnatiuk, Stanford, CA (US)

(73) Assignees: Oregon Health & Science University, Portland, OR (US); The Board of Trustees of the Leland Stanford Junior University, Stanford, CA (US)

(21) Appl. No.: 18/556,081

(22) PCT Filed: Apr. 19, 2022
(86) PCT No.: PCT/US22/25400

§ 371 Date: Oct. 18, 2023

Related U.S. Application Data
(60) Provisional application No. 63/176,774, filed on Apr. 19, 2021.

(51) Int. Cl. C07D 487/04; A61K 31/496; A61K 31/5025; A61K 31/506; A61K 31/55; C07D 233/90

(52) U.S. Cl. CPC C07D 487/04; A61K 31/496; A61K 31/5025; A61K 31/506; A61K 31/55; C07D 233/90

(57) ABSTRACT

Provided herein are compounds, pharmaceutical formulations, and methods for treatment of cancer, particularly including chronic myeloid leukemias, and neurodegenerative disorders in a subject.

### CROSS-REFERENCE TO RELATED APPLICATIONS

This is the 371 National Phase of International Application No. PCT/US22/25400, filed Apr. 19, 2022, which claims priority to and the benefit of the earlier filing of U.S. Provisional Application No. 63/176,774, filed Apr. 19, 2021, which is incorporated by reference herein in its entirety.

### FIELD OF THE INVENTION

The present disclosure concerns new compounds, pharmaceutical formulations, and methods of treatment for cancer, particularly including chronic myeloid leukemia, and neurodegenerative disorders with greater cardiac safety.

### BACKGROUND OF THE INVENTION

Chronic myeloid leukemia (CML) is a myeloproliferative neoplasm that accounts for approximately 15% of newly diagnosed leukemia cases in adults and an estimated 61,090 new leukemia cases will be diagnosed in the USA in 2021.¹ The fusion protein product of the Philadelphia chromosome (Ph), BCR-ABL,²⁻⁶ is associated with CML and a subset acute lymphoblastic leukemia (Ph+ALL), thus, development of TKIs targeting the BCR-ABL oncogene constitute an effective approach to treating CML and/or ALL. For example, the kinase inhibitor imatinib (Gleevec, ST1571) is a first-line drug for patients diagnosed with CML, which inhibits the activity of the BCR-ABL kinase protein. The clinical success of imatinib paved the way to consider kinases as druggable targets.⁷⁻¹⁰ However, despite its durable initial response in most of the CML patients, imatinib fails in up to 40% patients due to the intolerance of the dose and drug resistance. Mutations within the kinase domain of BCR-ABL constitute the most frequent mechanism of drug resistance,¹¹⁻¹⁴ as it causes ineffective inhibitor binding with the target.¹⁵ To date, over 100 different point mutations have been identified in CML patients. To deal with these mutations, second and third generation inhibitors were discovered. From second generation: the imatinib family member nilotinib (Tasigna; AMN107), the multitargeted kinase inhibitor dasatinib (SPRYCEL®; BMS354825) and bosutinib (BOSULIF®; SKI-606) were approved for second-line use.^{13, 16-17} The second generation inhibitors demonstrated superior potency over imatinib, however, none of them have inhibited all of the imatinib-resistant mutations¹⁸⁻²⁰ in particular the T315I “gatekeeper” mutation (replacement of threonine by isoleucine at 315 position in the ABL1 kinase domain). The T315I gatekeeper mutations are reported in at least 20% of the CML patients.^{15, 21-22} When threonine is mutated to isoleucine in position 315, the bulkier isoleucine side chain extends into the enzyme active site, which causes steric hindrance preventing ATP-competitive inhibitors from binding the ATP binding pocket, consequently the first and the second-generation inhibitors are ineffective against the T315I mutations.^{17, 23-24} Furthermore, these inhibitors have shown adverse side effects on patients. Notably, increased risk of accumulated vascular events on the therapy for nilotinib,²⁵ pulmonary hypertension and myelosuppression for dasatinib, and increased ALT and AST levels for bosutinib are some of the adverse effects associated with these inhibitors.^{13,26} Several approaches have been demonstrated to address the T315I mutations; however, clinical development of these studies have been halted due to toxicity concerns.^{21,27} Nevertheless, a third generation multi-kinase inhibitor ponatinib has been approved in 2012 by the FDA with a broad label as a second-line treatment option for the patients with CML and Ph+ALL.²⁸ Ponatinib was shown to be most potent inhibitor among the TKIs that target BCR-ABL (FIG. 1). In addition, it has been demonstrated to have excellent activity against T315I mutant clones.²⁹⁻³¹ However, soon after its approval, ponatinib was found to have unaccepted levels of cardiovascular toxicity and its use was restricted to only those CML patients with T315I mutations. Notably, ponatinib is the only treatment option for the patients with the T315I mutation since the first and second generation inhibitors are ineffective.³² Ponatinib is one of the most cardiotoxic TKI in all the FDA approved TKIs.³³ Presumably because it concurrently inhibits multiple kinase and possibly other proteins involved in maintaining the function and integrity of the cardiovascular system.³⁴ Off-target effects caused by its binding to proteins with similar ATP pockets to that in BCR-ABL³⁵ result in adverse toxic complications and hence account for the use restriction.^{34, 36} Therefore, the development of a new TKI which works against the T315I mutation with improved safety to meet clinical needs is warranted. Several potential approaches have been reported to address the challenges associated with ponatinib. In most of the studies, the new inhibitor showed similar efficacies as ponatinib on native protein kinase but did not show much effect on the mutant T315I protein kinase.³⁷⁻³⁸

### SUMMARY OF THE INVENTION

A first embodiment provides a compound of Formula (I):

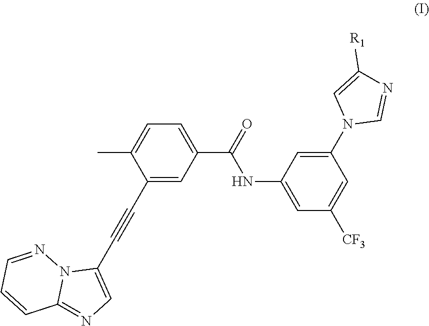

wherein R₁ is selected from the group of H, C₂-C₆ alkyl, C₃-C₆ cycloalkyl, and —CH₂-C₃-C₆ cycloalkyl; or a pharmaceutically acceptable salt thereof.

A second embodiment herein provides the compound of Formula (II), 4-methyl-3-((1-methyl-1H-imidazol-4-yl)ethynyl)-N-(4-((4-methylpiperazin-1-yl)methyl)-3-(trifluoromethyl)phenyl)benzamide, having the structure:

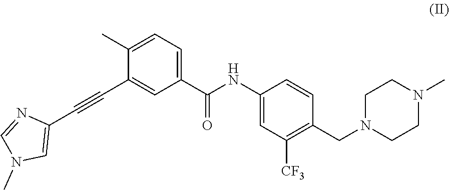 [SMILES: Cc1ccc(C(=O)Nc2ccc(CN3CCN(C)CC3)c(C(F)(F)F)c2)cc1C#Cc1cn(C)cn1]

or a pharmaceutically acceptable salt thereof.

Also provided herein and discussed below are pharmaceutical formulations comprising a pharmaceutically effective amount of a compound of Formula (I), or a pharmaceutically acceptable salt thereof, and/or a compound of Formula (II), or a pharmaceutically acceptable salt thereof.

### BRIEF DESCRIPTION OF THE MANY VIEWS OF THE DRAWINGS

FIG. 1A represents ponatinib binding interactions with native BCR-ABL protein.

FIG. 1B represents ponatinib binding interactions with BCR-ABL^{T315I} protein.

FIG. 1C represents a potential binding mode of inhibitors 33a and 36a with BCR-ABL protein.

FIG. 1D represents a potential binding mode of inhibitors 33a and 36a with BCR-ABL^{T315I} protein.

FIG. 2A represents binding interactions of ponatinib in superposition of both BCR-ABL and BCR-ABL^{T315I}.

FIG. 2B represents binding interactions of inhibitors 33a and 36a in superposition of both BCR-ABL and BCR-ABL^{T315I}.

FIG. 3A provides a graph of representative dose responses of Ponatinib, 33a, and 36a to assess relative cell viability in CML tumor cell line K562 cells.

FIG. 3B provides a graph comparing representative dose responses of Ponatinib, 33a, and 36a to assess relative cell viability in the same CML tumor cell line carrying the T315I ‘gatekeeper’ mutation (K562-T315I).

FIG. 3C provides a graph comparing representative dose responses of Ponatinib, 33a, 36a and control for angiogenesis by measuring the number of loops that form in Human Microvascular Endothelial cell cultures.

FIG. 3D provides a graph comparing representative dose responses of Ponatinib, 33a, 36a, and vehicle control (DMSO) on contractility (peak contraction amplitude) of cardiomyocytes (hiPSC-CMs, 15S1-WT cell line).

FIG. 3E provides a graph comparing representative dose responses of Ponatinib, 33a, 36a, and vehicle control (DMSO) on contractility (peak contraction amplitude) of cardiomyocytes (hiPSC-CMs, 273-WT cell line).

FIG. 4A presents a schematic representation of pharmacokinetic (PK) studies in mice for Ponatinib, 33a and 36a.

FIG. 4B presents a table of PK parameters for Ponatinib, 33a and 36a: Cmax, t-max and t1/2.

FIG. 4C presents a schematic representation of toxicity studies in mice over 30 days of compound treatment in increasing dose range up to maximum dose of 60 mg/kg.

FIG. 4D presents a Kaplan-Meier survival curve of the mice treated over 30 days with Vehicle, Ponatinib, 36a, and 33a.

FIG. 4E presents a schematic representation of xenograft studies in mice followed for 3 weeks of treatment with 30 mg/kg of Ponatinib, 67, and 84.

FIG. 4F presents a bar graph of comparative mouse weights after 3 weeks of treatment. FIG. 4G presents comparative excised tumors from treated mice.

FIG. 4H presents a bar graph representing comparative tumor weights in treated mice.

FIG. 4I presents a bar graph representing comparative troponin serum levels in treated mice.

### DETAILED DESCRIPTION OF THE INVENTION

Another embodiment provides a compound of Formula (I):

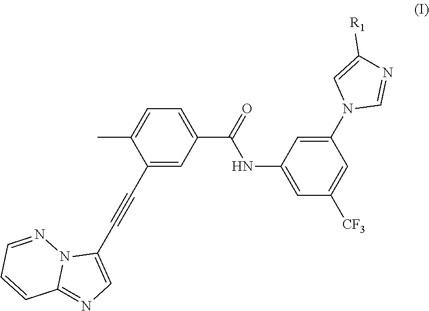

wherein R₁ is selected from the group of H, C₂-C₄ alkyl, cyclopropyl, and —CH₂-cyclopropyl; or a pharmaceutically acceptable salt thereof.

A further embodiment provides a compound of Formula (I), wherein R₁ is selected from the group of H, ethyl, n-propyl, isopropyl and cyclopropyl; or a pharmaceutically acceptable salt thereof.

A further embodiment provides a compound of Formula (I), wherein R₁ is selected from the group of H, ethyl, isopropyl and cyclopropyl; or a pharmaceutically acceptable salt thereof.

A further embodiment provides a compound of Formula (I), above, or a pharmaceutically acceptable salt thereof, wherein R₁ is selected from the group of H, ethyl, and cyclopropyl.

Another embodiment provides a compound of Formula (I), above, or a pharmaceutically acceptable salt thereof, wherein R₁ is selected from the group of H and ethyl.

Another embodiment provides a compound of Formula (I), above, or a pharmaceutically acceptable salt thereof, wherein R₁ is selected from the group of H and isopropyl.

Another embodiment provides a compound of Formula (I), above, or a pharmaceutically acceptable salt thereof, wherein R₁ is selected from the group of H and cyclopropyl.

Also provided is a method of treatment of chronic myeloid leukemia in a subject, the method comprising administering to the subject in need thereof a pharmaceutically effective amount of a compound of Formula (I), or a pharmaceutically acceptable salt thereof.

Also provided is a method of treatment of chronic myeloid leukemia in a subject, the method comprising administering to the subject in need thereof a pharmaceutically effective amount of a compound of Formula (II), or a pharmaceutically acceptable salt thereof.

Provided is a method of inhibiting the activity of the BCR-ABL kinase protein in a subject, the method comprising administering to the subject in need thereof a pharmaceutically effective amount of a compound of Formula (I), or a pharmaceutically acceptable salt thereof.

Provided is a method of inhibiting the activity of the BCR-ABL kinase protein in a subject, the method comprising administering to the subject in need thereof a pharmaceutically effective amount of a compound of Formula (II), or a pharmaceutically acceptable salt thereof.

Also provided is a method of treatment of chronic myeloid leukemia in a subject, the method comprising administering to the subject in need thereof:
a) a pharmaceutically effective amount of a compound of Formula (I), or a pharmaceutically acceptable salt thereof; and
b) a pharmaceutically effective amount of one or more agents selected from the group of ponatinib (ICLUSIG®), nilotinib (TASIGNA®), imatinib (GLEEVEC®), dasatinib (SPRYCELL®), bosutinib (BOSULIF®), rebastinib, and interferon alfa-2b; or a pharmaceutically acceptable salt thereof.

Also provided is a method of treatment of chronic myeloid leukemia in a subject, the method comprising administering to the subject in need thereof:
c) a pharmaceutically effective amount of a compound of Formula (II), or a pharmaceutically acceptable salt thereof; and
d) a pharmaceutically effective amount of one or more agents selected from the group of ponatinib (ICLUSIG®), nilotinib (TASIGNA®), imatinib (GLEEVEC®), dasatinib (SPRYCELL®), bosutinib (BOSULIF®), rebastinib, and interferon alfa-2b; or a pharmaceutically acceptable salt thereof.

Also provided is a method of treatment for chronic phase chronic myeloid leukemia in a subject, the method comprising administering to the subject in need thereof a pharmaceutically effective amount of a compound of Formula (I), or a pharmaceutically acceptable salt thereof.

Also provided is a method of treatment for chronic phase chronic myeloid leukemia in a subject, the method comprising administering to the subject in need thereof a pharmaceutically effective amount of a compound of Formula (II), or a pharmaceutically acceptable salt thereof.

Also provided is a method of treatment of chronic phase chronic myeloid leukemia in a subject, the method comprising administering to the subject in need thereof:
a) a pharmaceutically effective amount of a compound of Formula (I), or a pharmaceutically acceptable salt thereof; and
b) a pharmaceutically effective amount of one or more tyrosine kinase inhibiting agents selected from the group of ponatinib (ICLUSIG®), nilotinib (TASIGNA®), imatinib (GLEEVEC®), dasatinib (SPRYCELL®), bosutinib (BOSULIF®), and rebastinib; or a pharmaceutically acceptable salt thereof.

Also provided is a method of treatment of chronic phase chronic myeloid leukemia in a subject, the method comprising administering to the subject in need thereof:
a) a pharmaceutically effective amount of a compound of Formula (II), or a pharmaceutically acceptable salt thereof; and
b) a pharmaceutically effective amount of one or more tyrosine kinase inhibiting agents selected from the group of ponatinib (ICLUSIG®), nilotinib (TASIGNA®), imatinib (GLEEVEC®), dasatinib (SPRYCELL®), bosutinib (BOSULIF®), and rebastinib; or a pharmaceutically acceptable salt thereof.

Also provided is a method of treatment in a subject of chronic phase chronic myeloid leukemia with resistance or intolerance to at least one prior tyrosine kinase inhibitor, the method comprising administering to the subject in need thereof:
a) a pharmaceutically effective amount of a compound of Formula (I), or a pharmaceutically acceptable salt thereof; and
b) a pharmaceutically effective amount of ponatinib (ICLUSIG®); or a pharmaceutically acceptable salt thereof.

Also provided is a method of treatment in a subject of chronic phase chronic myeloid leukemia with resistance or intolerance to at least one prior tyrosine kinase inhibitor, the method comprising administering to the subject in need thereof:
a) a pharmaceutically effective amount of a compound of Formula (II), or a pharmaceutically acceptable salt thereof; and
b) a pharmaceutically effective amount of ponatinib (ICLUSIG®); or a pharmaceutically acceptable salt thereof.

Also provided is a method of treatment in a subject of chronic phase chronic myeloid leukemia with resistance or intolerance to at least two prior tyrosine kinase inhibitors, the method comprising administering to the subject in need thereof:
a) a pharmaceutically effective amount of a compound of Formula (I), or a pharmaceutically acceptable salt thereof; and
b) a pharmaceutically effective amount of ponatinib (ICLUSIG®); or a pharmaceutically acceptable salt thereof.

Also provided is a method of treatment in a subject of chronic phase chronic myeloid leukemia with resistance or intolerance to at least two prior tyrosine kinase inhibitors, the method comprising administering to the subject in need thereof:
a) a pharmaceutically effective amount of a compound of Formula (II), or a pharmaceutically acceptable salt thereof; and
b) a pharmaceutically effective amount of ponatinib (ICLUSIG®); or a pharmaceutically acceptable salt thereof.

Also provided is a method of treatment of accelerated phase chronic myeloid leukemia in a subject, the method comprising administering to the subject in need thereof:
a) a pharmaceutically effective amount of a compound of Formula (I), or a pharmaceutically acceptable salt thereof; and
b) a pharmaceutically effective amount of one or more tyrosine kinase inhibiting agents selected from the group of ponatinib (ICLUSIG®), nilotinib (TASIGNA®), imatinib (GLEEVEC®), dasatinib (SPRYCELL®), bosutinib (BOSULIF®), and rebastinib; or a pharmaceutically acceptable salt thereof.

Also provided is a method of treatment of accelerated phase chronic myeloid leukemia in a subject, the method comprising administering to the subject in need thereof:
a) a pharmaceutically effective amount of a compound of Formula (II), or a pharmaceutically acceptable salt thereof; and
b) a pharmaceutically effective amount of one or more tyrosine kinase inhibiting agents selected from the group of ponatinib (ICLUSIG®), nilotinib (TASIGNA®), imatinib (GLEEVEC®), dasatinib (SPRYCELL®), bosutinib (BOSULIF®), and rebastinib; or a pharmaceutically acceptable salt thereof.

Another embodiment provides a method of treatment of blast phase chronic myeloid leukemia in a subject, the method comprising administering to the subject in need thereof:
a) a pharmaceutically effective amount of a compound of Formula (I), or a pharmaceutically acceptable salt thereof; and
b) a pharmaceutically effective amount of one or more tyrosine kinase inhibiting agents selected from the group of ponatinib (ICLUSIG®), nilotinib (TASIGNA®), imatinib (GLEEVEC®), dasatinib (SPRYCELL®), bosutinib (BOSULIF®), and rebastinib; or a pharmaceutically acceptable salt thereof.

Another embodiment provides a method of treatment of blast phase chronic myeloid leukemia in a subject, the method comprising administering to the subject in need thereof:
a) a pharmaceutically effective amount of a compound of Formula (II), or a pharmaceutically acceptable salt thereof; and
b) a pharmaceutically effective amount of one or more tyrosine kinase inhibiting agents selected from the group of ponatinib (ICLUSIG®), nilotinib (TASIGNA®), imatinib (GLEEVEC®), dasatinib (SPRYCELL®), bosutinib (BOSULIF®), and rebastinib; or a pharmaceutically acceptable salt thereof.

Also provided is a method of treatment of chronic myeloid leukemia with a T315I mutation in a subject, the method comprising administering to the subject in need thereof:
a) a pharmaceutically effective amount of a compound of Formula (I), or a pharmaceutically acceptable salt thereof; and
b) a pharmaceutically effective amount of omacetaxine (SYNRIBO®); or a pharmaceutically acceptable salt thereof.

Also provided is a method of treatment of chronic myeloid leukemia with a T315I mutation in a subject, the method comprising administering to the subject in need thereof:
a) a pharmaceutically effective amount of a compound of Formula (II), or a pharmaceutically acceptable salt thereof; and
b) a pharmaceutically effective amount of omacetaxine (SYNRIBO®); or a pharmaceutically acceptable salt thereof.

Also provided is a method of treatment of Philadelphia chromosome positive chronic myeloid leukemia in a subject, the method comprising administering to the subject in need thereof:
a) a pharmaceutically effective amount of a compound of Formula (I), or a pharmaceutically acceptable salt thereof; and
b) a pharmaceutically effective amount of nilotinib (TASIGNA®); or a pharmaceutically acceptable salt thereof.

Also provided is a method of treatment of Philadelphia chromosome positive chronic myeloid leukemia in a subject, the method comprising administering to the subject in need thereof:
a) a pharmaceutically effective amount of a compound of Formula (II), or a pharmaceutically acceptable salt thereof; and
b) a pharmaceutically effective amount of nilotinib (TASIGNA®); or a pharmaceutically acceptable salt thereof.

Also provided is a method of treatment in a subject of chronic myeloid leukemia that is resistant or intolerant to prior tyrosine-kinase inhibitor (TKI) therapy, the method comprising administering to the subject in need thereof a pharmaceutically effective amount of a compound of Formula (I), or a pharmaceutically acceptable salt thereof.

Also provided is a method of treatment in a subject of chronic myeloid leukemia that is resistant or intolerant to prior tyrosine-kinase inhibitor (TKI) therapy, the method comprising administering to the subject in need thereof a pharmaceutically effective amount of a compound of Formula (II), or a pharmaceutically acceptable salt thereof.

Another embodiment provides a method of treating a neurodegenerative condition in a subject, the method comprising administering to the subject in need thereof a pharmaceutically effective amount of a compound of Formula (I), or a pharmaceutically acceptable salt thereof.

Another embodiment provides a method of treating a neurodegenerative condition in a subject, the method comprising administering to the subject in need thereof a pharmaceutically effective amount of a compound of Formula (II), or a pharmaceutically acceptable salt thereof.

The neurodegenerative disease of the methods above can be selected from the group of Parkinson's Disease, Alzheimer's Disease, Down's syndrome, frontotemporal dementia, progressive supranuclear palsy, Pick's disease, Niemann-Pick disease, Parkinson's disease, Huntington's disease (HD), dentatorubropallidoluysian atrophy, Kennedy's disease (also referred to as spinobulbar muscular atrophy), and spinocerebellar ataxia (e.g., type I, type 2, type 3 (also referred to as Machado-Joseph disease), type 6, type 7, and type 17)), fragile X (Rett's) syndrome, fragile XE mental retardation, Friedreich's ataxia, myotonic dystrophy, spinocerebellar ataxia type 8, and spinocerebellar ataxia type 12, Alexander disease, Alper's disease, amyotrophic lateral sclerosis, ataxia telangiectasia, Batten disease (also referred to as Spielmeyer-Vogt-Sjogren-Batten disease), Canavan disease, Cockayne syndrome, corticobasal degeneration, Creutzfeldt-Jakob disease, ischemia stroke, Krabbe disease, Lewy body dementia, multiple sclerosis, multiple system atrophy, Pelizaeus-Merzbacher disease, Pick's disease, primary lateral sclerosis, Adult Refsums Disease (ARD), Sandhoff disease, Schilder's disease, spinal cord injury, spinal muscular atrophy, Steele-Richardson-Olszewski disease, and Tabes dorsalis.

In some embodiments, the neurodegenerative condition is associated with, characterized by, or implicated by a mitochondrial dysfunction. Such neurodegenerative conditions associated with a mitochondrial dysfunction include, but are not limited to, Friedrich's ataxia, amyotrophic lateral sclerosis (ALS), mitochondrial myopathy, encephalopathy, lactacidosis, stroke (MELAS), myoclonic epilepsy with ragged red fibers (MERFF), epilepsy, Parkinson's disease, Alzheimer's disease, and Huntington's Disease.

Another embodiment provides a pharmaceutical composition comprising a pharmaceutically effective amount of a compound of Formula (I), or a pharmaceutically acceptable salt thereof, and a pharmaceutically useful carrier or excipient.

Another embodiment provides a pharmaceutical composition comprising a pharmaceutically effective amount of 4-methyl-3-((1-methyl-1H-imidazol-4-yl)ethynyl)-N-(4-((4-methylpiperazin-1-yl) methyl)-3-(trifluoromethyl) phenyl)benzamide (Formula (II)), or a pharmaceutically acceptable salt thereof, and a pharmaceutically useful carrier or excipient.

A further embodiment provides the use of a compound of Formula (I), or a pharmaceutically acceptable salt thereof, in the preparation of a medicament. It is understood that included herein are separate methods for preparation of a medicament for each of the subgeneric groups and individual compounds described herein within Formula (I), or a pharmaceutically acceptable salt thereof.

A still further embodiment provides the use of a compound of Formula (II), or a pharmaceutically acceptable salt thereof, in the preparation of a medicament.

It is understood that, in the methods herein concerning a combination therapy of a compound of Formula (I) or Formula (II), or a pharmaceutically acceptable salt thereof, with an additional agent, such as ponatinib (ICLUSIG®), nilotinib (TASIGNA®), imatinib (GLEEVEC®), dasatinib (SPRYCELL®), bosutinib (BOSULIF®), rebastinib, interferon alfa-2b, or omacetaxine (SYNRIBO®), the additional agents may be administered as determined by a medical professional based on the condition and the known and approved dosages and regimens for the additional agent(s) in question.

For example, tyrosine kinase inhibitor ponatinib (ICLUSIG®) may be administered at a daily dosage of from about 5 mg to about 60 mg. In some embodiments, ponatinib is administered once daily. In some embodiments, ponatinib is administered at individual daily doses of 10 mg, 15 mg, 30 mg, and 45 mg.

The agent nilotinib (TASIGNA®) may be administered at a daily dose of from about 50 mg to about 500 mg. Daily doses of 50 mg, 100 mg, 150 mg, 200 mg, 250 mg, 300 mg, 350 mg, 400 mg, 450 mg, and 500 mg may be given as an individual daily dose or divided into two (bid) or more separate doses. In instances where the treatment is a secondary treatment after a subject has been resistant to or intolerant of a prior treatment, such as with imatinib, the dosing of nilotinib may be at a daily dose of about 400 mg in as administration or divided into two administrations (bid).

The tyrosine kinase inhibiting agent imatinib (GLEEVEC®) may be administered at a daily dosage of from about 50 mg to about 800 mg per day in single (qd) or divided doses. Daily doses determined by a medical professional may be selected from the group of about 50 mg, about 100 mg, about 200 mg, about 300 mg, about 400 mg, about 500 mg, about 600 mg, about 700 mg and about 800 mg.

The tyrosine kinase inhibiting agent dasatinib (SPRYCELL®) may be administered at a daily dose of from about 10 mg to about 160 mg. Daily doses determined by a medical professional may be selected from the group of about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, and about 160 mg. Such doses may be administered in single or divided daily doses.

Kinase inhibitor bosutinib (BOSULIF®) may be administered at daily doses of from about 50 mg to about 600 mg per day in single or divided doses. Daily doses determined by a medical professional may be selected from the group of about 100 mg, about 200 mg, about 300 mg, about 400 mg, about 500 mg, and about 600 mg.

Rebastinib may be administered in the methods herein in daily doses of from about 50 mg to about 400 mg.

Immunomodulating agent interferon alfa-2b may be administered at a weekly dosage of from about 1 million Units/m² to about 60 million Units/m² in two or three divided administrations. Protein synthesis inhibitor omacetaxine (SYNRIBO®) may be administered at 1.25 mg/m² administered subcutaneously twice daily at approximately 12 hour intervals for 7 consecutive days every 28 days, over a 28-day cycle.

### Definitions

The wavy line ( [symbol] ) in chemical structures indicates a bond through which the structure shown is bound to another chemical moiety or group.

The term “alkyl” refers to a straight or branched hydrocarbon. For example, an alkyl group can include those having 1 to 6 carbon atoms (i.e, C₁-C₆ alkyl), 1 to 4 carbon atoms (i.e., C₁-C₄ alkyl), or 1 to 3 carbon atoms (i.e., C₁-C₃ alkyl). Examples of suitable alkyl groups include, but are not limited to, methyl, ethyl, n-propyl, isopropyl (−CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, and the like.

The term “cycloalkyl” refers to a saturated ring having 3 to 6 carbon atoms as a monocycle, including cyclopropyl, cyclobutyl, cyclopentyl, and cyclohexyl groups.

The term “subject” refers to an animal, such as a mammal, that has been or will be the object of treatment, observation or experiment. The methods described herein may be useful in both human therapy and veterinary applications. In some embodiments, the subject is a mammal; in some embodiments the subject is human; and in some embodiments the subject is chosen from cats and dogs. “Subject in need thereof” or “human in need thereof” refers to a subject, such as a human, who may have or is suspected to have diseases or conditions that would benefit from certain treatment; for example treatment with a compound of Formula (I), Formula (II), or Formula (III), or a pharmaceutically acceptable salt or co-crystal thereof, as described herein. This includes a subject who may be determined to be at risk of or susceptible to such diseases or conditions, such that treatment would prevent the disease or condition from developing.

The terms “effective amount,” “therapeutically effective amount,” or “pharmaceutically effective amount” refer to an amount that is sufficient to effect treatment, as defined below, when administered to a subject (e.g., a mammal, such as a human) in need of such treatment. The therapeutically or pharmaceutically effective amount will vary depending upon the subject and disease condition being treated, the weight and age of the subject, the severity of the disease condition, the manner of administration and the like, which can readily be determined by one of ordinary skill in the art. For example, an “effective amount,” “therapeutically effective amount,” or a “pharmaceutically effective amount” of a compound of Formula (I), Formula (II), or Formula (III), or a pharmaceutically acceptable salt or co-crystal thereof, is an amount sufficient to treat a subject (e.g., a human) suffering an indication, or to ameliorate or alleviate the existing symptoms of the indication. For example, a therapeutically or pharmaceutically effective amount may be an amount sufficient to chronic myeloid leukemia in a human subject.

In some embodiments, an effective amount of a compound, such a compound of Formula (I) or Formula (II), or a pharmaceutically acceptable salt thereof, is an amount that ranges from about 50 ng/kg body weight to about 50 pg/kg body weight (e.g., from about 50 ng/kg body weight to about 40 pg/kg body weight, from about 30 ng/kg body weight to about 20 pg/kg body weight, from about 50 ng/kg body weight to about 10 pg/kg body weight, from about 50 ng/kg body weight to about 1 pg/kg body weight, from about 50 ng/kg body weight to about 800 ng/kg body weight, from about 50 ng/kg body weight to about 700 ng/kg body weight, from about 50 ng/kg body weight to about 600 ng/kg body weight, from about 50 ng/kg body weight to about 500 ng/kg body weight, from about 50 ng/kg body weight to about 400 ng/kg body weight, from about 60 ng/kg body weight to about 400 ng/kg body weight, from about 70 ng/kg body weight to about 300 ng/kg body weight, trom about 60 ng/kg body weight to about 100 ng/kg body weight, from about 65 ng/kg body weight to about 85 ng/kg body weight, from about 70 ng/kg body weight to about 90 ng/kg body weight, from about 200 ng/kg body weight to about 900 ng/kg body weight, from about 200 ng/kg body weight to about 800 ng/kg body weight, from about 200 ng/kg body weight to about 700 ng/kg body weight, from about 200 ng/kg body weight to about 600 ng/kg body weight, from about 200 ng/kg body weight to about 500 ng/kg body weight, from about 200 ng/kg body weight to about 400 ng/kg body weight, or from about 200 ng/kg body weight to about 300 ng/kg body weight).

In some embodiments, an effective amount of a compound is an amount that ranges from about 10 pg to about 100 mg, e.g., from about 10 pg to about 50 pg, from about 50 pg to about 150 pg, from about 150 pg to about 250 pg, from about 250 pg to about 500 pg, from about 500 pg to about 750 pg, from about 750 pg to about 1 ng, from about 1 ng to about 10 ng, from about 10 ng to about 50 ng, from about 50 ng to about 150 ng, from about 150 ng to about 250 ng, from about 250 ng to about 500 ng, from about 500 ng to about 750 ng, from about 750 ng to about 1 pg, from about 1 pg to about 10 pg, from about 10 pg to about 50 pg, from about 50 mg to about 150 gg, from about 150 gg to about 250 gg, from about 250 gg to about 500 gg, from about 500 gg to about 750 gg, from about 750 gg to about 1 g, from about 1 mg to about 50 mg, from about 1 mg to about 100 mg, or from about 50 mg to about 100 mg. The amount can be a single dose amount or can be a total daily amount. The total daily amount can range from 10 pg to 100 mg, or can range from 100 mg to about 500 mg, or can range from 500 mg to about 1000 mg.

In some embodiments, a single dose of a compound is administered. In other embodiments, multiple doses are administered. Where multiple doses are administered over a period of time, the compound can be administered twice daily (qid), daily (qd), every other day (qod), every third day, three times per week (tiw), or twice per week (biw) over a period of time. For example, a compound is administered qid, qd, qod, tiw, or biw over a period of from one day to about 2 years or more. For example, a compound is administered at any of the aforementioned frequencies for one week, two weeks, one month, two months, six months, one year, or two years, or more, depending on various factors.

The term “pharmaceutical composition” refers to a composition containing a pharmaceutically effective amount of one or more of the isotopic compounds described herein, or a pharmaceutically acceptable salt thereof, formulated with a pharmaceutically acceptable carrier, which can also include other additives, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein. Conventional procedures and ingredients for the selection and preparation of suitable formulations are described, for example, in Remington: The Science and Practice of Pharmacy, 21^{st} Ed., Gennaro, Ed., Lippencott Williams & Wilkins (2005) and in The United States Pharmacopeia: The National Formulary (USP 36 NF31), published in 2013. As used herein, “pharmaceutically acceptable excipient” is a pharmaceutically acceptable vehicle that includes, without limitation, any and all carriers, solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

The term “pharmaceutically acceptable carrier” refers to any ingredient in a pharmaceutical composition other than the disclosed pharmaceutically active or therapeutic compounds, including those of Formulas (I) and (II), or a pharmaceutically acceptable salt thereof (e.g., a carrier capable of suspending or dissolving the active isotopic compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The term “pharmaceutically acceptable salt” includes, for example, salts with inorganic acids and salts with an organic acid. Examples of salts may include hydrochloride, phosphate, diphosphate, hydrobromide, sulfate, sulfinate, nitrate, malate, maleate, fumarate, tartrate, succinate, citrate, acetate, lactate, methanesulfonate (mesylate), benzenesuflonate (besylate), p-toluenesulfonate (tosylate), 2-hydroxyethylsulfonate, benzoate, salicylate, stearate, and alkanoate (such as acetate, HOOC—(CH₂)ₙ—COOH where n is 0-4). In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts.

Also included for the compounds of Formula (I) and Formula (II) described herein are the pharmaceutically acceptable salts, pharmaceutically acceptable co-crystals, pharmaceutically acceptable esters, pharmaceutically acceptable solvates, hydrates, isomers (including optical isomers, racemates, or other mixtures thereof), tautomers, isotopes, polymorphs, and pharmaceutically acceptable prodrugs of such compounds. For the sake of brevity, the list of forms in the prior sentence may not be listed in all references to compounds herein, including those of Formula (I) and Formula (II), but each is understood to be disclosed and included herein, even if only pharmaceutically acceptable salts are included in a description applied anywhere herein, including in association with descriptions of chemical compounds, pharmaceutical compositions, methods of use/treatment, or other references.

The term “crystal forms” and related terms herein refer to the various crystalline modifications of a given substance, including, but not limited to, polymorphs, solvates, hydrates, co-crystals, and other molecular complexes, as well as salts, solvates of salts, hydrates of salts, other molecular complexes of salts, and polymorphs thereof. Crystal forms of a substance can be obtained by a number of methods, as known in the art. Such methods include, but are not limited to, melt recrystallization, melt cooling, solvent recrystallization, recrystallization in confined spaces such as, e.g., in nanopores or capillaries, recrystallization on surfaces or templates, such as, e.g., on polymers, recrystallization in the presence of additives, such as, e.g., co-crystal counter-molecules, desolvation, dehydration, rapid evaporation, rapid cooling, slow cooling, vapor diffusion, sublimation, grinding and solvent-drop grinding.

The descriptions herein set forth exemplary methods, parameters and the like. It should be recognized, however, that such descriptions are not intended as a limitation on the scope of the present disclosure but is instead provided as a description of exemplary embodiments.

Drawing from the knowledge discussed above, we envisioned that if a TKI is effective against both native and T315I mutant BCR-ABL, and highly cardiac-safe versus ponatinib, it would not only gain a broader scope of utilization but it would become a huge relief to the CML patients with T315I mutations. Given that imatinib and nilotinib are relatively cardiac-safe compared to ponatinib,^{26, 33} we hypothesized that it should be possible to discover a cardiac-safe BCR-ABL inhibitor by modifying the structure of the existing BCR-ABL inhibitors. We believe that H bond interactions between the TKIs and Met318 residue in BCR-ABL is essential for the TKIs to show efficacies against BCR-ABL. Therefore, using the core structure of each TKI that is responsible for H bond interaction with Met318, and study SAR around that core for efficacies and cardiac safety would yield therapeutics more broadly applicable in the clinic. We speculated that the new inhibitors would possibly maintain the H bond interactions with Met318, thus they could show similar efficacies as the parent TKI of the core structure. As a proof of concept, we combined our drug design paradigm with iPSC-CM models to predict the cardiotoxicities for the new analogues in the early stage. As expected, the newly designed inhibitors have exhibited similar efficacies as benchmark FDA drugs against the K-562 cell line, a BCR-ABL positive CML line. In addition, they have also shown excellent efficacies against K-562 cells expressing BCR-ABLT315I. Since the iPSC-CM cardiotoxicity assay is an integral part of our drug design, we identified cardiotoxic cores in the early stage and avoided using them in further studies. As a result, we finally identified cardiac-safe cores and studied SAR around the core for efficacies against both native and T315I mutant cell lines, while maintaining cardiac-safety. Extensive SAR studies led to the discovery of inhibitors 33a and 36a, which have significantly improved cardiac-safety over ponatinib, yet inhibited the kinase activity of BCR-ABL^{T315I}, and potently inhibited proliferation of the corresponding K-562 cell line.

### Molecular Design and Computational Studies

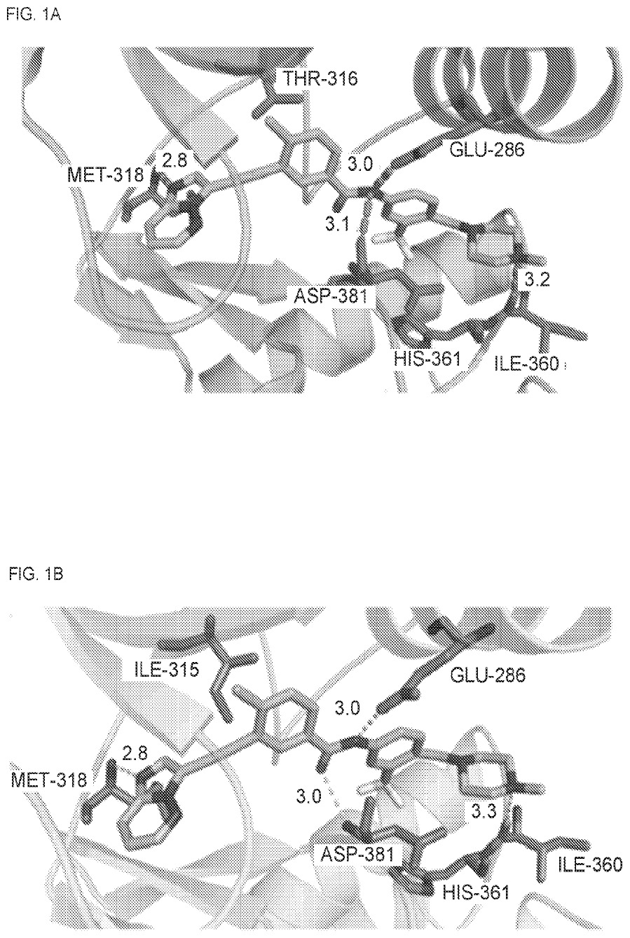

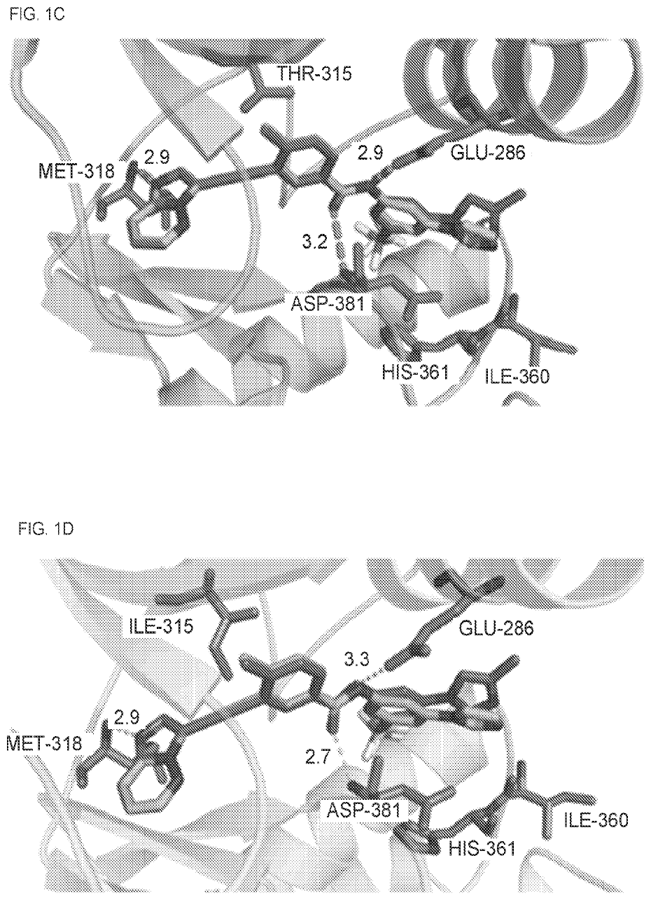

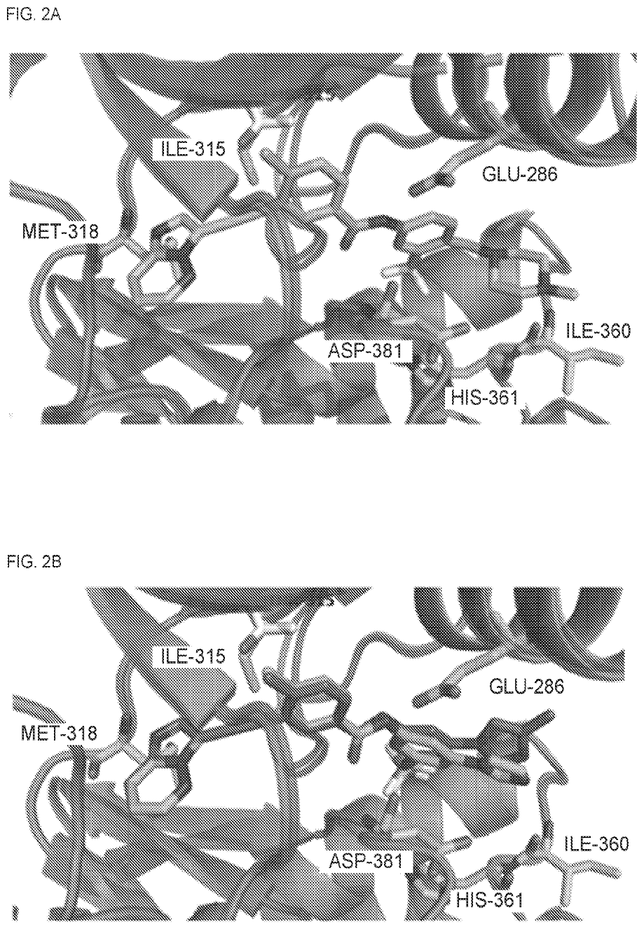

The FDA approved BCR-ABL inhibitors make H bond interactions with the backbone of Met318 in the hinge region in native BCR-ABL. In addition, inhibitors such as imatinib, dasatinib and nilotinib make a key hydrogen bond to the side chain of the gatekeeper residue Thr315.^{17,24,39} Formation of this hydrogen bond is critical for these inhibitors activity. Therefore, if the gatekeeper residue is mutated to isoleucine (mutation T315I), this hydrogen bond is lost. Steric clashes of the more bulky isoleucine residue blocks the inhibitor entry to the hydrophobic pocket, which can also cause loss of hydrogen bond interaction with Met318. As a result, they are inactive against T315I mutation. The steric clashes of isoleucine were also observed for bosutinib. Consequently, the only hydrogen bond that bosutinib makes with Met318 in native BCR-ABL is prohibited when threonine at the 315 position of BCR-ABL is mutated to isoleucine, and therefore it is inactive.⁴⁰ In contrast, ponatinib does not make H bond interactions with Thr315 in native BCR-ABL but makes a H bond interactions with Met318 with both native and T315I mutant BCR-ABL kinase (FIGS. 1A, 1B), so subsequently it inhibits both native BCR-ABL and BCR-ABLT315I kinases,²⁹ and emerged as a unique treatment option for patients with the T315I mutation.³² Based on these observations, we understood that a hydrogen bond between the inhibitor and Met318 is crucial in order to show activity on both native BCR-ABL and BCR-ABLT315I kinases. Therefore, we hypothesized that designing a hybrid molecule using core structures of the known BCRABL inhibitors, which make H bond interactions with Met318 and study their binding interactions with native BCR-ABL and BCR-ABLT315I protein would be an ideal first step. Using core structures of approved BCR-ABL inhibitors, several hybrid molecules were designed and computational studies were performed, to investigate the potential binding modes of the designed compounds. The computational studies revealed that the majority of the hybrids possessed the key hydrogen interaction with the backbone of Met318 in the hinge region in native BCR-ABL. Moreover, some of the hybrids showed hydrogen bond interactions with Met318 in BCR-ABLT315I. Particularly, the hybrids that were designed using a core structure from ponatinib (the core structure similar to 8), occupied the ATP-pocket of the BCR-ABL^{T315I} and showed a hydrogen bond interaction with the backbone of Met318. Furthermore, as shown in FIGS. 1C, 1D, the lead compounds 33a and 36a occupied the same binding region that ponatinib occupies in BCR-ABL^{T315I}, thus they have shown the same distance between the N atom of the Met318 residue and the N atom of imidazo[1,2-b]pyridazine moiety of inhibitors (FIGS. 1C, 1D). Moreover, the distance between the atoms of other key residues such as Glu286 and Asp381 and the atoms of the lead compounds, which could potentially interact with these residues by H bond were also similar to that observed for ponatinib with BCR-ABL^{T315I}. The superposition of both BCR-ABL and BCR-ABL^{T315I} kinase (FIGS. 2A, 2B) and the poses of lead compounds revealed that the ethyl linker in these inhibitors would skirt the mutated gatekeeper residue Ile315, as similar to ponatinib.⁴¹ Therefore, these compounds could possibly show similar efficacies as ponatinib in inhibiting BCR-ABL^{T315I}.

FIG. 1 provides representations of lead compounds binding interactions with native BCR-ABL and BCR-ABL^{T315I} protein. a) Ponatinib binding interactions with native BCR-ABL; b) Ponatinib binding interactions with BCR-ABL^{T315I} , Potential binding mode of inhibitors 33a and 36a with BCR-ABL (c) and BCR-ABL^{T315I} (d). PDB IDs for BCR-ABL and BCR-ABL^{T315I} are 3OXZ and 3IK3, respectively. The key residues, which will potentially make critical interactions with inhibitors, are shown in stick form and labeled. The distance between two atoms are indicated in yellow dashed lines and labeled in black.

FIG. 2 provides a comparison of binding interactions of (a) ponatinib with (b) inhibitors 33a and 36a in superposition of both BCR-ABL and BCR-ABL^{T315I} . PDB IDs for BCR-ABL and BCR-ABL^{T315I} are 3OXZ and 3IK3, respectively. The key residues, which will potentially make critical interactions with inhibitors, are shown in stick form.

### Chemistry

The compound 3a was obtained from a commercial source (Ark Pharma). The synthesis of 2-amino-N-(2-chloro-6-methylphenyl)thiazole-5-carboxamide based inhibitors 3 b-d is shown scheme-1. N-(2-chloro-6-methylphenyl)-2-((2-methylpyrimidin-4-yl)amino)thiazole-5-carboxamide 3b, was prepared according to the previously reported procedure for a similar analogue, 42 by the SNAr displacement of 4-chloro-2-methylpyrimidine with 2-amino-N-(2-chloro-6-methylphenyl)thiazole-5-carboxamide 1. Alternatively, 3 c-d were obtained by amide coupling in the presence of EDC.HCl and HOBt. The inhibitors 11 a-c were synthesized based on the tandem Sonogashira strategy using a previously reported procedure for similar analogues.⁴³ As illustrated in scheme 2, two general methods (A and B) were explored using either the 3-bromoimidazo[1,2-b]pyridazine 4 or methyl 3-iodo-4-methylbenzoate 6 as coupling agents in the first Sonogashira reaction. The first Sonogashira reaction was a straightforward reaction and occurred on both of the reagents 4 and 6, and the corresponding products were isolated in good yields. However, the final Sonogashira reaction employed in method B resulted in very low yields of the desired product, with debromination of 4 being the major impurity. Therefore, method A was used to synthesize 8. Hydrolysis of 8 using 1M LiOH solution afforded 9, which upon reaction with appropriate amines 10 a-c in standard amide coupling conditions, using EDC and HOBt, afforded the final compounds 11a-c. The synthetic scheme 2 could also facilitate the synthesis of inhibitor 15. However, it was synthesized using a convenient alternate route as out lined in scheme 3. An amide coupling of the readily available 3-iodo-4-methylbenzoic acid 12 with 3-bromo-5-(trifluoromethyl)aniline 13 in the presence of SOCI2 and DIPEA offered the intermediate 14. Subsequent Sonogashira coupling of 14 with 5 provided inhibitor 15. Inhibitors 19, 20 21 a-b and 24 were synthesized according to the synthetic route outlined in scheme 4. Starting material 3-ethynyl-4-methylbenzoic acid 16 was obtained from hydrolysis of methyl 3-ethynyl-4-methylbenzoate 7. Amide coupling of 17 with 16 afforded 19, which underwent Sonogashira coupling with 4-iodo-1-methyl-1H-imidazole to yield 20. Inhibitors 21 a-b were prepared similarly to 19 using 18 a-b instead of 16 as the carboxylic acid precursors. Inhibitor 24 was prepared from 17 via an amide coupling followed by a Sonogashira reaction, with appropriate starting materials.

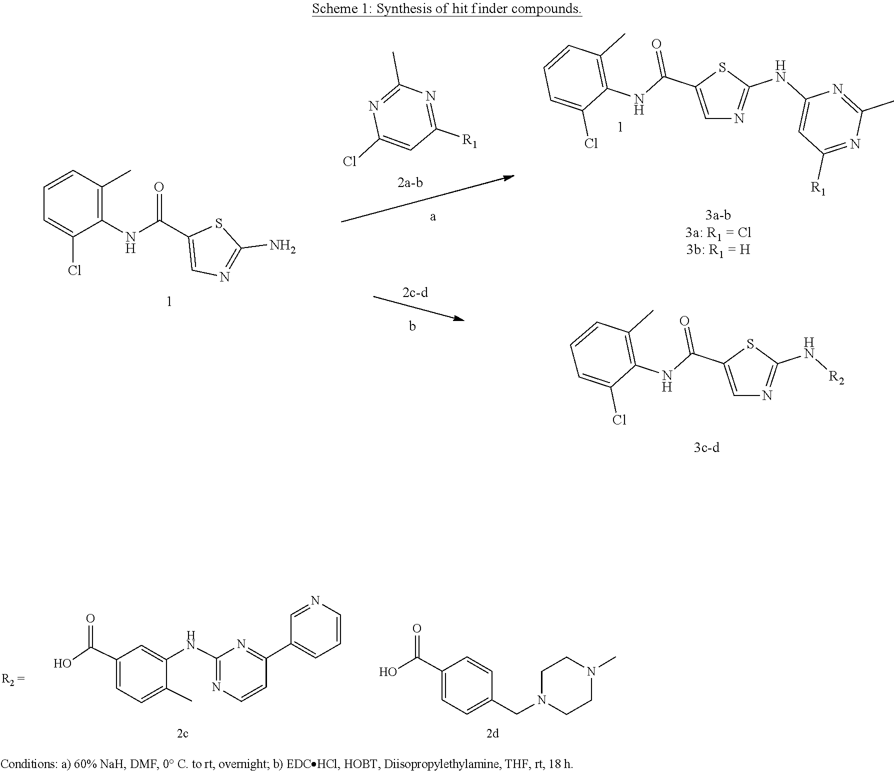

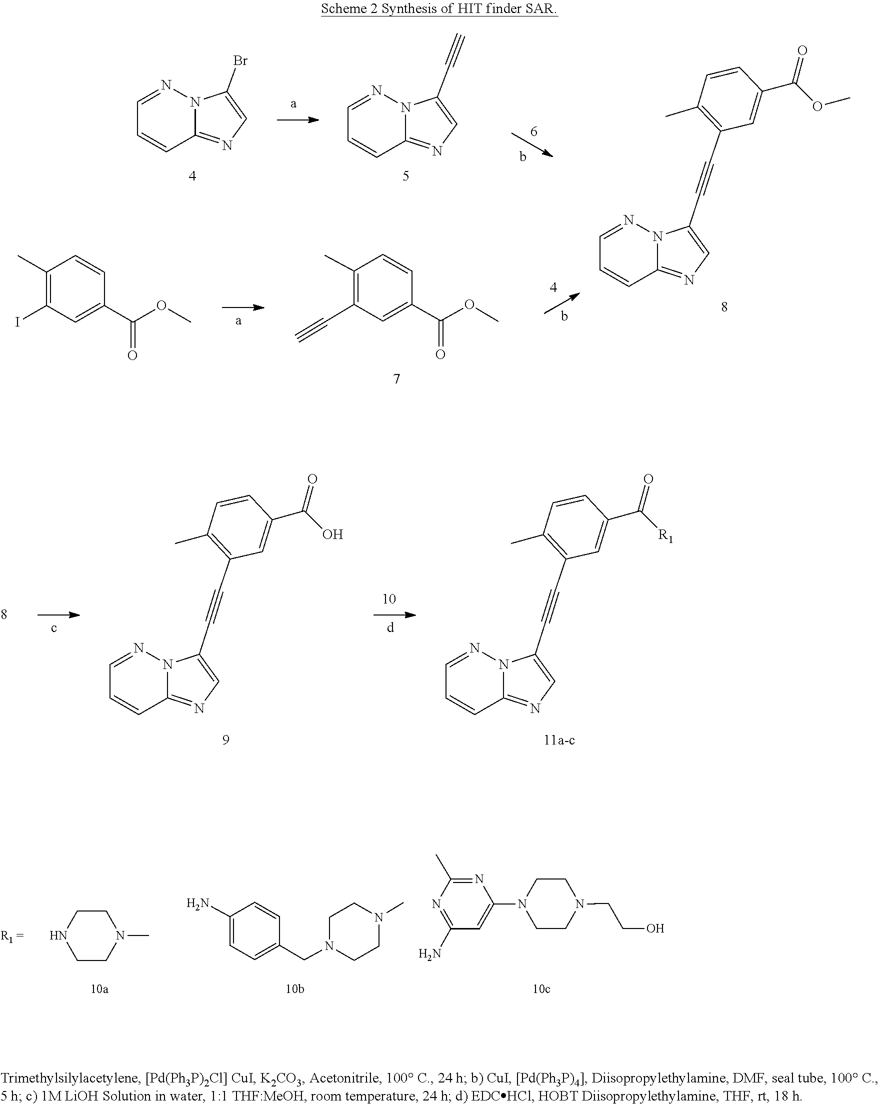

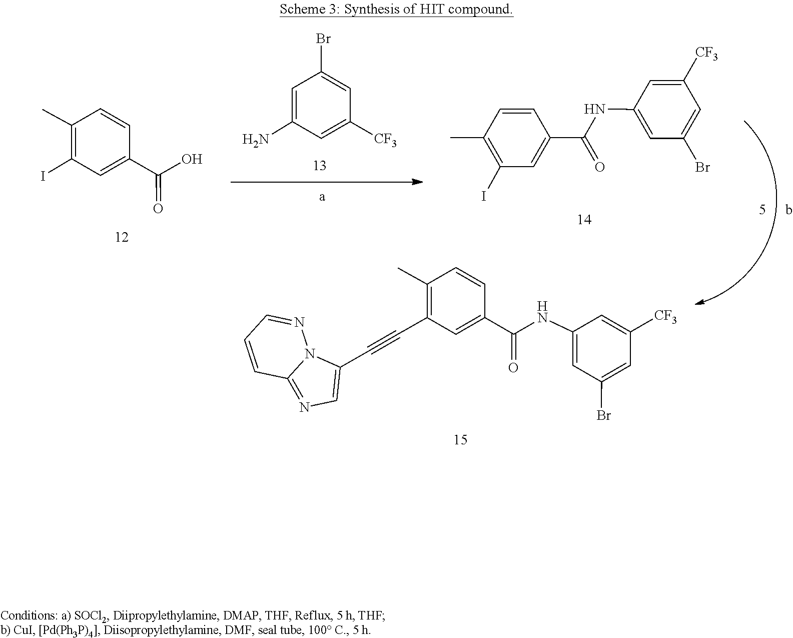 [SMILES: Cc1ccc(C(=O)O)cc1I ; Nc1cc(Br)cc(C(F)(F)F)c1 ; Cc1ccc(C(=O)Nc2cc(Br)cc(C(F)(F)F)c2)cc1I ; Cc1ccc(C(=O)Nc2cc(Br)cc(C(F)(F)F)c2)cc1C#Cc1cnc2cccnn12]

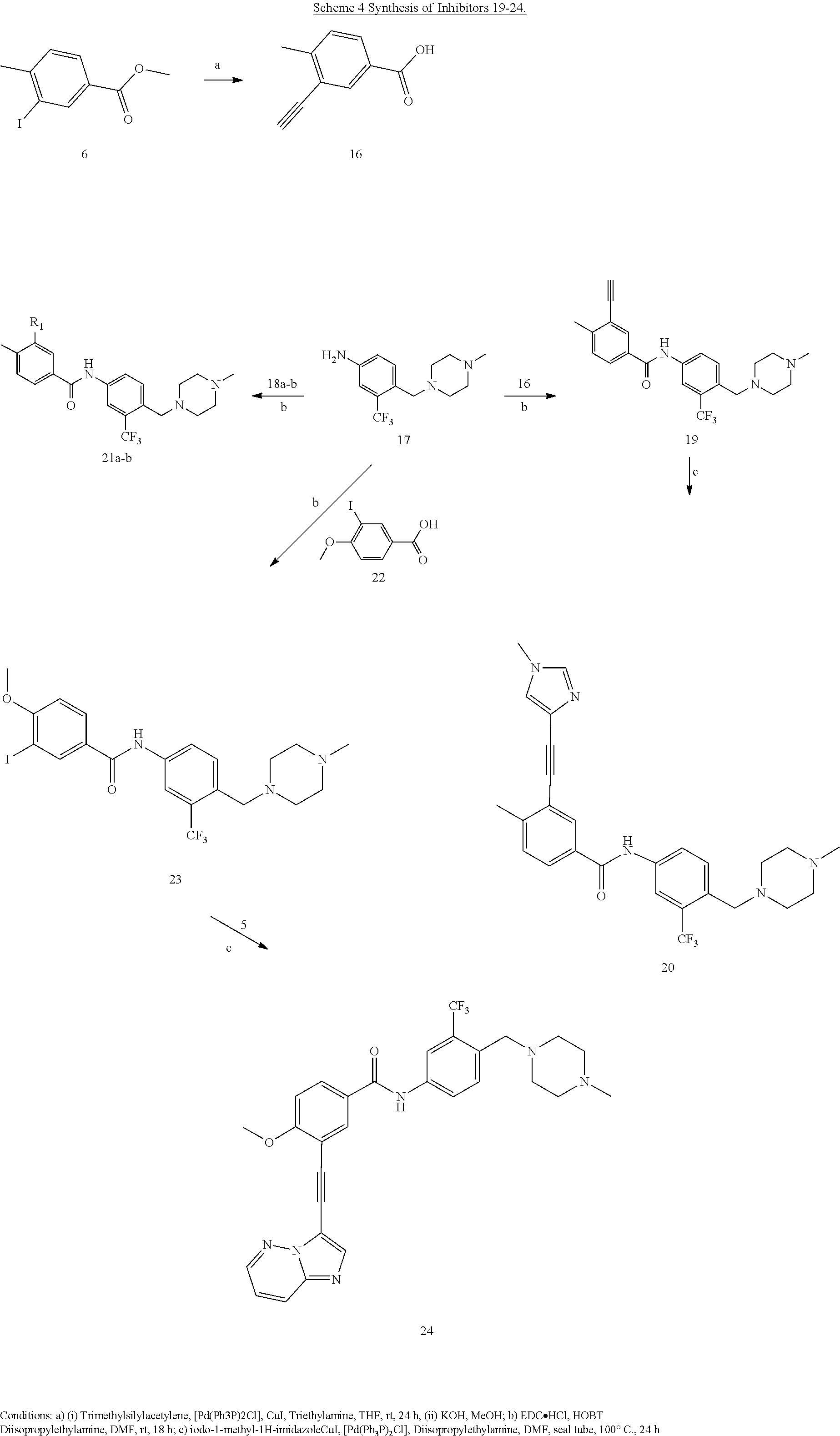

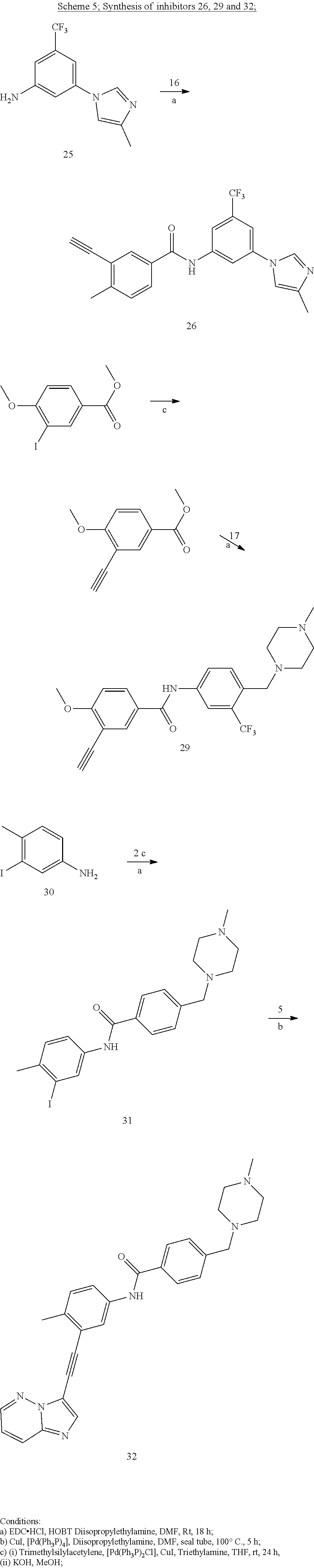

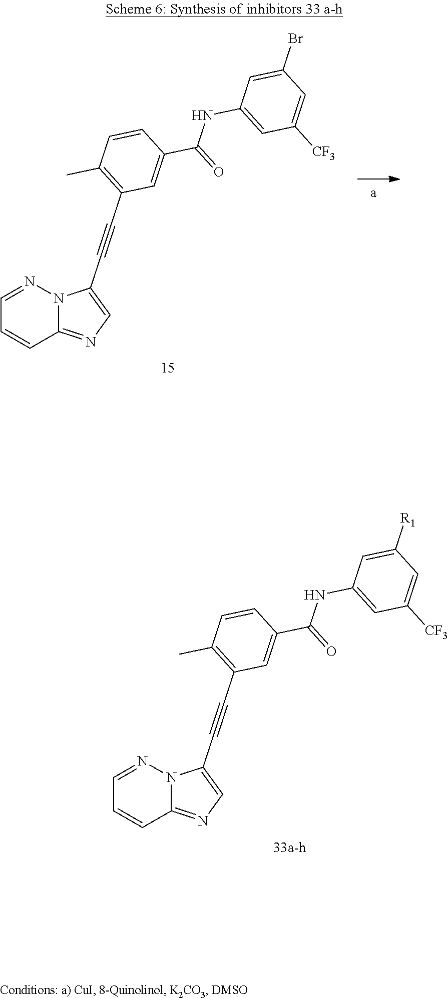

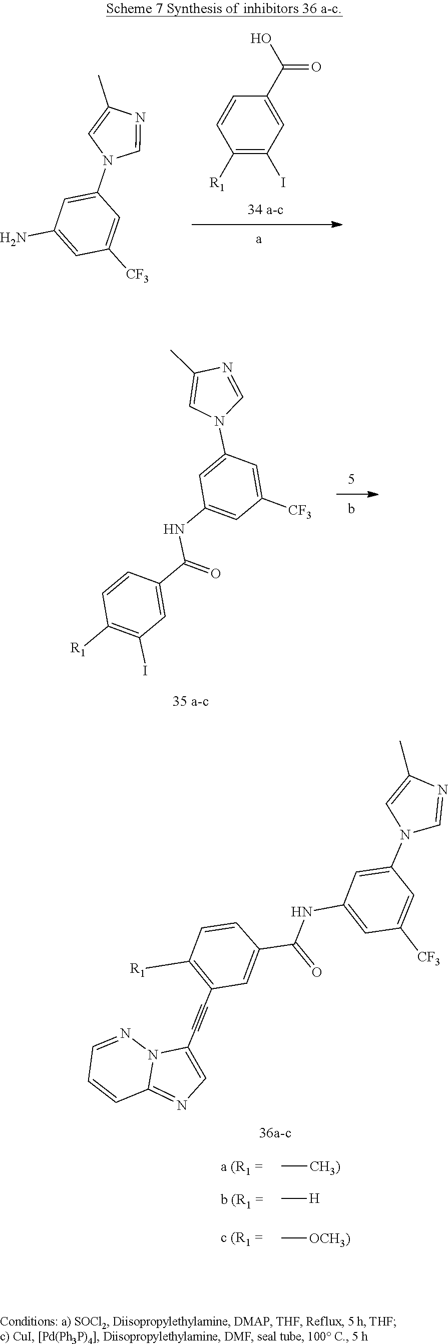

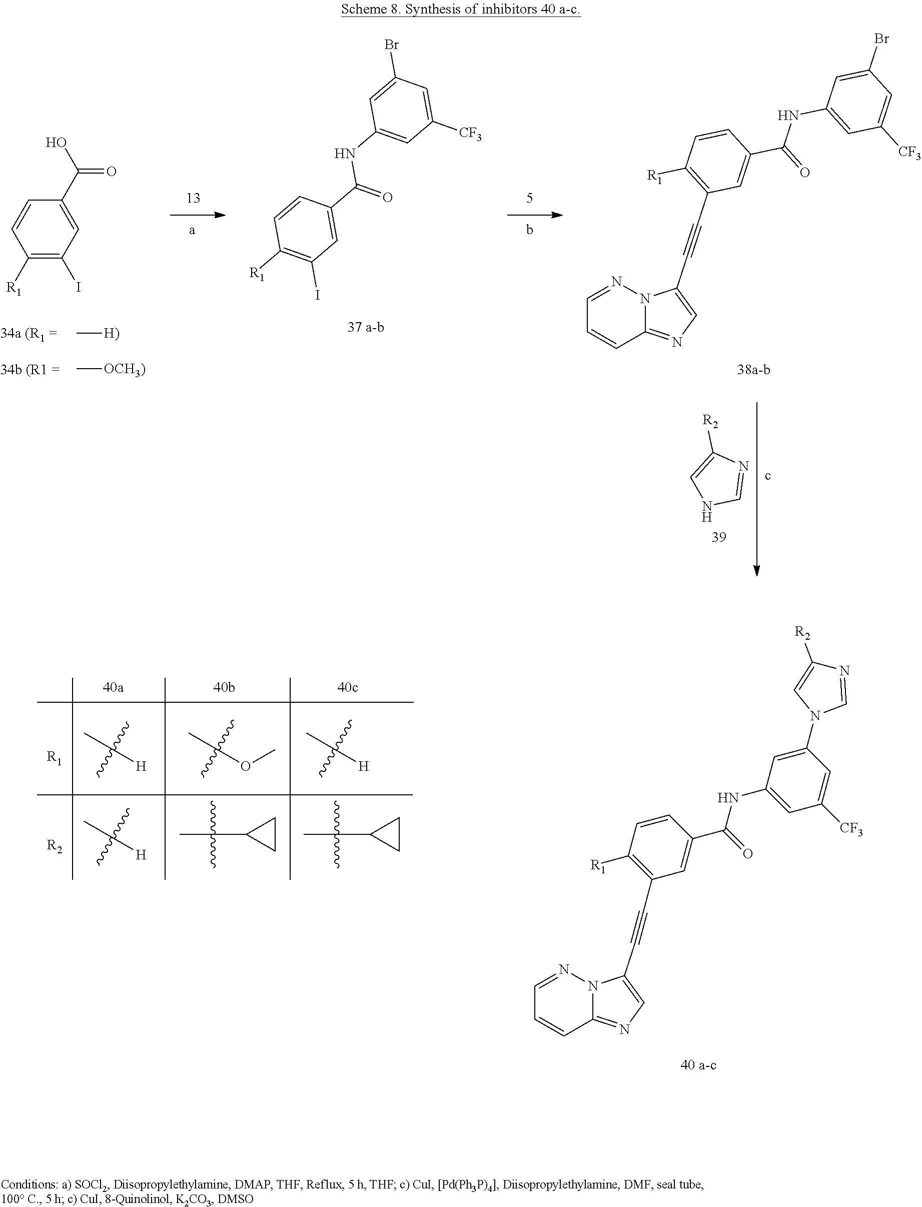

Synthesis of inhibitors 26, 29 and 32 were depicted in scheme 5. Inhibitor 26 was obtained by reacting 3-(4-methyl-1H-imidazol-1-yl)-5-(trifluoromethyl)aniline 25 with 16 using standard EDC-HOBt amide coupling conditions. Inhibitor 29 was prepared similar to 19, using the required starting materials for both the Sonogashira reactions. The structure of inhibitor 32 resembles 11b, however, the position of the amide group in 32, which was flipped over in between the two aryl groups, makes the difference in 32. It was prepared in two steps. In the initial step, amide condensation was performed between 3-iodo-4-methylaniline 30 and 2d to obtain intermediate 31, which was then reacted with 5 via Sonogashira reaction conditions to provide the inhibitor 32.

Scheme 6 illustrates the synthesis of inhibitors 33a-h compiled in Table 5. Briefly, a copper catalyzed N-arylation⁴⁴⁻⁴⁶ of imidazole or substituted imidazoles or methyl pyrrole or methyl piperazine with 15 yielded corresponding compounds 33a-h. Notably, the coupling reaction worked well for all of the substrates that were reported here, however, a slight decrease in isolated yields were observed for the inhibitors 33e and 33f, with pyrrole and methyl piperazine moieties, respectively.

The synthetic protocols for inhibitors 36a-c and 40a-c are outlined in scheme 7 and 8, respectively. They were prepared using the amide coupling and Sonogashira procedures outlined in scheme 5.

Results and discussion

While the H bond interactions between the inhibitor and Met318 residue in BCR-ABL play a crucial role in the activity, we initially selected core fragments of the FDA approved TKIs, which make H bond interactions with Met318 and designed hybrid molecules to identify a cardiac-safe ‘HIT’ molecule. The hybrids were evaluated for their kinase and cellular activities in vitro, against both BCR-ABL and BCR-ABL^{T315I} kinases and corresponding K-562 cell lines. Additionally, their cardiotoxicities were also evaluated by probing contractility and voltage transients in iPSC-CMs to help guide template selection. The inhibitors such as imatinib, dasatinib and ponatinib were used as controls to validate the screening conditions.

As shown in Table 1, under the experimental conditions, the hybrids prepared from the dasatinib core (fragment) showed significant efficacies against native K-562 cells. Particularly, 3d, potently inhibited the growth of native K-562 cells with a Gl₅₀ values of 30 nM. Consistent with the cellular inhibition potency, it has effectively inhibited the activity of native BCR-ABL kinase (Table 2). However, similar to dasatinib, these hybrids were also ineffective against T315I mutation; they did not inhibit the activity of the BCR-ABL^{T315I} kinase and growth of corresponding K-562 cell lines.

TABLE 1
Cellular activity of the hit finder compounds.

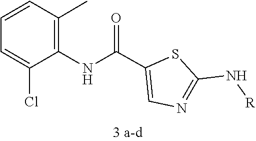

| | | GI50 Values | | |
|---|---|---|---|---|
| | | (uM ± SD) | | Cardio- |
| Com- | | | K-562- | toxicityª |
| pound | R | K-562 | T315I | (μM) |
| 3a | 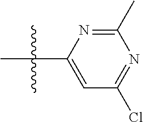 [SMILES: *c1cc(Cl)nc(C)n1] | 0.05 ± 0.04 | ND | >10 |
| 3b | 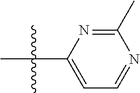 [SMILES: *c1ccnc(C)n1] | ND | ND | >10 |
| 3c | 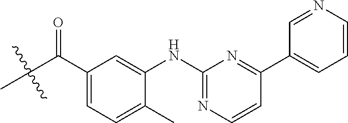 [SMILES: *C(=O)c1ccc(C)c(Nc2nccc(-c3cccnc3)n2)c1] | 0.24 ± 0.09 | 5.3 ± 1.1 μM | >10 |
| 3d | 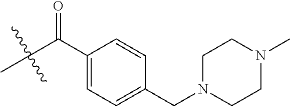 [SMILES: *C(=O)c1ccc(CN2CCN(C)CC2)cc1] | 0.03 ± 0.01 | ND | >10 |

ªOverall maximum toxic dose,
ND—No inhibition detected up to 10 μM concentration.

TABLE 2
Kinase inhibition for the selected hit finder compounds.

Kinase inhibition
(IC₅₀, nM ± SD)

| | BCR-ABL- | BCR-ABL- |
|---|---|---|
| Compound | Wt | T315I |
| 3 c | 5.02 | >10,000 |
| 3 d | 0.5 | >10,000 |
| 10 b | 3.98 | 547 |
| 10 c | 1000 | >10,000 |
| 15 | 150 | 361 |
| 20 | 58.5 | 121 |
| 21 b | 9.09 | 670 |
| 24 | 9.43 | 16.1 |
| 32 | 1.61 | 391 |

TABLE 3
Cellular activity of the hit finder compounds.

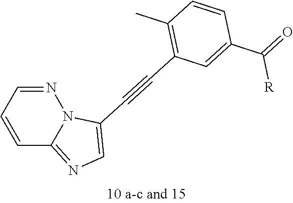

| | | GI50 Values | | |
|---|---|---|---|---|
| | | (uM ± SD) | | |
| Com- | | | K-562- | Cardiotoxicityª |
| pound | R | K-562 | T315I | (μM) |
| 10a | 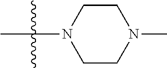 [SMILES: *N1CCN(C)CC1] | ND | ND | >10 |
| 10b | 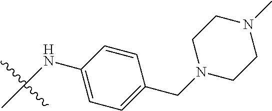 [SMILES: *Nc1ccc(CN2CCN(C)CC2)cc1] | 0.14 ± 0.09 | 3.15 ± 0.3 | >10 |
| 10c | 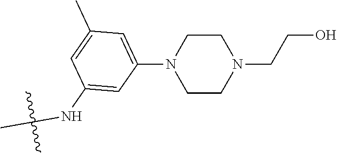 [SMILES: *Nc1cc(C)cc(N2CCN(CCO)CC2)c1] | ND | ND | 5.4 |
| 15 | 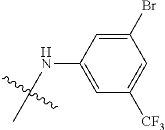 [SMILES: *Nc1cc(Br)cc(C(F)(F)F)c1] | 0.02 ± 0.006 | 0.37 ± 0.12 | >10 |

ªOverall maximum toxic dose, ND—No inhibition detected up to 10 μM concentration.

However, 3a-d appeared to be cardiac-safe hybrids (Table 1), as we did not observe voltage transients and arrhythmia up to 10 μM concentration. Furthermore, we did not observe a decrease contractility up to this dose.

TABLE 4
Cellular activity of the hit finder compounds.

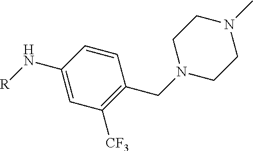

| | | GI50 Values | | |
|---|---|---|---|---|
| | | (μM ± SD) | | |
| | | | K-562- | Cardiotoxicityª |
| Compound | R | K-562 | T315I | (μM) |
| 19 | 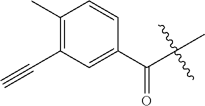 [SMILES: *C(=O)c1ccc(C)c(C#C)c1] | 7.14 ± 4.2 | 5.4 ± 2.1 | 1.13 |
| 20 | 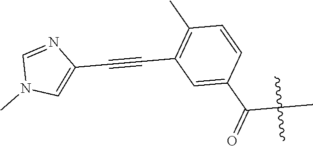 [SMILES: *C(=O)c1ccc(C)c(C#Cc2cn(C)cn2)c1] | 0.3 ± 0.15 | 1.2 ± 0.4 | 6.44 |
| 21 | 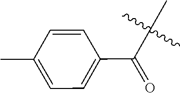 [SMILES: *C(=O)c1ccc(C)cc1] | ND | ND | 1.45 |
| 21 b | 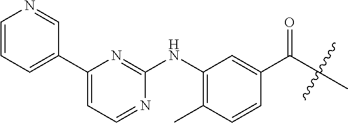 [SMILES: *C(=O)c1ccc(C)c(Nc2nccc(-c3cccnc3)n2)c1] | 0.003 ± 0.001 | 1.55 ± 0.28 | 4.34 |
| 24 | 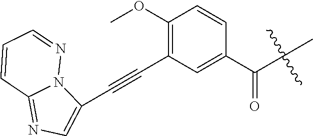 [SMILES: *C(=O)c1ccc(OC)c(C#Cc2cnc3cccnn23)c1] | 0.003 ± 0.002 | 0.13 ± 0.08 | 9.78 |
| 29 | 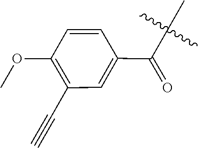 [SMILES: *C(=O)c1ccc(OC)c(C#C)c1] | ND | ND | 1.34 |
| 26 | — | ND | ND | >10 |
| 32 | — | 0.013 ± | 3.4 ± | >10 |
| | | 0.01 | 0.06 | |

ªOverall maximum toxic dose,
ND—No inhibition detected up to 10 uM concentration

Earlier findings with the dasatinib core encouraged us to explore SAR using other inhibitor's cores, which binds with Met318 in hinge region of BCR-ABL. In our next step, while we were selecting a new core, we also considered H bond interactions of the core with Met318 in BCR-ABL^{T315I} . In this context, we decided to study SAR against a ponatinib core (fragment), 3-(imidazo[1,2-b]pyridazin-3-ylethynyl)-4-methylbenzoic acid, 9. As shown in table 3, significant improvement was observed in inhibiting both native K-256 cells and K-562 cells expressing BCR-ABL^{T315I} for the hybrids made from the ponatinib core. Particularly, 15 exhibited remarkable growth inhibition against native K-562 cells and K-562 cells expressing BCR-ABL^{T315I} , with Gl₅₀ values of 20 nM and 370 nM, respectively. Highly consistent with its cellular activity, 15 also strongly inhibited the native BCR-ABL and BCR-ABL^{T315I} kinases in a biochemical kinase assay (Table-2), with IC₅₀ values of 150 nM and 361 nM, respectively. It appears that 15 can access the hydrophobic pocket of the BCR-ABL^{T315I} so that it can inhibit the growth of the BCR-ABL^{T315I} . From docking studies, we observed that 15 could interact with the key residues, such as Met318, Glu286 and Asp381 of BCR-ABLT315I via H bond interactions (Fig S-3), similar to that observed for ponatinib with BCR-ABL^{T315I} .²⁹ On the other hand 10a and 10c. the hybrids that were prepared from same ponatinib core, were found to be inactive, as they did not exhibit efficacy against K-562 cells up to 10 μM. However, except 10c, which has shown dose dependent cardiotoxicity, all of the compounds that were studied using this ponatinib core moiety have shown improved cardiac-safety over ponatinib. We speculated that 10c cardiotoxicity could have arisen from its interaction with some other targets that would potentially cause cardiotoxicity. Therefore, the fragment (R group) used to prepare 10c was avoided in the subsequent studies.

Despite being less potent than panatinb, hybrids containing moiety have exhibited significantly improved cardiac-safety over ponatinib. Therefore, we speculated that the ponatinib fragment that interacts with Met318 could be cardiac-safe and the other part of the ponatinib molecule may be liable for its cardiotoxicity. To understand further which part of the ponatinib molecule is liable for cardio-toxicity, in the next step, we decided to study SAR around the other core of ponatinib. Several diverse hybrids were prepared using 4-((4-methylpiperazin-1-yl)methyl)-3-(trifluoromethyl)aniline 17 as a core. Consistent with our hypothesis, the majority of hybrids (Table 4, 19-20, 21a-b, 24 and 29) have exhibited cardiotoxicities within the measured concentrations. Furthermore, some of the hybrids were found to be inactive against K-562 cell lines, but they have exhibited significantly higher cardiotoxicities than the hybrids that have exhibited activities in this series. For example, hybrids 21a and 29 were ineffective against K-562 cells lines up to 10 μM, but they were found to be highly cardio-toxic at a dose of 1.45 and 1.34 μM, respectively. Moreover, 21b, which is a hybrid molecule of imatinib and ponatinib had significantly instigated cardiotoxicity at 4.34 μM. These finding are clearly suggesting that the cardiotoxicity arises from fragment of 17. Because, imatinib did not exhibit cardiotoxicity up to 10 μM (table 5), whereas notable cardiotoxicity was observed for 21b at a much lower concentration than the imatinib safe dose concentration.

Despite their cardiotoxicities, the hybrids that were generated from 17, such as 20, 21b and 24 exhibited noticeable efficacies against K-562 cells, with Gl₅₀ values of 300 nM 3 nM and 3 nM, respectively. Nevertheless, except 24, none of these hybrids has shown improved efficacies over 15, against BCR-ABL^{T315I} kinase and the corresponding K-562 cell lines. However, compound 15 exhibited more favorable cardiac-safety than 24, and so its SAR was further explored.

SAR around 15: The SAR around the lead compound 15 was explored by investigating the influence of different R₁ and R₂ groups. Our computational investigation suggested that modifications at the R₁ and R₂ position could preserve all elements of molecular recognition. The new analogues could access ATP binding sites of both the BCR-ABL and BCR-ABL^{T315I} , and therefore, they would make key H bond interactions with Met318, Glu286 and Asp381 in both native BCR-ABL and BCR-ABL^{T315I} protein (Fig S-1 top, bottom). Hence, we expected either similar or enhanced potencies for the designed hybrids compared to 15. Relative to 15, most of the hybrids demonstrated improved efficacies in enzymatic and cellular assays (Table 5).

Particularly, replacing the bromo group with imidazole or substituted imidazoles at the R₂ position has dramatically enhanced the activities for the inhibitors. For example, as shown in table 5, 33a-33d and 36a have exhibited remarkably increased potencies over 15. Notably, the hybrids 33a and 36a, have shown dramatically increased potencies in both enzymatic and cellular assays, against BCR-ABL^{T315I} , with a 6-7 fold improvement compared to 15 (table 5). It was noticed that the bulkiness on the imidazole ring significantly affects the potency for these hybrids. For example, compared to 33a, the hybrids 36a, 33b-33d, 33g-h, which contains alkyl groups or bulky aromatic groups at the C-4 position of the imidazole ring were found to be less potent. Moreover, activity was gradually decreased for the hybrids by increasing alkyl chain length at this position. This phenomenon was clearly observed for both BCR-ABL and BCR-ABL^{T315I} protein inhibition, with 33d being exempt. For example, 33a with no substitution on the imidazole showed superior activity among all of the hybrids, with an IC₅₀ value of 20.1 nM and 43.7 nM for BCR-ABL and BCR-ABL^{T315I} , respectively. Whereas, 36a, with a methyl group at C-4 of the imidazole ring, was found to be slightly less potent than 33a (IC₅₀ values of 26.3 nM and 51.4 nM for BCR-ABL and BCR-ABL^{T315I} , respectively). Moreover, while the length of the alkyl chain has gradually increased in 33b and 33c by incorporating ethyl and isopropyl groups, respectively, their potencies were decreased stepwise. Finally, 33c with an isopropyl group was found to be the least potent among all n-alkyl substituted analogues (IC₅₀ values of 119 nM and 255 nM for BCR-ABL and BCR-ABL^{T315I} , respectively). Surprisingly, 33d, with a cyclopropyl substitution showed slightly better activity (IC₅₀s 88.4 nM for BCR-ABL and 164 nM for BCR-ABL^{T315I}) than the isopropyl analogue 33c.

Overall, the BCR-ABLT315I kinase activity for these hybrids was reduced by 2-3-fold than the native BCR-ABL kinase activity, similar to that observed for ponatinib.⁴¹ A slight outward displacement of the flag-methyl group containing phenyl ring of the hybrids from the hydrophobic pocket of BCR-ABL^{T315I} would account for the reduction in potency against BCR-ABL^{T315I} . Such outward displacement was observed for ponatinib in complex with BCR-ABL^{T315I} so that it had shown reduced potencies against BCR-ABL^{T315I} kinase and corresponding cell lines.⁴¹

Next, the potency impact of the bulkiness on imidazole moiety was further explored using 1H-benzo[d]imidazole (33g) and 4-phenyl-1H-imidazole (33h) moieties. As expected, both hybrids 33g and 33h showed markedly reduced kinase and cellular activities than 33a. Compared to 33a, these compounds have exhibited a decrease in potency of 7-16-fold and 4-9-fold in BCR-ABL^{T315I} enzymatic and cellular assays, respectively.

In further optimization, we sought to use the 1-methylpiperazine moiety, which is a widely used solubilizing group. We thought that its incorporation would improve cell permeability and help reduce lipophilicity. However, hybrid 33f did not show improved efficacies over 33a. Despite similar efficacy between 33a and 33f against native BCR-ABL kinase, relative to 33a, 33f demonstrated 2-fold decreased activity against BCR-ABL^{T315I} kinase. Cellular inhibition efficacies for 33f was found to be consistent with biochemical assay results. Another hybrid 33e, with 3-methyl-1H-pyrrole, was also unable to compete with 33a. Relative to 33a, 33e exhibited remarkably reduced BCR-ABL^{T315I} kinase and cellular potencies of 14-fold and 6-fold, respectively, which suggests that the 2^{nd} nitrogen in the five member ring is essential in order to improve the efficacies of the hybrids.

Next, the potency impact of the Flag-methyl⁴⁷ group (R₁) was briefly investigated to evaluate its impact on inhibitory activities. The hybrids 33a, 33d and 36a were selected for the study, and the results were summarized in table 5. When the methyl group in 33a was replaced with H, the resulting inhibitor 40a displayed similar efficacies that 33a showed against native BCR-ABL kinase but the activity against BCR-ABL^{T315I} and the corresponding cell lines were dramatically decreased. Whereas, the hybrids 40c and 36b, which were derived from 33d and 36a, respectively, maintained similar efficacies that of the corresponding methyl group containing analogues, against both native BCR-ABL and BCR-ABL^{T315I} kinases. However, their cellular potencies decreased by 2-10-fold. We observed that large hydrophobic groups at the R₁ position were detrimental to the activities on both kinase and cellular levels. For instance, relative to 33d and 36a, the methoxy analogues 40b and 36c demonstrated 8-16-fold and 35-100 fold potency loss against BCR-ABL^{T315I} kinase and the corresponding K-562 cell lines, respectively. In line with previous findings,^{43, 47-48} our results also clearly demonstrated the importance of the flag-methyl group's role in selective inhibition of BCR-ABL. Furthermore, similar to that observed for ponatinib binding with BCR-ABL,⁴¹ the flag-methyl in hybrids could favor desirable binding orientation with BCR-ABL. Therefore, replacing the flag-methyl with either H or a large hydrophobic group could result in loss of selectivity,⁴⁹ thus the corresponding hybrids were found to be less potent than their methyl group containing analogues.

### Hybrids Decreased Adverse Effects and Cardiotoxicity:

The TKIs used in CML treatment primarily target BCR-ABL kinase activity. However, most of them have shown distinctive off-target activities,^{29, 50} which result in adverse effects.³⁴ Cardiovascular complications are particularly restricting the use of the most potent TKIs.^{33, 51-52} For example, ponatinib, the only drug that targets BCR-ABL^{T315I} mutation has been restricted due to cardiovascular adverse events.^{33, 51} In fact, ponatinib was reported to be the most cardiotoxic TKI among the FDA approved TKIs.³³ Ponatinib cardio-toxic events were observed at a low dose of 470 nM in vitro (Tabel 5). Furthermore, ponatinib inhibited the growth of healthy HEK cells at 1.1 μM as demonstration of its toxicity and off-target effects. By contrast, most of the hybrids, which have shown excellent efficacies against both BCR-ABL^{T315I} kinase and corresponding K-562 cells lines were found to be safer compared to ponatinib. They did not inhibit the growth of HEK cells even at 10 μM.

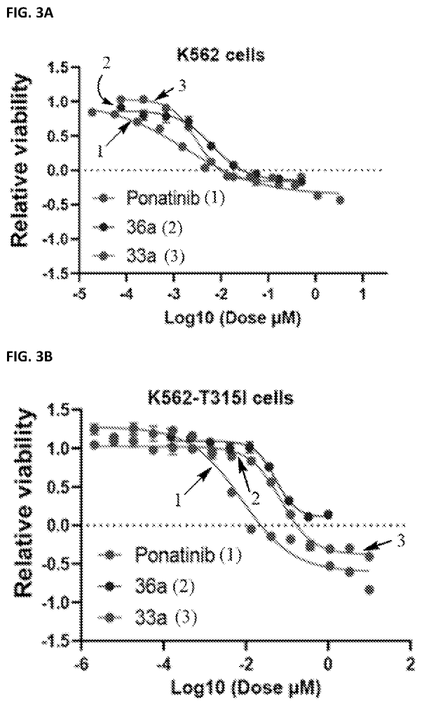

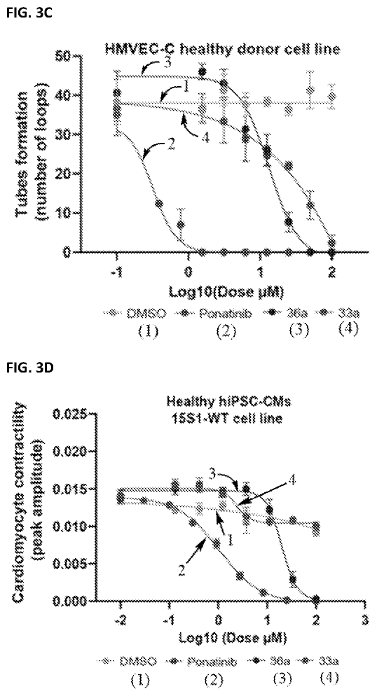

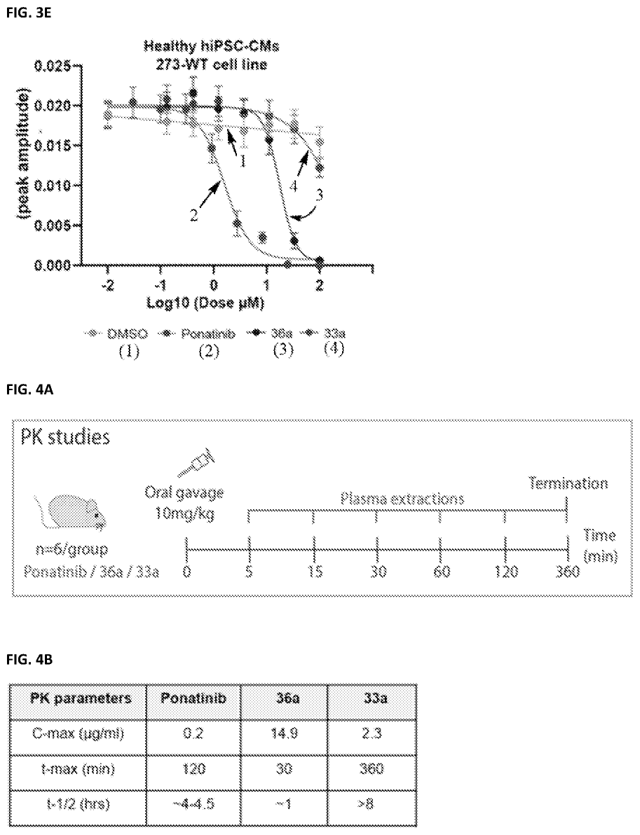

In addition, some of the hybrids found to be cardiac-safe, which have not shown adverse cardio-toxic events up to 10 μM, potently inhibited BCR-ABL^{T315I} kinase and the corresponding K-562 cell lines in the nano molar range. Particularly, the highly potent hybrids 33a and 36a have shown superior cardio-safety; we did not observe voltage transients, arrhythmia and decreasing in contractility up to 25 μM (FIG. 3). The compounds were assessed for cardiotoxic activity by measuring contractility of human cardiomyocytes derived from human induced pluripotent stem cells (hiPSC-CMs) (FIGS. 3C-3E). Note that the new compounds showed substantially diminished potencies for inhibiting cardiomyocyte contractility. Furthermore, we observed that the hybrids cardiotoxicity was also dependent on substituents at C-4 of the imidazole ring. The hybrids with more bulky groups at this position were found to be highly cardiotoxic than the unsubstituted or small substitutions. For example, hybrids 33a, 36a and 33b, with H—, methyl- and ethyl- groups, respectively, have shown cardiac-safety up to 10 μM, whereas, 33c with an isopropyl group demonstrated approximately 3-fold increased cardiotoxicity (Table 5). It exhibited cardiotoxic effects at as low as 3.5 μM, suggesting that even a small modification on the imidazole ring could cause a drastic change in the cardiac-safety. The cardiotoxicities caused by the bulkiness on the imidazole ring was clearly observed for the hybrids 33g and 33h. Particularly, 33h with the more bulky phenyl group on the imidazole moiety was found to be the most cardiotoxic hybrid among the hybrids that were studied under lead optimization. Notably, replacing the flag-methyl group with H or a methoxy group also resulted in cardiotoxicity. These findings are clearly suggest that a small change in the inhibitor structure could alter its preference in interacting with targets and off-targets,⁴⁹ and such interactions may cause adverse effects. Therefore, cardiotoxicities observed for some of the hybrids might result from their strong interactions with off-targets rather than BCR-ABL.

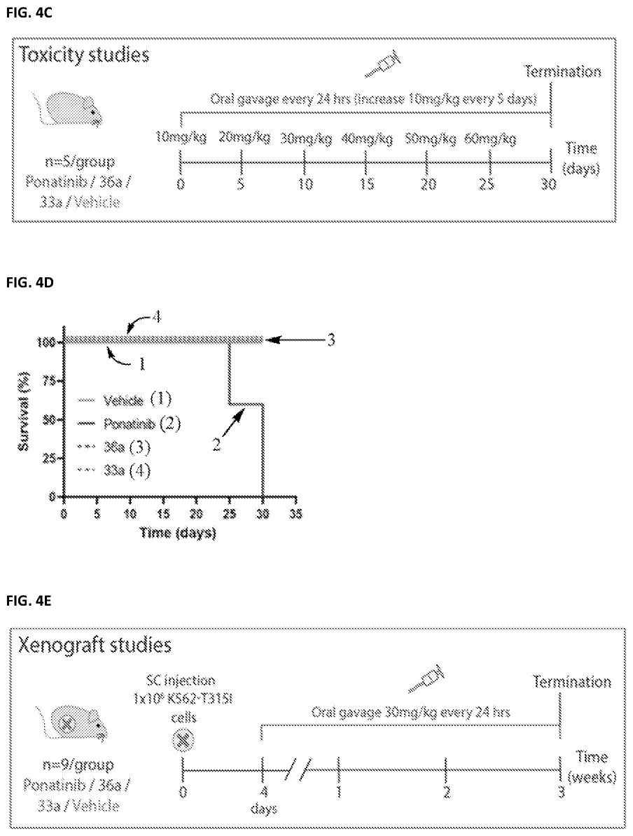

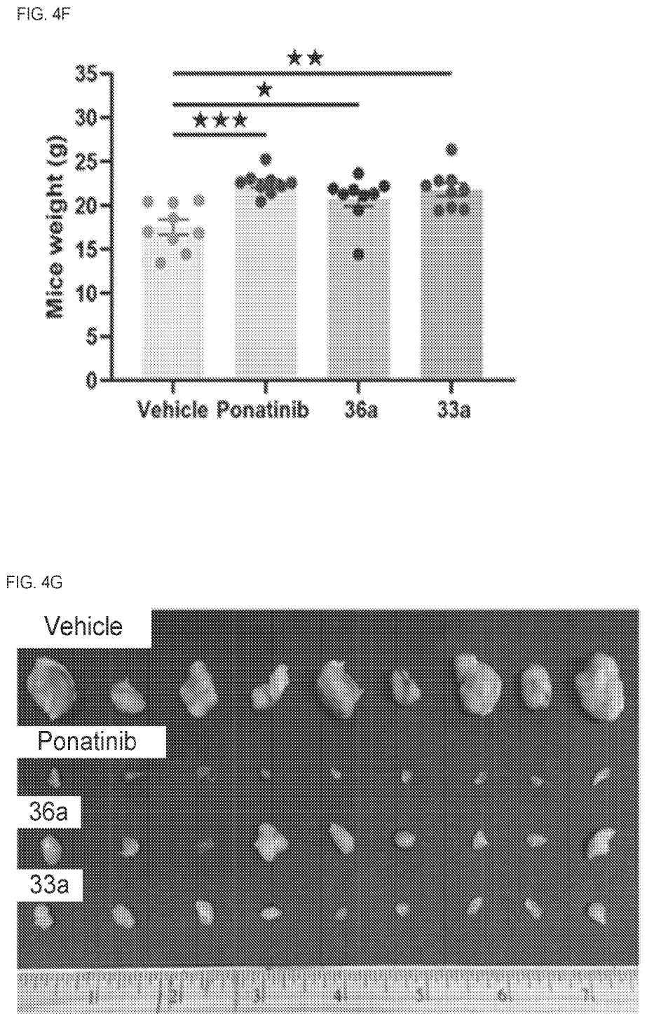

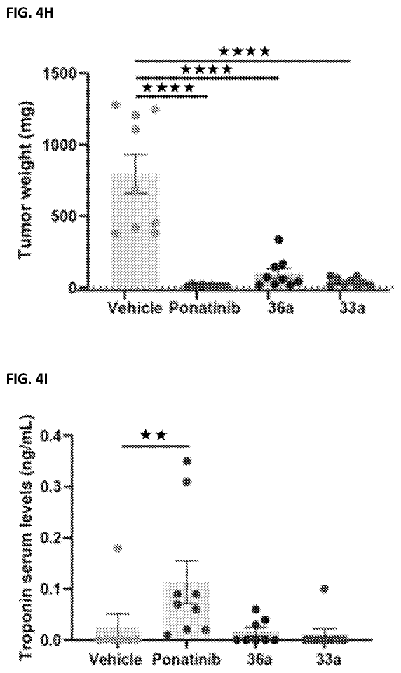

FIG. 3 represents in vitro functional evaluation of Ponatinib and compounds 33a and 36a. FIGS. 3A, 3B) Representative dose responses of Ponatinib, 33a and 36a to assess relative cell viability in CML tumor cell line K562 cells (FIG. 3A) and in the same line carrying the T315I ‘gatekeeper’ mutation (K562-T315I) (FIG. 3B). Note that 33a and 36a, like Ponatinib, are potent inhibitors of T315I mutant tumor cell growth. FIG. 3C) Representative dose responses of Ponatinib, 33a, 36a and control for angiogenesis by measuring the number of loops that form in Human Microvascular Endothelial cell cultures. Ponatinib has a potent inhibitory effect against angiogenesis but 33a and 36a show markedly diminished anti-angiogenesis potency. FIGS. 3D, dE) Representative dose responses of Ponatinib, 33a, 36a and vehicle control (DMSO) on contractility (peak contraction amplitude) of cardiomyocytes (hiPSC-CMs) generated from two different healthy donors. Each data point represents the average for 3 differentiation batches assessed each in quadruplicate (n=12). Note that Ponatinib potently suppresses cardiomyocyte contractility, whereas, 33a and 36a have substantially decreased inhibitory potencies. Considering the overall performance, including in vitro kinase and cellular potencies as well as cardiac-safety, the inhibitors 33a and 36a were selected for evaluating their pharmacokinetic profiles and antitumor activities in vivo (FIG. 4). Our results suggest that these compounds have shown comparable efficacies similar to ponatinib in mouse models of CML driven by the T315I mutation. The new compounds were assessed for PK and toxicity (FIGS. 4A-4D). The new compounds were found to have desirable PK and toxicity properties. When evaluated for anti-tumor effects in vivo, mice were implated with human K-562 CML T315I mutant tumors which were allowed to develop for 4 days (FIG. 4E). At that time, compounds were administered at 30 mg/kg daily by oral gavage. Note that the new compounds and ponatinib effectively decreased tumor burden, reflected by weight gain in the mice and decreased tumor size (FIGS. 4F-4H). Serum cardiac troponin levels were assessed. Troponin levels are an indication of cardiac damage. Note that ponatinib, but not the new compounds, increased troponin levels.

In summary, we have successfully designed and synthesized a series of hybrid molecules as more selective BCR-ABL inhibitors. The hybrids maintain significant inhibition activities against K-562 human CML cells including the most intractable gatekeeper T315I mutant associated with disease progression in CML. The most potent compounds 33a and 36a strongly inhibited the kinase activities of both native BCR-ABL and BCR-ABLT315I with pharmacokinetics and achieved durable tumor regression in the K-562 xenograft model in mice with oral administration.

TABLE 5
Cellular activity of the hit finder compounds.

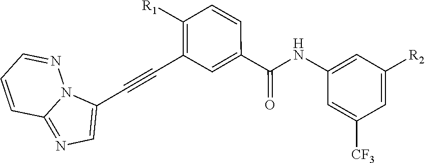

| | | | Cell | | | | | |
|---|---|---|---|---|---|---|---|---|
| | | | Inhibition | | | | HEK | |
| | | | (IC₅₀ | | Kinase Inhibition | | Cell | |
| | | | nM ± SD) | | (IC₅₀ nM ± SD) | | Inhib. | Cardio- |
| | | | | K-562- | BCR- | BCR- | (IC₅₀ | toxicity |
| Comp. | R₁ | R₂ | K562 | T315I | ABL^{WT} | ABLT315I | nM ± SD) | (μM) |
| 33a | 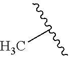 | 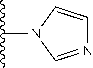 [SMILES: *n1ccnc1] | 3 ± 3 | 48 ± 30 | 20.1 | 43.7 | >10 | >10 |
| 33b | 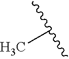 | 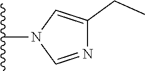 [SMILES: *n1cnc(CC)c1] | 8 ± 5 | 63 ± 58 | 66.1 | 149 | 9.27 ± 1.09 | >10 |
| 33c | 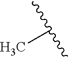 | 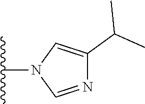 [SMILES: *n1cnc(C(C)C)c1] | 9 ± 8 | 85 ± 61 | 119 | 255 | 8.03 | 3.56 |
| 33d | 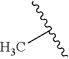 | 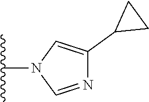 [SMILES: *n1cnc(C2CC2)c1] | 7 ± 2 | 28 ± 7 | 88.4 | 164 | 7.55 ± 1.72 | >10 (>25) |
| 33e | 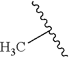 | 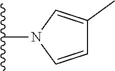 [SMILES: *n1ccc(C)c1] | 30 ± 15 | 299 ± 216 | 182 | 626 | >10 | 2.32 |
| 33f | 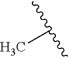 | 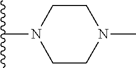 [SMILES: *N1CCN(C)CC1] | 5 ± 0.5 | 153 ± 107 | 18 | 110 | 4.02 ± 0.63 | >10 (>25) |
| 33g | 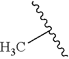 | 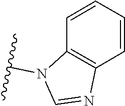 [SMILES: *n1cnc2ccccc21] | 15 ± 2 | 419 ± 94 | 144 | 364 | >10 | 4 |
| 33h | 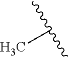 | 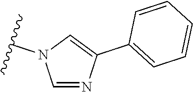 [SMILES: *n1cnc(-c2ccccc2)c1] | 21 ± 5 | 189 ± 62 | 600 | 717 | >10 | 1.2 |
| 36a | 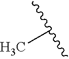 | 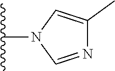 [SMILES: *n1cnc(C)c1] | 6 ± 6 | 72 ± 46 | 26.3 | 51.4 | 2.92 ± 0.78 | >10 |
| 36b | 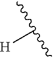 [SMILES: *[H]] | 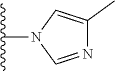 [SMILES: *n1cnc(C)c1] | 14 ± 4 | 217 ± 117 | 29.3 | 56.8 | >10 | 8.25 |
| 36c | 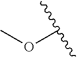 [SMILES: *OC] | 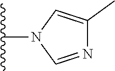 [SMILES: *n1cnc(C)c1] | 42 ± 2 | 2700 ± 600 | 92.5 | 445 | >10 | 4.65 |
| 40a | 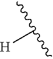 [SMILES: *[H]] | 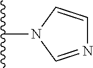 [SMILES: *n1ccnc1] | 37 ± 6 | 508 ± 200 | 25.8 | 111 | >10 | 6.9 |
| 40b | 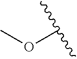 [SMILES: *OC] | 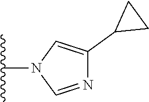 [SMILES: *n1cnc(C2CC2)c1] | 56 ± 19 | 2800 ± 970 | 116 | 710 | >10 | 2.49 |
| 40c | 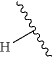 [SMILES: *[H]] | 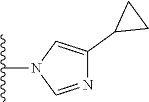 [SMILES: *n1cnc(C2CC2)c1] | 24 ± 19 | 258 ± 74 | 94 | 135 | 5.5 | 5.26 |
| Ponatinib | — | — | 0.5 ± | 4 ± 4 | 2.22 | 5.08 | 1.12 ± | 0.47 |
| | | | 0.3 | | | | 0.72 | |
| Imatinib | — | — | 563 ± | >10 | — | — | >10 | >10 |
| | | | 135 | | | | | |
| Dasatinib | — | — | 0.4 ± | >10 | — | — | | >10 |
| | | | 0.1 | | | | | |
| Nilotinib | — | — | 26 ± | >10 | — | — | 8.3 | 1.12 |
| | | | 15 | | | | | |
| Asciminib | — | — | — | — | — | — | >10 | 4.81 |

ªOverall maximum toxic dose,
ND—No inhibition detected up to 10 μM concentration.

Therefore, they could serve as promising lead compounds for further development of a new class of BCR-ABL inhibitors overcoming the T315I mutation and cardiotoxicity.

### Experimental Section

### Chemical Synthesis

General methods: All the reagents and solvents were obtained at the highest commercial quality from sources such as Sigma-Aldrich, Fisher Scientific, TCI International, Acros organics, Alfa-Aesar, Matrix Scientific, Chem-Implex and Enamine and were used without further purification. Unless otherwise mentioned, all the reactions were carried out under a nitrogen atmosphere with dry solvents. The reactions were monitored by TLC using pre-coted silica gel plates (Merck, silica gel 60 F₂₅₄). Flash chromatography was carried out using a CombiFlash Rf+Lumen chromatography system (Teledyne ISCO, Lincon, NE, USA). ¹H (400 MHZ) and ¹³C (101 MHz) NMR spectra were recorded either on an Agilent 400-MR NMR or on a Bruker Avance 400 MHz spectrometer, using appropriate deuterated solvents, as needed. Chemical shifts (δ) were reported in parts per million (ppm) upfield from tetramethylsilane (TMS) as an internal standard. Coupling constants (J) were reported in hertz (Hz), and s, br.s, d, t and m are designated as singlet, broad singlet, doublet, triplet and multiplet, respectively. LC-MS spectra were recorded on an Agilent 6490 iFunnel Triple Quadrupole Mass Spectrometer from Agilent Technologies Inc. (Santa Clara, CA, USA). An Agilent EclipsePlusC₁₈ reverse phase column, 1.8 μm, 2.1×50 mm was used with solvent A (0.1% formic acid in acetonitrile) and solvent B (0.1% formic acid in water) for LC-MS analysis. The ratio of solvent A and solvent B was 1:9 at the beginning and gradually changed to 9:1 at the end. The purity of all the final compounds was >95% as indicated by LC-MS.

N-(2-chloro-6-methylphenyl)-2-((2-methylpyrimidin-4-yl)amino) thiazole-5-carboxamide (3b). Compound 3a was prepared based on a literature procedure.42 Sodium hydride (60% in mineral oil, 0.186 g, 4.67 mmol) was added to a stirred solution of 2-amino-N-(2-chloro-6-methylphenyl)thiazole-5-carboxamide 1 (0.5 g, 1.87 mmol) and 4-chloro-2-methylpyrimidine 2b (0.28 g, 2.24 mmol) in DMF (20 mL). The solution was heated at 100° C. overnight, cooled to room temperature (rt), and quenched by adding glacial acetic acid and water. The crude product extracted into DCM (2×50 mL). The organic layers were combined, washed with water, followed by saturated NaCl solution (25 mL). The organic phase was dried over Na₂SO₄, filtered, and then evaporated to dryness using a rotatory evaporator. The crude product was purified on a silica gel column with a 0-10% gradient of methanol in DCM to furnish the desired product as pale yellow solid (0.07 g, 10% yield). ¹H NMR (400 MHZ, DMSO-d₆) δ8.37 (d, J=5.5 Hz, 1H), 8.12 (s, 1H), 7.58-7.48 (m, 1H), 7.47-7.37 (m, 2H), 6.90 (dd, J=5.5, 0.7 Hz, 1H), 2.49 (s, 3H), 2.14 (s, 3H). ¹³C NMR (101 MHZ, DMSO-d₆) δ171.60, 167.08, 164.66, 159.64, 157.56, 156.38, 138.75, 132.04, 131.71, 131.34, 130.68, 128.57, 114.29, 98.62, 25.91, 17.76. LC-MS (ESI-QQQ): m/z 360.1 ([C₁₆H₁₄ClN₅OS+H]⁺ calcd. 360.06). Purity 99% (RT 3.287 min).

### General Procedure for the Synthesis of 3c-d.

The following procedure is for N-(2-chloro-6-methylphenyl)-2-(4-methyl-3-((4-(pyridin-3-yl)pyrimidin-2-yl)amino)benzamido)thiazole-5-carboxamide (3c). Under a nitrogen atmosphere, 2-amino-N-(2-chloro-6-methylphenyl)thiazole-5-carboxamide 1 (0.5 g, 1.87 mmol) and 4-methyl-3-((4-(pyridin-3-yl)pyrimidin-2-yl)amino)benzoic acid 2c (0.57 g, 1.87 mmol) were added to dry THF (100 mL) at room temperature and stirred for 10 min, which resulted in a clear solution. EDC.HCl (0.54 g, 2.80 mmol), HOBt (0.38 g, 2.80 mmol) and DIPEA (0.65 mL, 3.74 mmol) were added and then heated at 40° C.for 48 h. The progress of the reaction was monitored by TLC. Water (25 mL) was added, followed by EtOAc (25 mL). The organic phase was separated and the aqueous phase was extracted with EtOAc (2×50 mL). The combined organic phase was washed with water (25 mL) followed by brine solution (25 mL). The organic phase was dried over Na₂SO₄, filtered and evaporated to dryness to afford crude product that was purified on a silica gel column with a 0-10% gradient of methanol in DCM as an eluent to obtain the desired compound as an off-white solid (0.08 g, 8% yield). ¹H NMR (400 MHZ, DMSO-d₆) δ12.91 (s, 1H), 9.75 (s, 1H), 9.29 (s, 1H), 9.06 (s, 1H), 8.70 (d, J=4.7 Hz, 1H), 8.53 (dd, J=16.2, 6.6 Hz, 2H), 8.45 (s, 1H), 8.27 (s, 1H), 7.89 (d, J=7.9 Hz, 1H), 7.56 (t, J=6.5 Hz, 1H), 7.48 (d, J=5.2 Hz, 1H), 7.38 (dd, J=13.9, 7.8 Hz, 2H), 7.33-7.21 (m, 2H), 2.34 (s, 3H), 2.26 (s, 3H). ¹³C NMR (101 MHz, DMSO-d₆) δ162.00, 161.58, 159.96, 151.85, 148.56, 139.29, 138.19, 134.87, 134.33, 132.92, 132.63, 130.44, 129.38, 128.41, 127.38, 125.38, 124.60, 124.36, 108.14, 40.58, 40.37, 40.16, 39.95, 39.74, 39.53, 39.33, 18.83, 18.68. LC-MS (ESI-QQQ): m/z 556.20 ([C₂₈H₂₂ClN₇O₂S+H]⁺ calcd. 556.12). Purity 96.3% (RT 4.853 min).

N-(2-chloro-6-methylphenyl)-2-(4-((4-methylpiperazin-1-yl)methyl)benzamido)thiazole-5-carboxamide (3d). The title compound was synthesized from 2-amino-N-(2-chloro-6-methylphenyl)thiazole-5-carboxamide 1 (0.62 g, 2.34 mmol) and 4-((4-methylpiperazin-1-yl)methyl)benzoic acid 2d (0.5 g, 2.13 mmol), as described for the synthesis of 3c. The crude product was purified on a silica gel column using a 0-10% gradient of methanol in DCM as an eluent to yield the desired compound as an off-white solid (0.2 g, 19% yield). ¹H NMR (400 MHZ, DMSO-d₆) δ10.09 (s, 1H), 8.39 (s, 1H), 8.13-8.03 (m, 2H), 7.47 (d, J=8.2 Hz, 2H), 7.41 (dd, J=7.5, 2.0 Hz, 1H), 7.35-7.20 (m, 3H), 3.55 (s, 2H), 2.41 (bs, 8H), 2.25 (s, 3H), 2.21 (s, 3H). ¹³C NMR (101 MHZ, DMSO-d₆) δ165.82, 162.40, 159.97, 144.14, 141.02, 139.20, 133.75, 132.81, 130.82, 129.53, 129.27, 128.78, 127.50, 127.30, 123.75, 118.99, 110.92, 61.86, 54.93, 52.71, 45.83, 18.74. LC-MS (ESI-QQQ): m/z 484.10 ([C₂₄H₂₆ClN₅O₂S+H]⁺ calcd. 484.15). Purity 97.9% (RT 3.520 min).

3-ethynylimidazo[1,2-b]pyridazine (5). Compound 5 was prepared according to the previously reported method,⁴³ with several modifications. To a solution of 3-bromoimidazo[1,2-b]pyridazine 4 (10.0 g, 50.5 mmol) in acetonitrile was added Cul (0.5 g, 2.63 mmol), Pd(PPh₃)₂Cl₂ (1.8 g 2.63 mmol) and TEA (21.0 mL, 150.6 mmol). The solution was purged with a nitrogen flow for 10 min and then ethynyltrimethylsilane (21.0 mL, 151.8 mmol) was added. The mixture was heated to reflux overnight. After cooling to rt, the reaction mixture was filtered to remove undissolved solid. The solid was washed with copious amounts of acetonitrile. The filtrate was evaporated to dryness then taken into methanol (300 mL). To this mixture, K₂CO₃ (14.3 g, 103.5 mmol) was added at room temperature and then allowed to stir for 4 h. The progress of the reaction was monitored by TLC. The reaction mixture was filtered in order to remove excess K₂CO₃. The solid was washed with a minimal amounts of methanol. The filtrate was concentrated to dryness and dissolved in excess EtOAc, and then washed with water followed by brine solution. The organic phase was dried over Na₂SO₄, filtered and evaporated to dryness to afford crude product, which was purified on a silica gel column using a 0-50% gradient of EtOAc in hexane to obtain the desired product as a pale-brown solid (5.0 g, 69%). ¹H NMR (400 MHZ, CDCl₃) δ8.47 (dd, J=4.4, 1.7 Hz, 1H), 8.03-7.96 (m, 2H), 7.12 (dd, J=9.1, 4.5 Hz, 1H), 3.80 (s, 1H). ¹³C NMR (101 MHZ, CDCl₃) δ143.92, 138.97, 132.13, 132.03, 128.53, 128.41, 126.04, 117.86, 87.25, 70.61. LC-MS (ESI-QQQ): m/z 144.10 ([C₈H₅N₃+H]⁺ calcd. 144.05). Purity 99% (RT 2.680 min).

Methyl 3-(imidazo[1,2-b]pyridazin-3-ylethynyl)-4-methylbenzoate (8). Compound 8 was prepared according to the literature procedure,⁵³ with few modifications. Methyl 3-iodo-4-methylbenzoate 6 (1.85 g, 6.71 mmol) was added to a stirred solution of 3-ethynylimidazo[1,2-b]pyridazine 5 (0.8 g, 5.59 mmol) in DMF (10 mL). The mixture underwent 3 cycles of vacuum/filling with nitrogen and then Cul (0.21 g, 1.11 mmol), Pd(PPh₃)₄ (0.64 g, 0.55 mmol) and diisopropylethylamine (1.94 mL, 11.17 mmol) were added. The reaction mixture was stirred at 80° C. for 2 h before it was cooled to rt. Water (25 mL) was added and the product extracted into EtOAc (3×25 mL). The organic layers were combined, washed with water (20 mL) followed by brine solution (20 mL). The organic phase was dried over Na₂SO₄, filtered and then evaporated to dryness to afford a gummy solid, which was then triturated with minimal acetonitrile to yield a solid. The solid was collected by filtration, was washed with a minimal amount of acetonitrile and dried under vacuum for 2 h to furnish the desired compound as an off-white solid (0.82 g, 50% yield). ¹H NMR (400 MHZ, CDCl₃) δ8.57 (d, J=4.2 Hz, 1H), 8.27 (d, J=1.8 Hz, 1H), 8.16 (s, 1H), 7.97-7.87 (m, 1H), 7.39-7.30 (m, 1H), 7.26 (s, 1H), 7.22 (s, 1H), 3.93 (d, J=0.5 Hz, 3H), 2.63 (d, J=0.7 Hz, 3H). ¹³C NMR (101 MHZ, CDCl₃) δ166.37, 145.55, 144.26, 133.19, 129.90, 129.76, 127.99, 125.64, 122.47, 118.15, 94.49, 52.17, 21.06. LC-MS (ESI-QQQ): m/z 292.00 ([C₁₇H₁₃N₃O₂+H]⁺ calcd. 292.10). Purity 99% (RT 5.027 min).

3-(imidazo[1,2-b]pyridazin-3-ylethynyl)-4-methylbenzoic acid (9). Compound 9 was prepared based on a literature procedure,⁵³ with few modifications. Methyl 3-(imidazo[1,2-b]pyridazin-3-ylethynyl)-4-methylbenzoate 8 (0.81 g, 2.78 mmol) was taken into a 1:1 mixture of MeOH and THF (120 mL). To this mixture, a freshly prepared 1.0 M LiOH solution in water (15.0 mL) was added and stirred at rt for 24 h. The pH was adjusted to 2 before the volume was reduced to 15% on a rotatory evaporator. The off-white solid that had appeared was collected by filtration, washed with copious amounts of ether and dried under vacuum for 4 h to give the title compound (0.7 g, 91% yield). ¹H NMR (400 MHZ, DMSO-d₆) δ13.08 (s, 1H), 8.70 (dd, J=4.4, 1.6 Hz, 1H), 8.28-8.15 (m, 2H), 8.03 (d, J=1.8 Hz, 1H), 7.87 (dd, J=7.9, 1.9 Hz, 1H), 7.48 (d, J=8.0 Hz, 1H), 7.37 (dd, J=9.2, 4.4 Hz, 1H), 2.57 (s, 3H). ¹³C NMR (101 MHZ, DMSO-d₆) δ166.92, 145.52, 144.85, 140.11, 138.74, 132.38, 130.68, 130.11, 129.33, 126.53, 122.48, 119.51, 112.14, 96.61, 81.55, 20.94. LC-MS (ESI-QQQ): m/z 277.9 ([C₁₆H₁₁N₃O₂+H]⁺ calcd. 278.09). Purity 99% (RT 4.16 min).

Compounds 11a-c were prepared from compound 9 and the corresponding reactant 10 using a similar method that was described for the synthesis of 3d.

(3-(imidazo[1,2-b]pyridazin-3-ylethynyl)-4-methylphenyl)(4-methylpiperazin-1-yl)methanone (11 a). Compound 11a was prepared using 3-(imidazo[1,2-b]pyridazin-3-ylethynyl)-4-methylbenzoic acid 9 (0.1 g, 0.36 mmol) and 1-methylpiperazine (0.04 g, 0.54 mmol) as shown in scheme 2. Desired product was obtained as an off-white solid (0.05 g, 39% yield). ¹H NMR (400 MHZ, CDCl₃) δ8.46 (dd, J=4.4, 1.6 Hz, 1H), 8.03 (s, 1H), 7.99 (dd, J=9.2, 1.6 Hz, 1H), 7.62 (d, J=1.3 Hz, 1H), 7.30 (d, J=1.5 Hz, 2H), 7.12 (dd, J=9.2, 4.4 Hz, 1H), 3.66 (m, 8H), 2.59 (s, 3H), 2.34 (s, 3H). ¹³C NMR (101 MHZ, CDCl₃) δ169.44, 143.84, 142.07, 139.69, 138.30, 133.28, 130.40, 129.77, 127.50, 125.90, 122.66, 117.65, 96.84, 80.55, 45.89, 20.78. LC-MS (ESI-QQQ): m/z 360.3 ([C₂₁H₂₁N₅O+H]⁺ calcd. 360.17). Purity 99% (RT 3.06 min).

3-(imidazo[1,2-b]pyridazin-3-ylethynyl)-4-methyl-N-(4-((4-methylpiperazin-1-yl)methyl)phenyl)benzamide (11 b). Compound 11 b was prepared using 3-(imidazo[1,2-b]pyridazin-3-ylethynyl)-4-methylbenzoic acid 9 (0.1 g, 0.36 mmol) and 4-((4-methylpiperazin-1-yl)methyl)aniline (0.07 g, 0.36 mmol) as shown in scheme 2. Desired product was obtained as an off-white solid (0.02 g, 12% yield). ¹H NMR (400 MHZ, DMSO-d₆) δ10.28 (s, 1H), 8.71 (dd, J=4.4, 1.6 Hz, 1H), 8.31-8.11 (m, 3H), 7.91 (dd, J=8.0, 2.0 Hz, 1H), 7.76-7.63 (m, 2H), 7.52 (dd, J=8.0, 0.8 Hz, 1H), 7.38 (dd, J=9.2, 4.5 Hz, 1H), 7.29-7.18 (m, 2H), 3.40 (s, 2H), 2.59 (s, 3H), 2.32 (d, J=13.3 Hz, 8H), 2.15 (s, 3H). ¹³C NMR (101 MHz, DMSO-d₆) δ145.52, 138.69, 138.31, 133.19, 130.60, 130.43, 129.54, 128.88, 126.56, 122.14, 120.66, 119.52, 62.07, 55.10, 52.82, 20.82. LC-MS (ESI-QQQ): m/z 465.0 ([C₂₈H₂₈N₆O+H]⁺ calcd. 465.2). Purity 99% (RT 3.557 min).

N-(6-(4-(2-hydroxyethyl)piperazin-1-yl)-2-methylpyrimidin-4-yl)-3-(imidazo[1,2-b]pyridazin-3-ylethynyl)-4-methylbenzamide (11c). Compound 11c was prepared from 3-(imidazo[1,2-b]pyridazin-3-ylethynyl)-4-methylbenzoic acid 9 (0.1 g, 0.36 mmol) and 2-(4-(6-amino-2-methylpyrimidin-4-yl)piperazin-1-yl)ethan-1-ol (0.09 g, 0.36 mmol) as shown in scheme 2. Desired product was obtained as an off-white solid (0.04 g, 22% yield). ¹H NMR (400 MHZ, DMSO-d₆) δ8.70 (dd, J=4.5, 1.6 Hz, 1H), 8.28-8.16 (m, 2H), 8.09-8.00 (m, 1H), 7.89 (dd, J=7.9, 1.9 Hz, 1H), 7.59-7.44 (m, 1H), 7.42-7.28 (m, 1H), 6.05 (s, 2H), 5.41 (s, 1H), 4.41 (t, J=5.8 Hz, 2H), 3.39 (t, J=4.8 Hz, 4H), 2.73 (t, J=5.8 Hz, 2H), 2.58 (s, 3H), 2.51 (t, J=5.9 Hz, 4H), 2.13 (s, 3H). ¹³C NMR (101 MHZ, DMSO-d₆) δ165.88, 165.37, 164.90, 163.17, 145.53, 145.42, 138.82, 132.13, 130.89, 129.96, 126.55, 119.56, 96.44, 81.82, 79.95, 62.80, 56.49, 53.00, 44.07, 26.09, 21.00. LC-MS (ESI-QQQ): m/z 497.40 ([C₂₇H₂₈N₈O₂+H]⁺ calcd. 497.23). Purity 99% (RT 3.230 min).

N-(3-bromo-5-(trifluoromethyl)phenyl)-3-iodo-4-methylbenzamide (14). Under a nitrogen atmosphere, 3-iodo-4-methylbenzoic acid 12 (5.0 g, 19.08 mmol) was taken in SOCl₂ (6.5 mL, 89.6 mmol) and then two drops of DMF was added at rt. The reaction mixture was stirred at reflux for 5 h before it was cooled to rt and the excess SOCl₂ was carefully removed. The crude material was co-evaporated with benzene and dried under vacuum to afford the desired acid chloride. The acid chloride was dissolved in anhydrous THF (20 mL) and then added dropwise to a stirred mixture of 3-bromo-5-(trifluoromethyl)aniline 13 (4.57 g, 19.08 mmol), diisopropylethylamine (3.97 mL, 22.8 mmol) and DMAP (0.23 g, 1.88 mmol) in THF at 0° C. Upon completion of the addition, the reaction mixture was warmed to rt and stirred overnight. The reaction was quenched with water, and the product was extracted into EtOAc (3×50 mL). The combined organic extracts were washed with brine solution (25 mL), dried over Na₂SO₄, filtered and evaporated to dryness to afford a crude material that was purified on a silica gel column using a 0-50% gradient of EtOAc in hexane as eluent to obtain the desired product as an off-white solid (7.6 g, 82% yield). ¹H NMR (400 MHZ, DMSO-d₆) δ10.61 (s, 1H), 8.49-8.29 (m, 2H), 8.19 (s, 1H), 7.91 (dd, J=7.9, 1.9 Hz, 1H), 7.67 (s, 1H), 7.50 (d, J=7.9 Hz, 1H), 2.44 (s, 3H). ¹³C NMR (101 MHZ, DMSO-d₆) δ164.63, 145.89, 141.74, 137.94, 133.53, 131.73, 131.41, 130.35, 128.34, 126.48, 124.98, 123.04, 123.00, 122.67, 115.92, 115.88, 101.63, 28.06. LC-MS (ESI-QQQ): m/z 483.90 ([C₁₅H₁₀BrF₃INO+H]⁺ calcd. 483.89). Purity 99% (RT 6.410 min).

N-(3-bromo-5-(trifluoromethyl)phenyl)-3-(imidazo[1,2-b]pyridazin-3-ylethynyl)-4-methylbenzamide (15). This was prepared using N-(3-bromo-5-(trifluoromethyl)phenyl)-3-iodo-4-methylbenzamide 14 (2.0 g, 4.13 mmol) and 3-ethynylimidazo[1,2-b]pyridazine 5 (0.62 g, 4.33 mmol) as shown in scheme 3 using a similar method that was described for the synthesis of 8. The desired product was obtained as an off-white solid (1.42 g, 69% yield). ¹H NMR (400 MHZ, DMSO-d₆) δ10.69 (s, 1H), 8.72 (dd, J=4.4, 1.5 Hz, 1H), 8.39 (t, J=1.9 Hz, 1H), 8.31-8.17 (m, 3H), 7.94 (dd, J=8.0, 2.0 Hz, 1H), 7.69-7.52 (m, 3H), 7.39 (dd, J=9.2, 4.4 Hz, 1H), 2.61 (s, 3H). ¹³C NMR (101 MHZ, DMSO-d₆) δ165.32, 145.51, 144.31, 141.81, 138.74, 132.20, 131.98, 131.88, 131.71, 131.39, 130.63, 130.61, 129.26, 129.14, 128.99, 126.56, 126.44, 122.66, 122.31, 119.54, 115.85, 96.76, 81.70, 20.87. LC-MS (ESI-QQQ): m/z 499.1 ([C₂₃H₁₄BrF₃N₄O+H]⁺ calcd. 499.03). Purity 95.8% (RT 6.040 min).

3-ethynyl-4-methylbenzoic acid (16). Methyl 3-iodo-4-methylbenzoate 6 (3.0 g, 10.86 mmol), was taken in anhydrous THF (30 mL). The solution underwent 3 cycles of vacuum/filling with nitrogen then Cul (0.17 g, 0.89 mmol), [Pd(PPh₃)₂Cl₂] (0.4 g, 0.56 mmol) and ethynyltrimethylsilane (5.0 mL, 36.14 mmol) were added. The mixture was stirred overnight at rt. EtOAc (50 mL) was added followed by 0.5 M aqueous NH₄OH solution (100.0 mL). Aqueous and organic phases were separated. The organic phase was washed with 0.5N HCl (50 mL) followed by brine solution (25 mL), dried over Na₂SO₄, filtered and evaporated to dryness to afford a brown oil that was dissolved in a freshly prepared methanolic KOH solution (13 g of KOH flakes dissolved in 50 ml of MeOH) and stirred at rt for 2 h. EtOAc (100 mL) was added and undissolved solid was removed by filtration. The solid was washed with copious amounts of methanol. The filtrate was evaporated to dryness and taken in water (50 mL). The pH was adjusted to 5 using 0.5N HCl, during which time an off-white solid was observed. The solid obtained was collected by filtration and washed with cold water followed by hexane. The solid was dried under vacuum for 4 h to obtain the desired compound as an off-white solid (1.5 g, 86%). ¹H NMR (400 MHZ, DMSO-d₆) δ13.10 (s, 1H), 7.92 (d, J=1.8 Hz, 1H), 7.84 (dd, J=8.0, 1.8 Hz, 1H), 7.42 (d, J=8.0 Hz, 1H), 4.47 (d, J=0.9 Hz, 1H), 2.44 (s, 3H). ¹³C NMR (101 MHZ, DMSO-d₆) δ166.97, 145.35, 133.15, 130.41, 129.99, 129.53, 122.31, 85.77, 81.77, 20.78.

3-ethynyl-4-methyl-N-(4-((4-methylpiperazin-1-yl)methyl)-3-(trifluoromethyl)phenyl) benzamide (19). The title compound was prepared using the general procedure that was described for the synthesis of 3c, except for using 4-((4-methylpiperazin-1-yl)methyl)-3-(trifluoromethyl)aniline 17 (1.0 g, 3.66 mmol) and 3-ethynyl-4-methylbenzoic acid 16 (0.58 g, 3.66 mmol) as the starting materials, as depicted in scheme 4. The desired compound was obtained as an off-white solid (0.9 g, 59% yield). ¹H NMR (400 MHZ, DMSO-d₆) δ11.65 (s, 1H), 10.72 (s, 1H), 8.33 (d, J=2.2 Hz, 1H), 8.17 (dd, J=8.6, 2.2 Hz, 1H), 8.11 (d, J=2.0 Hz, 1H), 7.95 (dd, J=8.0, 2.0 Hz, 1H), 7.48 (d, J=8.1 Hz, 1H), 4.53 (s, 1H), 4.22 (s, 2H), 3.46 (d, J=77.4 Hz, 8H), 2.79 (s, 3H), 2.46 (s, 3H). ¹³C NMR (101 MHZ, DMSO-d₆) δ165.32, 144.71, 132.30, 131.60, 130.34, 128.80, 123.90, 122.22, 117.87, 117.81, 85.99, 81.94, 20.70. LC-MS (ESI-QQQ): m/z 416.2 ([C₂₃H₂₄F₃N₃O+H]⁺ calcd. 416.19). Purity 99% (RT 4.273 min).

4-methyl-3-((1-methyl-1H-imidazol-4-yl)ethynyl)-N-(4-((4-methylpiperazin-1-yl)methyl)-3-(trifluoromethyl)phenyl)benzamide (20). The title compound was prepared following the general Sonogashira coupling, as described for the synthesis of 8, except for using 19 (0.25 g, 0.60 mmol) and 4-iodo-1-methyl-1H-imidazole (0.14 g, 0.66 mmol) as starting materials. Instead of Pd(PPh₃)₄, [Pd(PPh₃)₂Cl₂] was used as a catalyst. The desired compound was obtained as an off-white solid (0.05 g, 18% yield). ¹H NMR (400 MHZ, DMSO-d₆) δ10.48 (s, 1H), 8.19 (d, J=2.2 Hz, 1H), 8.09 (d, J=1.9 Hz, 1H), 8.04 (dd, J=8.5, 2.2 Hz, 1H), 7.85 (dd, J=8.0, 2.0 Hz, 1H), 7.67 (q, J=6.9, 5.2 Hz, 2H), 7.57 (d, J=1.3 Hz, 1H), 7.46 (d, J=8.1 Hz, 1H), 3.66 (s, 3H), 3.54 (s, 2H), 3.30 (s, 3H), 2.34 (d, J=22.8 Hz, 8H), 2.14 (s, 3H). ¹³C NMR (101 MHZ, DMSO-d₆) δ165.15, 143.61, 139.35, 138.64, 132.49, 131.66, 130.54, 130.32, 128.12, 127.68, 126.00, 123.94, 123.14, 122.77, 89.80, 86.50, 57.89, 55.16, 53.12, 46.14, 33.66, 20.78. LC-MS (ESI-QQQ): m/z 496.20 ([C₂₇H₂₈F₃N₅O+H]⁺ calcd. 496.22). Purity 99% (RT 3.610 min).

Compounds 21a-b were prepared from 17 and the corresponding reactant 18 by a similar method that was described for the synthesis of 3c.

4-methyl-N-(4-((4-methylpiperazin-1-yl)methyl)-3-(trifluoromethyl)phenyl)benzamide (21a). The title compound was obtained as an off-white solid (59%). ¹H NMR (400 MHZ, DMSO-d₆) δ10.39 (s, 1H), 8.19 (d, J=2.2 Hz, 1H), 8.02 (dd, J=8.5, 2.2 Hz, 1H), 7.92-7.78 (m, 2H), 7.67 (d, J=8.5 Hz, 1H), 7.41-7.24 (m, 2H), 3.54 (d, J=1.9 Hz, 2H), 2.37 (s, 8H), 2.14 (s, 3H). ¹³C NMR (101 MHZ, DMSO-d₆) δ166.03, 142.38, 138.78, 132.31, 132.01, 131.63, 129.42, 128.17, 127.96, 127.67, 126.17, 123.89, 117.65, 117.58, 57.89, 55.16, 53.11, 46.13, 21.47. LC-MS (ESI-QQQ): m/z 392.2 ([C₂₁H₂₄F₃N₃O+H]⁺ calcd. 392.19). Purity 99% (RT 4.020 min).

4-methyl-N-(4-((4-methylpiperazin-1-yl)methyl)-3-(trifluoromethyl)phenyl)-3-((4-(pyridin-3-yl)pyrimidin-2-yl)amino)benzamide (21b). The title compound was obtained as an off-white solid (10%). ¹H NMR (400 MHZ, DMSO-d₆) δ10.44 (s, 1H), 9.28 (dd, J=2.3, 0.9 Hz, 1H), 9.16 (s, 1H), 8.69 (dd, J=4.8, 1.7 Hz, 1H), 8.56 (d, J=5.1 Hz, 1H), 8.46 (ddd, J=8.0, 2.3, 1.7 Hz, 1H), 8.30 (d, J=1.9 Hz, 1H), 8.21 (d, J=2.2 Hz, 1H), 8.07 (dd, J=8.5, 2.2 Hz, 1H), 7.79-7.61 (m, 2H), 7.56-7.46 (m, 2H), 7.44 (dd, J=7.9, 0.8 Hz, 1H), 3.57 (d, J=1.6 Hz, 2H), 2.38 (d, J=13.7 Hz, 12H), 2.18 (s, 3H). ¹³C NMR (101 MHZ, DMSO-d₆) δ165.83, 162.02, 161.47, 160.02, 151.89, 148.57, 138.75, 138.52, 137.00, 134.71, 131.63, 130.76, 124.73, 124.24, 123.95, 108.34, 57.87, 55.13, 53.08, 46.10, 40.57, 40.36, 40.16, 39.95, 39.84, 39.74, 39.53, 39.32, 18.68. LC-MS (ESI-QQQ): m/z 562.30 ([C₃₀H₃₀F₃N₇O+H]⁺ calcd. 562.25). Purity 97.7% (RT 3.863 min).

3-iodo-4-methoxy-N-(4-((4-methylpiperazin-1-yl)methyl)-3-(trifluoromethyl)phenyl)benzamide (23). The title compound was prepared using a similar method that was described for the synthesis of 3c, except for using 17 (0.5 g, 1.83 mmol) and 3-iodo-4-methoxybenzoic acid 22 (0.53 g, 1.92 mmol) as the starting materials as shown in scheme-4. The desired product was obtained as an off-white solid (0.86 g, 89% yield). ¹H NMR (400 MHZ, DMSO-d₆) δ10.40 (s, 1H), 8.42 (d, J=2.2 Hz, 1H), 8.17 (d, J=2.2 Hz, 1H), 8.03 (dt, J=8.6, 2.5 Hz, 2H), 7.69 (d, J=8.5 Hz, 1H), 7.15 (d, J=8.7 Hz, 1H), 3.92 (s, 3H), 3.55 (s, 2H), 2.36 (d, J=20.7 Hz, 8H), 2.15 (s, 3H). ¹³C NMR (101 MHz, DMSO-d₆) δ164.16, 160.95, 138.72, 138.67, 132.38, 131.63, 130.50, 128.58, 127.95, 127.65, 126.16, 123.92, 117.69, 117.63, 111.46, 86.27, 57.89, 57.23, 55.16, 53.13, 46.15, 31.40, 22.51, 14.40. LC-MS (ESI-QQQ): m/z 534.1 ([C₂₁H₂₃F₃IN₃O₂+H]⁺ calcd. 534.08). Purity 94.2% (RT 4.237 min).

3-(imidazo[1,2-b]pyridazin-3-ylethynyl)-4-methoxy-N-(4-((4-methylpiperazin-1-yl)methyl)-3-(trifluoromethyl)phenyl)benzamide (24). The title compound was prepared following the general Sonogashira coupling, as described for the synthesis of 8, except for using 23 (0.2 g, 0.37 mmol) and 5 (0.05 g, 0.37 mmol) as the starting material as shown in scheme 4. The desired product was obtained as an off-white solid (0.04 g, 20% yield). ¹H NMR (400 MHZ, DMSO-d₆) δ10.49 (s, 1H), 8.71 (dd, J=4.5, 1.4 Hz, 1H), 8.39-8.14 (m, 4H), 8.07 (td, J=9.2, 2.2 Hz, 2H), 7.70 (d, J=8.6 Hz, 1H), 7.39 (dd, J=9.2, 4.5 Hz, 1H), 7.31 (d, J=8.9 Hz, 1H), 3.98 (s, 3H), 3.57 (s, 2H), 2.46 (d, J=26.4 Hz, 7H), 2.23 (s, 3H). ¹³C NMR (101 MHZ, DMSO-d₆) δ164.65, 162.57, 145.35, 139.95, 138.76, 138.65, 132.80, 131.68, 131.54, 126.91, 126.51, 123.92, 119.42, 112.32, 111.95, 110.99, 94.54, 81.03, 57.78, 56.79, 54.92. LC-MS (ESI-QQQ): m/z 549.30 ([C₂₉H₂₇F₃N₆O₂+H]⁺ calcd. 549.21). Purity 99% (RT 3.943 min).

3-ethynyl-4-methyl-N-(3-(4-methyl-1H-imidazol-1-yl)-5-(trifluoromethyl)phenyl)benzamide (26). The title compound was prepared using a similar method that was described for the synthesis of 3c, except for using 3-(4-methyl-1H-imidazol-1-yl)-5-(trifluoromethyl)aniline 25 (1.0 g, 4.14 mmol) and 3-ethynyl-4-methylbenzoic acid 16 (0.67 g, 4.14 mmol) as the starting materials as depicted in scheme 5. The desired compound was obtained as an off-white solid (0.4 g, 25% yield). ¹H NMR (400 MHZ, DMSO-d₆) δ10.65 (s, 1H), 8.26 (s, 1H), 8.18 (s, 1H), 8.15-8.07 (m, 2H), 7.91 (dd, J=8.1, 1.8 Hz, 1H), 7.71 (s, 1H), 7.63-7.53 (m, 1H), 7.48 (t, J=5.8 Hz, 1H), 4.53 (s, 1H), 2.45 (s, 3H), 2.16 (s, 3H). ¹³C NMR (101 MHZ, DMSO-d₆) δ165.31, 144.87, 141.65, 138.38, 132.14, 131.98, 131.88, 131.51, 131.45, 131.13, 130.45, 129.26, 129.15, 128.76, 122.28, 115.40, 114.68, 86.04, 81.91, 20.71, 14.02. LC-MS (ESI-QQQ): m/z 384.10 ([C₂₁H₁₆F₃N₃O+H]⁺ calcd. 384.12). Purity 89.6% (RT 4.510 min).

3-ethynyl-4-methoxybenzoic acid (28). The compound 28 was prepared according to the general procedure described for the synthesis of 16 except for using methyl 3-iodo-4-methoxybenzoate 27 (1.0 g, 3.42 mmol) as the starting material. The desired product was obtained as an off-white solid (0.19 g, 32% yield). ¹H NMR (400 MHZ, DMSO-d₆) δ12.83 (s, 1H), 7.95 (dd, J=8.7, 2.3 Hz, 1H), 7.90 (d, J=2.1 Hz, 1H), 7.17 (d, J=8.8 Hz, 1H), 4.31 (d, J=0.8 Hz, 1H), 3.90 (s, 3H). ¹³C NMR (101 MHZ, DMSO-d₆) δ166.67, 163.89, 134.99, 132.45, 123.40, 111.77, 111.26, 85.58, 79.53, 56.59.

3-ethynyl-4-methoxy-N-(4-((4-methylpiperazin-1-yl)methyl)-3-(trifluoromethyl)phenyl)benzamide (29). The title compound was prepared using a similar method that was described for the synthesis of 3c, except for using 28 (0.19 g, 1.10 mmol) and 17 (0.3 g, 1.10 mmol) as the starting materials as shown in scheme 5. The title compound was obtained as an off-white solid (0.15 g, 32% yield). ¹H NMR (400 MHZ, DMSO-d₆) δ10.43 (s, 1H), 8.20 (d, J=2.2 Hz, 1H), 8.12 (d, J=2.3 Hz, 1H), 8.05 (ddd, J=8.4, 5.1, 2.2 Hz, 2H), 7.70 (d, J=8.6 Hz, 1H), 7.24 (d, J=8.8 Hz, 1H), 4.40 (s, 1H), 3.93 (s, 3H), 3.58 (s, 2H), 2.51-2.29 (m, 8H), 2.23 (s, 3H). ¹³C NMR (101 MHZ, DMSO-d₆) δ164.65, 163.21, 138.74, 133.28, 131.66, 131.08, 126.68, 111.71, 110.99, 85.67, 79.82, 57.78, 56.58, 54.95, 52.76. LC-MS (ESI-QQQ): m/z 432.2 ([C₂₃H₂₄F₃N₃O₂+H]⁺ calcd. 432.18). Purity 99% (RT 3.950 min).

N-(3-iodo-4-methylphenyl)-4-((4-methylpiperazin-1-yl)methyl)benzamide (31). The title compound was prepared using a similar method that was described for the synthesis of 3c, except for using 4-((4-methylpiperazin-1-yl)methyl)benzoic acid 2d (0.5 g, 2.14 mmol) and 3-iodo-4-methylaniline 30 (0.6 g, 2.56 mmol) as the starting materials as shown in scheme 5. The title compound was obtained as an off-white solid (0.72 g, 75% yield). 1H NMR (400 MHZ, CDCl₃) δ8.11 (d, J=2.2 Hz, 1H), 7.81 (dd, J=8.1, 6.4 Hz, 3H), 7.57 (dd, J=8.2, 2.3 Hz, 1H), 7.48-7.37 (m, 2H), 7.20 (dd, J=8.2, 0.8 Hz, 1H), 3.57 (s, 2H), 2.54 (s, 8H), 2.41 (s, 3H), 2.35 (s, 3H). ¹³C NMR (101 MHZ, CDCl₃) δ165.37, 142.59, 137.51, 136.49, 133.42, 130.19, 129.58, 129.34, 127.04, 120.12, 100.62, 62.32, 54.91, 52.63, 45.68, 27.38. LC-MS (ESI-QQQ): m/z 450.1 ([C₂₀H₂₄ IN₃O+H]⁺ calcd. 450.19). Purity 99% (RT 3.843 min).

N-(3-(imidazo[1,2-b]pyridazin-3-ylethynyl)-4-methylphenyl)-4-((4-methylpiperazin-1-yl)methyl)benzamide (32). The title compound was prepared following the general Sonogashira coupling, as described for the synthesis of 8, except for using 31 (0.34 g, 0.768 mmol) and 5 (0.1 g, 0.70 mmol) as the starting materials as shown in scheme 5. The title compound was obtained as an off-white solid (0.062 g, 19% yields). ¹H NMR (400 MHZ, DMSO-d₆) δ10.25 (s, 1H), 8.69 (dd, J=4.5, 1.6 Hz, 1H), 8.28-8.16 (m, 2H), 8.06 (d, J=2.2 Hz, 1H), 7.94-7.86 (m, 2H), 7.70 (dd, J=8.3, 2.3 Hz, 1H), 7.45-7.40 (m, 2H), 7.36 (dd, J=9.2, 4.4 Hz, 1H), 7.33-7.30 (m, 1H), 3.52 (s, 3H), 3.30 (s, 2H), 2.37 (bs, 8H), 2.17 (s, 3H). ¹³C NMR (101 MHZ, DMSO-d₆) δ165.85, 145.44, 142.75, 139.98, 138.50, 137.65, 134.91, 133.85, 130.45, 129.13, 128.08, 126.51, 122.98, 122.01, 121.57, 119.35, 112.38, 97.56, 80.52, 61.98, 55.03, 52.83, 45.98, 20.17. LC-MS (ESI-QQQ): m/z 465.40 ([C₂₈H₂₈N₆O+H]⁺ calcd. 465.23). Purity 99% (RT 3.673 min).

General procedure for the synthesis of 33 a-h. The following procedure is for N-(3-(1H-imidazol-1-yl)-5-(trifluoromethyl)phenyl)-3-(imidazo[1,2-b]pyridazin-3-ylethynyl)-4-methylbenzamide (33a). Compound 33a was prepared according to the previously reported methods for similar compounds, 44-45 with several modifications. N-(3-bromo-5-(trifluoromethyl)phenyl)-3-(imidazo[1,2-b]pyridazin-3-ylethynyl)-4-methylbenzamide 15 (3.0 g, 6.00 mmol) and ¹H-imidazole (0.45 g, 6.61 mmol) were taken in dry DMSO (50 mL) in a pressure tube. The solution was purged with a nitrogen flow for 10 min then Cul (0.17 g, 0.90 mmol), K₂CO₃ (2.5 g. 18.0 mmol), and 8-hydroxyquinoline(0.13 g, 0.90 mmol) were added and purging was continued for another 10 min. The pressure tube was then sealed tightly and stirred at 100° C. for 18 h. Upon cooling to rt, the reaction mixture was poured into ice-cold water (˜50 mL) and allowed to stir for 30 min, during which time pale yellow solid was observed. The solid was collected by filtration and then dissolved in 10% MeOH in DCM (100 mL). The undissolved solid was removed by filtration. The filtrate was evaporated to dryness to afford crude product, which was purified on a silica gel column using a 0-10% gradient of methanol in DCM as an eluent to obtain the desired product as a pale yellow solid (1.67 g, 57% yield). ¹H NMR (400 MHZ, DMSO-d₆) δ10.78 (s, 1H), 8.73 (dt, J=4.5, 1.4 Hz, 1H), 8.34 (s, 2H), 8.30-8.19 (m, 4H), 7.98 (dd, J=8.1, 1.9 Hz, 1H), 7.81 (d, J=9.6 Hz, 2H), 7.59 (d, J=8.1 Hz, 1H), 7.40 (ddd, J=9.2, 4.5, 1.1 Hz, 1H), 7.18 (s, 1H), 2.63 (s, 3H). ¹³C NMR (101 MHZ, DMSO-d₆) δ165.32, 145.54, 144.33, 141.68, 138.75, 132.29, 130.70, 130.59, 129.02, 126.58, 122.33, 119.58, 116.04, 96.77, 81.72, 20.89. LC-MS (ESI-QQQ): m/z 487.20 ([C₂₆H₁₇F₃N₆O+H]⁺ calcd. 487.14). Purity 99% (RT 4.510 min).

N-(3-(4-ethyl-1H-imidazol-1-yl)-5-(trifluoromethyl)phenyl)-3-(imidazo[ 1,2-b]pyridazin-3-ylethynyl)-4-methylbenzamide (33b). Compound 33b was synthesized from 15 (0.1 g, 0.20 mmol) and 4-ethyl-1H-imidazole (0.03 g, 0.30 mmol) according to the general procedure for the synthesis of 33 a-h. After completion of the reaction, the reaction mixture was cooled to rt and 10% NH₄OH solution was added. The product was extracted into EtOAc (3×25 mL). The combined organic layers were washed with water followed by 10% NH₄OH solution, dried over Na₂SO₄, filtered and evaporated to dryness. The crude product was purified on a silica gel column using a 0-10% gradient of methanol in DCM as an eluent to yield the title compound as a pale yellow solid (3.0 mg, 3% yield). ¹H NMR (400 MHZ, DMSO-d₆) δ10.74 (s, 1H), 8.73 (dd, J=4.4, 1.6 Hz, 1H), 8.34-8.17 (m, 6H), 7.98 (dd, J=7.9, 2.0 Hz, 1H), 7.77 (q, J=1.8, 1.3 Hz, 1H), 7.62-7.56 (m, 1H), 7.51 (s, 1H), 7.40 (dd, J=9.2, 4.5 Hz, 1H), 2.63 (s, 3H), 2.60-2.54 (m, 2H), 1.21 (d, J=7.5 Hz, 3H). LC-MS (ESI-QQQ): m/z 515.30 ([C₂₈H₂₁F₃N₆O+H]⁺ calcd. 515.17). Purity 87.5% (RT 4.663 min).

3-(imidazo[1,2-b]pyridazin-3-ylethynyl)-N-(3-(4-isopropyl-1H-imidazol-1-yl)-5-(trifluoromethyl)phenyl)-4-methylbenzamide (33c). Compound 33c was prepared from 15 (0.1 g, 0.20 mmol) and 4-isopropyl-1H-imidazole (0.03 g, 2.40 mmol) using a similar method that was described for the synthesis of 33b. The desired product was obtained as a pale yellow solid (0.03 g, 29% Yield). ¹H NMR (400 MHZ, DMSO-d₆) δ10.74 (s, 1H), 8.74 (dt, J=4.4, 1.5 Hz, 1H), 8.36-8.14 (m, 6H), 7.98 (dt, J=8.0, 1.8 Hz, 1H), 7.81-7.73 (m, 1H), 7.59 (d, J=8.0 Hz, 1H), 7.50 (s, 1H), 7.40 (ddd, J=9.2, 4.5, 1.3 Hz, 1H), 2.85 (p, J=6.7 Hz, 1H), 2.63 (d, J=1.3 Hz, 3H), 1.24 (dd, J=6.8, 1.4 Hz, 6H). ¹³C NMR (101 MHZ, DMSO-d₆) δ165.28, 145.54, 144.31, 141.62, 138.75, 138.45, 132.30, 130.69, 130.57, 129.00, 126.58, 122.33, 119.57, 115.45, 96.77, 81.71, 27.80, 22.62, 20.88. LC-MS (ESI-QQQ): m/z 529.2 ([C₂₉H₂₃F₃N₆O+H]⁺ calcd. 529.19). Purity 99% (RT 4.847 min).

N-(3-(4-cyclopropyl-1H-imidazol-1-yl)-5-(trifluoromethyl)phenyl)-3-(imidazo[1,2-b]pyridazin-3-ylethynyl)-4-methylbenzamide (33d). Compound 33d was prepared from 15 (0.03 g, 0.06 mmol) and 4-cyclopropyl-1H-imidazole (8.0 mg, 0.07 mmol) using a similar method that was described for the synthesis of 33b. The desired product was obtained as a pale yellow solid (20.0 mg, 29% Yield). ¹H NMR (400 MHZ, DMSO-d₆) δ10.75 (s, 1H), 8.73 (dd, J=4.5, 1.6 Hz, 1H), 8.32-8.27 (m, 2H), 8.27-8.23 (m, 2H), 8.17 (s, 2H), 7.98 (dd, J=7.9, 2.0 Hz, 1H), 7.75 (s, 1H), 7.63-7.57 (m, 1H), 7.55 (s, 1H), 7.40 (dd, J=9.2, 4.5 Hz, 1H), 2.62 (s, 3H), 1.92-1.79 (m, 1H), 0.87-0.79 (m, 2H), 0.76-0.66 (m, 2H). LC-MS (ESI-QQQ): m/z 527.30 ([C₂₉H₂₁F₃N₆O+H]⁺ calcd. 527.17). Purity 99% (RT 4.910 min).

3-(imidazo[1,2-b]pyridazin-3-ylethynyl)-4-methyl-N-(3-(3-methyl-1H-pyrrol-1-yl)-5-(trifluoromethyl)phenyl)benzamide (33e). Compound 33e was prepared from 15 (0.1 g, 0.20 mmol) and 3-methyl-1H-pyrrole (0.02 g, 0.30 mmol) using a similar method that was described for the synthesis of 33b. The desired product was obtained as a pale yellow solid (4.0 mg, 4% Yield). ¹H NMR (400 MHz, DMSO-d₆) δ10.66 (s, 1H), 8.73 (dd, J=4.5, 1.5 Hz, 1H), 8.29-8.20 (m, 4H), 8.08 (d, J=1.9 Hz, 1H), 7.97 (dd, J=8.0, 2.0 Hz, 1H), 7.62-7.55 (m, 2H), 7.40 (dd, J=9.2, 4.5 Hz, 1H), 7.34 (t, J=2.6 Hz, 1H), 7.21 (p, J=1.3 Hz, 1H), 6.18 (dd, J=2.9, 1.7 Hz, 1H), 2.62 (s, 3H), 2.10 (d, J=1.0 Hz, 3H). LC-MS (ESI-QQQ): m/z 500.30 ([C₂₈H₂₀F₃N₅O+H]⁺ calcd. 500.16). Purity 76.6% (RT 6.180 min).

3-(imidazo[1,2-b]pyridazin-3-ylethynyl)-4-methyl-N-(3-(4-methylpiperazin-1-yl)-5-(trifluoromethyl)phenyl)benzamide (33f). Compound 33f was prepared from 15 (0.1 g, 0.20 mmol) and 1-methylpiperazine (0.03 g, 0.30 mmol) using a similar method that was described for the synthesis of 33b. The desired product was obtained as a pale yellow solid (3.0 mg, 4% Yield). ¹H NMR (400 MHZ, DMSO-d₆) δ10.39 (s, 1H), 8.73 (d, J=4.4 Hz, 1H), 8.24 (dd, J=17.4, 10.6 Hz, 3H), 7.94 (d, J=8.1 Hz, 1H), 7.67 (d, J=19.8 Hz, 2H), 7.55 (d, J=8.1 Hz, 1H), 7.40 (dd, J=9.3, 4.5 Hz, 1H), 6.96 (s, 1H), 3.26 (d, J=26.8 Hz, 8H), 2.61 (s, 3H), 2.24 (s, 3H). LC-MS (ESI-QQQ): m/z 519.30 ([C₂₈H₂₅F₃N₆O+H]⁺ calcd. 519.20). Purity 93.5% (RT 4.247 min).

N-(3-(1H-benzo[d]imidazol-1-yl)-5-(trifluoromethyl)phenyl)-3-(imidazo[1,2-b]pyridazin-3-ylethynyl)-4-methylbenzamide (33g). Compound 33g was prepared from 15 (0.1 g, 0.20 mmol) and 1H-benzo[d]imidazole (0.03 g, 0.24 mmol) using a similar method that was described for the synthesis of 33b. The desired product was obtained as a pale yellow solid (15.0 mg, 14% Yield). ¹H NMR (400 MHZ, DMSO-d₆) δ10.87 (d, J=3.2 Hz, 1H), 8.81-8.61 (m, 2H), 8.49 (bs, 1H), 8.36 (bs, 1H), 8.26 (q, J=3.8, 3.2 Hz, 3H), 8.00 (dd, J=8.3, 3.1 Hz, 1H), 7.88-7.71 (m, 3H), 7.59 (dd, J=8.3, 3.2 Hz, 1H), 7.48-7.28 (m, 3H), 2.63 (s, 3H). ¹³C NMR (101 MHZ, DMSO-d₆) δ165.47, 145.54, 144.35, 141.78, 138.76, 137.53, 132.34, 130.69, 130.63, 129.07, 126.58, 124.28, 123.33, 122.33, 120.64, 119.58, 118.48, 111.16, 96.77, 81.75, 20.89. LC-MS (ESI-QQQ): m/z 537.20 ([C₃₀H₁₉F₃N₆O+H]⁺ calcd. 537.16). Purity 92% (RT 5.613 min).

3-(imidazo[1,2-b]pyridazin-3-ylethynyl)-4-methyl-N-(3-(4-phenyl-1H-imidazol-1-yl)-5-(trifluoromethyl)phenyl)benzamide (33h): Compound 33h was prepared from 15 (0.1 g, 0.20 mmol) and 4-phenyl-1H-imidazole (0.03 g, 0.24 mmol) using a similar method that was described for the synthesis of 33b. The desired product was obtained as a pale yellow solid (0.04 g, 36% Yield). ¹H NMR (400 MHZ, DMSO-d₆) δ10.81 (s, 1H), 8.79-8.68 (m, 1H), 8.47-8.33 (m, 3H), 8.31-8.18 (m, 4H), 8.00 (dd, J=8.0, 2.0 Hz, 1H), 7.94-7.83 (m, 3H), 7.60 (d, J=8.0 Hz, 1H), 7.49-7.34 (m, 3H), 7.28 (d, J=7.2 Hz, 1H), 2.63 (s, 3H). ¹³C NMR (101 MHZ, DMSO-d₆) δ165.30, 145.54, 144.34, 142.67, 141.69, 140.14, 138.75, 138.22, 136.59, 134.16, 132.26, 130.70, 130.60, 129.06, 129.02, 127.37, 126.58, 125.01, 122.35, 119.57, 115.83, 114.62, 96.77, 81.72, 20.89. LC-MS (ESI-QQQ): m/z 563.2 ([C₃₂H₂₁F₃N₆O+H]⁺ calcd. 563.17). Purity 99% (RT 5.793 min).

3-iodo-4-methyl-N-(3-(4-methyl-1H-imidazol-1-yl)-5-(trifluoromethyl)phenyl)benzamide (35a). The title compound was synthesized following the procedure described for the synthesis of 14 except for using 3-(4-methyl-1H-imidazol-1-yl)-5-(trifluoromethyl)aniline 25 (2.0 g, 8.3 mmol) and 3-iodo-4-methylbenzoic acid 34a (2.5 g, 9.12 mmol) as the starting materials as depicted in scheme 7. The desired product was obtained as an off-white solid (3.83 g, 95% yield). ¹H NMR (400 MHZ, DMSO-d₆) δ10.68 (s, 1H), 8.45 (s, 1H), 8.27 (s, 1H), 8.20 (s, 1H), 8.13 (s, 1H), 7.93 (d, J=8.0 Hz, 1H), 7.72 (s, 1H), 7.55-7.40 (m, 2H), 2.44 (s, 3H), 2.18 (d, J=2.4 Hz, 3H). ¹³C NMR (101 MHZ, DMSO-d₆) δ164.58, 145.82, 141.61, 139.39, 138.36, 137.94, 135.41, 133.60, 131.45, 131.12, 130.34, 128.32, 125.46, 122.75, 115.39, 114.64, 112.15, 112.11, 101.63, 28.05, 14.03. LC-MS (ESI-QQQ): m/z 486.00 ([C₁₉H₁₅F₃IN₃O+H]⁺ calcd. 486.02). Purity 97.5% (RT 4.773 min).

3-iodo-N-(3-(4-methyl-1H-imidazol-1-yl)-5-(trifluoromethyl)phenyl)benzamide (35b). The title compound was synthesized following the procedure described for the synthesis of 14 except for using 3-(4-methyl-1H-imidazol-1-yl)-5-(trifluoromethyl)aniline 25 (2.43 g, 10.07 mmol) and 3-iodobenzoic acid 34b (2.5 g, 10.07 mmol) as the starting materials as depicted in scheme 7. The desired product was obtained as an off-white solid (4.06 g, 86% yield). ¹H NMR (400 MHZ, DMSO-d₆) δ10.73 (s, 1H), 8.35 (q, J=1.8 Hz, 1H), 8.28-8.07 (m, 3H), 8.00 (dq, J=7.8, 1.3 Hz, 2H), 7.74 (d, J=1.9 Hz, 1H), 7.48 (q, J=1.2 Hz, 1H), 7.38 (td, J=7.8, 1.5 Hz, 1H), 2.22-2.13 (m, 3H). ¹³C NMR (101 MHZ, DMSO-d₆) δ164.91, 141.50, 141.10, 139.39, 138.39, 136.49, 136.39, 135.41, 131.46, 131.18, 127.75, 115.44, 114.79, 114.75, 114.63, 112.28, 95.28, 14.01. LC-MS (ESI-QQQ): m/z 472.00 ([C₁₈H₁₃F₃IN₃O+H]⁺ calcd. 472.01). Purity 99% (RT 4.457 min).

3-iodo-4-methoxy-N-(3-(4-methyl-1H-imidazol-1-yl)-5-(trifluoromethyl)phenyl)benzamide (35c). The title compound was synthesized following the procedure described for the synthesis of 14 except for using 3-(4-methyl-1H-imidazol-1-yl)-5-(trifluoromethyl)aniline 25 (2.0 g, 7.2 mmol) and 3-iodo-4-methoxybenzoic acid 34c (1.73 g, 7.2 mmol) as the starting materials as depicted in scheme 7. The desired product was obtained as an off-white solid (2.63 g, 73% yield). ¹H NMR (400 MHZ, DMSO-d₆) δ10.57 (s, 1H), 8.46 (dd, J=2.3, 1.2 Hz, 1H), 8.27 (t, J=1.9 Hz, 1H), 8.20 (t, J=1.4 Hz, 1H), 8.13 (d, J=1.8 Hz, 1H), 8.06 (ddd, J=8.6, 2.3, 1.2 Hz, 1H), 7.74-7.67 (m, 1H), 7.47 (d, J=1.5 Hz, 1H), 7.17 (dd, J=8.8, 1.3 Hz, 1H), 3.93 (s, 3H), 2.18 (s, 3H). ¹³C NMR (101 MHZ, DMSO-d₆) δ164.41, 161.18, 141.74, 139.36, 138.74, 138.34, 135.40, 131.40, 131.08, 130.62, 128.16, 125.45, 122.74, 115.34, 114.64, 111.97, 111.56, 86.35, 57.26, 14.01. LC-MS (ESI-QQQ): m/z 502.00 ([C₁₉H₁₅F₃IN₃O₂+H]⁺ calcd. 502.02). Purity 99% (RT 4.450 min).

Compounds 36a-c were prepared from 5 and the corresponding reactant 35 by a similar method that was described for the synthesis of 8.

3-(imidazo[1,2-b]pyridazin-3-ylethynyl)-4-methyl-N-(3-(4-methyl-1H-imidazol-1-yl)-5-(trifluoromethyl)phenyl)benzamide (36a). The title compound was obtained as a pale yellow solid (49% yield). ¹H NMR (400 MHZ, DMSO-d₆) δ10.72 (s, 1H), 8.71 (d, J=4.4 Hz, 1H), 8.39-8.06 (m, 6H), 7.99-7.89 (m, 1H), 7.72 (s, 1H), 7.56 (d, J=8.1 Hz, 1H), 7.48 (s, 1H), 7.38 (dd, J=9.3, 4.4 Hz, 1H), 2.60 (s, 3H), 2.16 (s, 3H). ¹³C NMR (101 MHZ, DMSO-d₆) δ165.29, 145.53, 144.31, 141.66, 138.74, 138.39, 132.30, 130.68, 130.58, 129.00, 126.57, 122.32, 119.57, 115.43, 112.15, 110.00, 96.77, 81.71, 20.88, 14.02. LC-MS (ESI-QQQ): m/z 501.30 ([C₂₇H₁₉F₃N₆O+H]⁺ calcd. 501.16). Purity 99% (RT 4.427 min).

3-(imidazo[1,2-b]pyridazin-3-ylethynyl)-N-(3-(4-methyl-1H-imidazol-1-yl)-5-(trifluoromethyl)phenyl)benzamide (36b). The title compound was obtained as a pale yellow solid (61% yield). ¹H NMR (400 MHZ, DMSO-d₆) δ10.81 (s, 1H), 8.72 (dd, J=4.4, 1.6 Hz, 1H), 8.33 -8.20 (m, 5H), 8.17 (d, J=1.7 Hz, 1H), 8.05 (dt, J=7.9, 1.5 Hz, 1H), 7.86 (dt, J=7.7, 1.4 Hz, 1H), 7.75 (t, J=1.8 Hz, 1H), 7.67 (t, J=7.8 Hz, 1H), 7.49 (s, 1H), 7.40 (dd, J=9.2, 4.4 Hz, 1H), 2.18 (s, 3H). ¹³C NMR (101 MHZ, DMSO-d₆) δ165.44, 145.46, 141.60, 140.13, 139.43, 139.00, 138.42, 135.10, 134.93, 131.50, 131.18, 130.46, 129.92, 129.12, 126.60, 125.46, 122.75, 122.48, 119.62, 115.46, 114.71, 112.30, 111.98, 97.78, 78.08, 14.04. LC-MS (ESI-QQQ): m/z 487.20 ([C₂₆H₁₇F₃N₆O+H]⁺ calcd. 487.14). Purity 99% (RT 4.197 min).

3-(imidazo[1,2-b]pyridazin-3-ylethynyl)-4-methoxy-N-(3-(4-methyl-1H-imidazol-1-yl)-5-(trifluoromethyl)phenyl)benzamide (36c). The title compound was obtained as a pale yellow solid (55% yield). ¹H NMR (400 MHZ, DMSO-d₆) δ10.66 (s, 1H), 8.71 (dt, J=4.5, 1.3 Hz, 1H), 8.32 -8.18 (m, 5H), 8.16 (s, 1H), 8.11 (ddd, J=8.8, 2.5, 1.0 Hz, 1H), 7.73 (s, 1H), 7.51 (s, 1H), 7.39 (ddd, J=9.2, 4.4, 1.1 Hz, 1H), 7.34 (dd, J=9.0, 1.1 Hz, 1H), 3.99 (d, J=1.0 Hz, 3H), 2.18 (s, 3H). ¹³C NMR (101 MHZ, DMSO-d₆) δ164.92, 162.79, 145.38, 141.80, 138.71, 138.40, 132.82, 131.65, 131.43, 131.11, 126.54, 125.46, 122.75, 119.46, 115.39, 114.72, 112.11, 111.10, 94.46, 81.14, 56.84, 14.04. LC-MS (ESI-QQQ): m/z 517.20 ([C₂₇H₁₉F₃N₆O₂+H]⁺ calcd. 517.17). Purity 99% (RT 4.137 min).

Compounds 37a-b were prepared from 13 and the corresponding reactant 34 by a similar method that was described for the synthesis of 14.

N-(3-bromo-5-(trifluoromethyl)phenyl)-3-iodobenzamide (37a). The title compound was obtained as an off-white solid (76% yield). 1H NMR (400 MHZ, DMSO-d₆) δ10.68 (s, 1H), 8.34 (dd, J=6.4, 1.8 Hz, 2H), 8.23-8.14 (m, 1H), 7.98 (ddt, J=7.9, 5.6, 1.4 Hz, 2H), 7.73-7.62 (m, 1H), 7.36 (td, J=7.8, 1.4 Hz, 1H). ¹³C NMR (101 MHz, DMSO-d₆) δ164.92, 141.64, 141.10, 136.42, 136.40, 131.72, 131.40, 131.13, 127.76, 126.47, 124.93, 123.11, 123.07, 122.66, 122.22, 115.91, 115.87, 95.24. LC-MS (ESI-QQQ): m/z 469.8 ([C₁₄H₈BrF₃INO+H]⁺ calcd. 469.88). Purity 99% (RT 6.240 min).

N-(3-bromo-5-(trifluoromethyl)phenyl)-3-iodo-4-methoxybenzamide (37b). The title compound was obtained as an off-white solid (78% yield). 1H NMR (400 MHZ, DMSO-d₆) δ10.50 (s, 1H), 8.45-8.39 (m, 1H), 8.34 (d, J=2.2 Hz, 1H), 8.19 (d, J=2.2 Hz, 1H), 8.07-7.97 (m, 1H), 7.67-7.58 (m, 1H), 7.14 (d, J=8.8 Hz, 1H), 3.92 (s, 3H). ¹³C NMR (101 MHZ, DMSO-d₆) δ164.38, 161.17, 141.89, 138.77, 131.66, 131.34, 130.63, 128.05, 126.32, 124.96, 122.59, 115.78, 115.74, 111.47, 86.31, 57.24. LC-MS (ESI-QQQ): m/z 599.9 ([C₁₅H₁₀BrF₃INO₂+H]⁺ calcd. 499.89). Purity 98.8% (RT 6.197 min).

Compounds 38a-b were prepared from 5 and the corresponding reactant 37 by a similar method that was described for the synthesis of 8.

N-(3-bromo-5-(trifluoromethyl)phenyl)-3-(imidazo[1,2-b]pyridazin-3-ylethynyl)benzamide (38a). The title compound was obtained as a rust colored solid (63% yield). ¹H NMR (400 MHZ, DMSO-d₆) δ10.79 (s, 1H), 8.73 (dd, J=4.5, 1.6 Hz, 1H), 8.39 (s, 1H), 8.34-8.19 (m, 4H), 8.10-7.99 (m, 1H), 7.91-7.83 (m, 1H), 7.74-7.63 (m, 2H), 7.41 (dd, J=9.2, 4.4 Hz, 1H). ¹³C NMR (101 MHZ, DMSO-d₆) δ165.49, 145.49, 141.75, 140.13, 139.00, 135.05, 134.95, 131.77, 131.45, 130.53, 129.91, 129.15, 126.61, 126.53, 123.13, 122.72, 122.46, 119.65, 115.97, 111.97, 97.77, 78.07. LC-MS (ESI-QQQ): m/z 485.00 ([C₂₂H12BrF₃N₄O+H]⁺ calcd. 485.01). Purity 89.2% (RT 5.850 min).

N-(3-bromo-5-(trifluoromethyl)phenyl)-3-(imidazo[1,2-b]pyridazin-3-ylethynyl)-4-methoxybenzamide (38b). The title compound was obtained as an off-white solid (39% yield). LC-MS (ESI-QQQ): m/z 515.00 ([C₂₃H₁₄BrF₃N₄O₂+H]⁺ calcd. 515.03). Purity 90% (RT 5.763 min).

Compounds 40a-c were prepared following the general procedure described for 33a-h. N-(3-(1H-imidazol-1-yl)-5-(trifluoromethyl)phenyl)-3-(imidazo[1,2-b]pyridazin-3-ylethynyl)benzamide (40a). Compound 40a was prepared from 38a (0.1 g, 0.20 mmol) and 1H-imidazole (15 mg, 0.22 mmol) as depicted in scheme 8. The desired product was obtained as a pale yellow solid (14 mg, 14% yield). ¹H NMR (400 MHZ, DMSO-d₆) δ10.86 (s, 1H), 8.73 (d, J=4.5 Hz, 1H), 8.33 (s, 2H), 8.26 (dd, J=15.8, 6.3 Hz, 4H), 8.06 (d, J=7.8 Hz, 1H), 7.88 (d, J=7.8 Hz, 1H), 7.82 (s, 2H), 7.69 (t, J=7.8 Hz, 1H), 7.41 (dd, J=9.3, 4.4 Hz, 1H), 7.18 (s, 1H). LC-MS (ESI-QQQ): m/z 473.20 ([C₂₅H₁₅F₃N₆O+H]⁺ calcd. 473.13). Purity 99% (RT 4.250 min).

N-(3-(4-cyclopropyl-1H-imidazol-1-yl)-5-(trifluoromethyl)phenyl)-3-(imidazo[1,2-b]pyridazin-3-ylethynyl)-4-methoxybenzamide (40b). Compound 40b was prepared from 38b (85 mg, 0.16 mmol) and 4-cyclopropyl-1H-imidazole (21 mg, 0.20 mmol) as depicted in scheme 8. The desired product was obtained as a pale yellow solid (20 mg, 22% yield). ¹H NMR (400 MHZ, DMSO-d₆) δ10.64 (s, 1H), 8.69 (d, J=4.4 Hz, 1H), 8.29-8.16 (m, 4H), 8.14 (s, 2H), 8.09 (d, J=8.8 Hz, 1H), 7.71 (s, 1H), 7.52 (s, 1H), 7.41-7.27 (m, 2H), 3.97 (d, J=1.8 Hz, 3H), 1.85 (s, 1H), 0.80 (d, J=8.1 Hz, 2H), 0.73-0.65 (m, 2H). ¹H NMR (400 MHZ, DMSO) δ10.66 (s, 1H), 8.71 (dd, J=4.4, 1.5 Hz, 1H), 8.32-8.19 (m, 4H), 8.16 (t, J=1.8 Hz, 2H), 8.11 (dd, J=8.8, 2.4 Hz, 1H), 7.73 (q, J=1.8, 1.4 Hz, 1H), 7.54 (s, 1H), 7.39 (dd, J=9.2, 4.4 Hz, 1H), 7.34 (d, J=8.9 Hz, 1H), 3.99 (s, 3H), 1.87 (qd, J=8.3, 7.8, 3.6 Hz, 1H), 0.89-0.76 (m, 2H), 0.71 (dd, J=5.0, 2.2 Hz, 2H). ¹³C NMR (101 MHZ, DMSO-d₆) δ164.94, 162.82, 145.41, 141.79, 138.73, 138.37, 135.40, 132.84, 131.68, 131.44, 131.12, 126.57, 125.50, 122.79, 119.49, 115.34, 114.72, 113.22, 112.14, 112.02, 111.13, 94.48, 81.17, 56.87, 9.38, 7.51. LC-MS (ESI-QQQ): m/z 543.3 ([C₂₉H₂₁ F₃N₆O₂+H]⁺ calcd. 543.17). Purity 95.9% (RT 4.590 min).

N-(3-(4-cyclopropyl-1H-imidazol-1-yl)-5-(trifluoromethyl)phenyl)-3-(imidazo[1,2-b]pyridazin-3-ylethynyl)benzamide (40c). Compound 40c was prepared from 38a (0.1 g, 0.20 mmol) and 4-cyclopropyl-1H-imidazole (26 mg, 0.24 mmol) as depicted in scheme 8. The desired product was obtained as a pale yellow solid (20 mg, 19% yields). ¹H NMR (400 MHZ, DMSO-d₆) δ10.83 (s, 1H), 8.73 (dd, J=4.5, 1.6 Hz, 1H), 8.32-8.22 (m, 4H), 8.17 (d, J=1.4 Hz, 2H), 8.06 (ddd, J=7.8, 1.9, 1.2 Hz, 1H), 7.87 (dt, J=7.8, 1.3 Hz, 1H), 7.76 (d, J=1.5 Hz, 1H), 7.72-7.64 (m, 1H), 7.54 (d, J=1.5 Hz, 1H), 7.41 (dd, J=9.2, 4.5 Hz, 1H), 1.87 (tt, J=8.3, 5.0 Hz, 1H), 0.88-0.78 (m, 2H), 0.77-0.64 (m, 2H). ¹³C NMR (101 MHZ, DMSO-d₆) δ165.45, 145.71, 145.49, 141.58, 140.15, 139.02, 138.40, 135.35, 135.13, 134.96, 130.47, 129.97, 129.14, 126.62, 122.48, 119.66, 115.46, 114.82, 113.17, 97.78, 78.09, 9.37, 7.52, 0.58. LC-MS (ESI-QQQ): m/z 513.2 ([C₂₈H₁₉F₃N₆O+H]⁺ calcd. 513.16). Purity 92.5% (RT 4.670 min).

Docking studies: Molecular docking simulations were performed using AutoDock Vina 1.1.2. Pymol 2.3.1 was employed to analyze the docking results.⁵⁴ The crystal structures of wild-type BCR-ABL and BCR-ABL^{T315I} were taken from PDB ID 3OXZ and 3IK3, respectively. The protein structure was prepared by adding polar hydrogens, deleting water molecules and adding charges. Grid box was prepared based on the ligand sites that were defined in the crystal structure. The coordinate center of the search space for 3OXz was set to 12.110, −5.407, 15.591(x, y, z). The x, y and z dimension were set to 22, 24 and 34, respectively. Whereas, coordination center of the search space for 3IK3 was set to 6.487, 1.061, 17.621 (x, y, z) and x, y, z dimension were set to 22, 30, 26. For both the structures, a grid-point spacing 0f 0.375 Å was applied. The exhaustiveness was set to 48 and the maximum number of binding modes was set to 100. Other docking parameters were kept to the default values. Docking calculations were performed with full flexibility of the ligand inside the search space.

### Biological Characterization of Compounds:

### Cell Lines:

K562 and K562-T315I: The Leukemia cell lines K562 were purchased and maintained as recommended by ATCC (Manassas, VA, USA). Briefly, K562 cells were cultured in suspension in RPMI1640 (ThermoFisher Scientific, USA) supplemented with 10% fetal bovine serum and Pen/Strep/L-Glutamine. The K-562-T315I cell line was derived from the K562 line by CRISPR. Briefly, one million K562 cells were seeded in 6-well plates and transfected with Lipofectamine 2000 and 1 μg of CRISPR/Cas9 vector (pSpCas9(BB)-2A-GFP) incorporating the guide sequence (CTCAGTGATGATATAGAACG), and Lipofectamine RNAiMax and 4 μg of ssDNA donors (1ug of each donor, Supplementary Table 1) for each well of a 6-well plate. The cells were left to recover and proliferate before being selected using 1 μM imatinib in RPMI supplemented with 10% FBS. When an enriched T315I polyclonal line was achieved, imatinib selection was stopped.

iPSC-CMs: Human fibroblasts were reprogrammed to induced pluripotent stem cells (iPSCs) using Sendai viral vectors. All protocols were approved by the Stanford University Institutional Review Board. The obtained hiPSC clones were cultured in E8 cell culture media (Life Technologies) in plates coated growth factor-reduced Matrigel (Corning) until at least passage 20 before differentiation. hiPSC cells were differentiated into cardiomyocytes (CMs) utilizing a chemically defined cardiomyocyte differentiation protocol⁵⁵ and fatty acid rich maturation protocol.⁵⁶

### HAECs: Cell Viability and Growth Inhibition Assay:

Growth inhibitory activities were evaluated on K-561 leukemia cancer cell lines. The effects of the compounds on cell viability were evaluated using the AlamarBlue assay using the NCI60 methodology.⁵⁷ Cells were harvested and plated in 384-well plates (Greiner μClear) at a concentration of 1250 cells/well in 40 μL, and incubated for 24 h at 37° C. The next day, test compounds were added to the cells as a 2×40μL solution, and incubated for 48 h at 37° C. Then, the cells were treated with Resazurin (final concentration 10%) and incubated for 2 hours before measuring fluorescence on a plate reader (ex 544 nm, em 590 nm) to quantify the antiproliferative effects of the compounds.

Tube formation assay: According to the previous procedure, (ref) matrigel (vender info) was thawed overnight at 4° C. Each well of a prechilled 24-well plate was coated with 300 uL matrigel and incubated at 37° C. for 2 h. HUVEC cells (1.3×10⁵ cells) were added in 300 ul medium with compounds. After 20 h, the endothelial cell tube tube formation was assessed and imaged under an optical microscope. The tube formation numbers were counted and quantified by ImageJ software in three independent experiments.

Kinase Activity Assays: The kinase activity for ABL1 and ABL1T315I was performed using the SelectScreen™ Biochemical Kinase Profiling service of ThermoFisher Scientific (Madison, WI, USA). For each kinase, an IC₅₀ was calculated based on a 10 point concentration curve of the test article and converted to Ki values.

### Cardiotoxicity Assays:

hiPSC-CMs were plated on Matrigel coated 384-well plates at 20,000 cells per well (Greiner μClear) in 50 μl cardiomyocyte media (RMPI, B27) supplemented with 10% knock-out replacement serum. The subsequent day an additional 50 μl of media was added and cells were grown for a minimum of 5 days prior to analysis. Action potential kinetics and contractility was measured on the same cells sequentially. First, action potential kinetics were recorded using the protocol as established by Mckeithan et al.⁵⁸ Briefly, the cells were washed 5 times with FluoroBrite, loaded with VF2.1.CI dye for 50 min at 37° C., and washed again 5 times with FluoroBrite. Voltage time series were acquired at a frequency of 33 Hz for a duration of 10 s on the IC₂₀₀ Kinetic Imaging Platform (Vala Sciences). Then, the cells were loaded with wheat germ agglutinin-Alexa Fluor 555 conjugate (5 μg/ml; Life Technologies) for 10 minutes at 37° C., and then washed 4 times with FluoroBrite.⁵⁹ Contractile time series were acquired at a frequency of 50 Hz for a duration of 6.5 s. The resulting recordings were subsequently analyzed using Vala Sciences and custom software, action potential kinetics, action potential durations and rates,⁵⁸ or contractile activity parameters (peak divergence, area under the curve) were used as measures of cardiotoxicity.⁵⁹

### REFERENCES

1. Siegel, et al, Cancer Statistics, 2021. CA: a Cancer Journal for Clinicians 2021, 71 (1), 7-33.
2. Rowley, A new consistent chromosomal abnormality in chronic myelogenous leukaemia identified by quinacrine fluorescence and Giemsa staining. Nature 1973, 243 (5405), 290-293.
3. Daley, et al, Induction of chronic myelogenous leukemia in mice by the P210bcr/abl gene of the Philadelphia chromosome. Science 1990, 247 (4944), 824-830.
4. Faderl, et al, Philadelphia chromosome-positive acute lymphoblastic leukemia-current concepts and future perspectives. Reviews in clinical and experimental hematology 2002, 6 (2), 142-160.
5. Ben-Neriah, et al, The chronic myelogenous leukemia-specific P210 protein is the product of the bcr/abl hybrid gene. Science 1986, 233 (4760), 212-214.
6. Shtivelman, et al, Fused transcript of abl and bcr genes in chronic myelogenous leukaemia. Nature 1985, 315 (6020), 550-554.
7. Druker, et al, Five-year follow-up of patients receiving imatinib for chronic myeloid leukemia. New England Journal of Medicine 2006, 355 (23), 2408-2417.
8. Druker, et al, Effects of a selective inhibitor of the Abl tyrosine kinase on the growth of Bcr-Abl positive cells. Nature medicine 1996, 2 (5), 561-566.
9. Capdeville, et al, Glivec (STI571, imatinib), a rationally developed, targeted anticancer drug. Nature reviews Drug discovery 2002, 1 (7), 493-502.
10. Cohen, P., Protein kinases—the major drug targets of the twenty-first century? Nature reviews Drug discovery 2002, 1 (4), 309-315.
11. Deininger, et al, International randomized study of interferon vs STI571 (IRIS) 8-year follow up: sustained survival and low risk for progression or events in patients with newly diagnosed chronic myeloid leukemia in chronic phase (CML-CP) treated with imatinib. Blood 2009, 114 (22), 1126.
12. Hochhaus, et al, Six-year follow-up of patients receiving imatinib for the first-line treatment of chronic myeloid leukemia. Leukemia 2009, 23 (6), 1054-1061.
13. Pophali, P. A.; Patnaik, M. M., The role of new tyrosine kinase inhibitors in chronic myeloid leukemia. Cancer journal (Sudbury, Mass.) 2016, 22 (1), 40.
14. Hughes, et al, Monitoring CML patients responding to treatment with tyrosine kinase inhibitors: review and recommendations for harmonizing current methodology for detecting BCR-ABL transcripts and kinase domain mutations and for expressing results. Blood 2006, 108 (1), 28-37.
15. O'Hare, et al, Bcr-Abl kinase domain mutations, drug resistance, and the road to a cure for chronic myeloid leukemia. Blood 2007, 110 (7), 2242-2249.
16. Shah, et al, Overriding imatinib resistance with a novel ABL kinase inhibitor. Science 2004, 305 (5682), 399-401.
17. Weisberg, et al, Characterization of AMN107, a selective inhibitor of native and mutant Bcr-Abl. Cancer cell 2005, 7 (2), 129-141.
18. Saglio, et al, Nilotinib versus imatinib for newly diagnosed chronic myeloid leukemia. New England Journal of Medicine 2010, 362 (24), 2251-2259.
19. Kantarjian, et al; Dasatinib versus imatinib in newly diagnosed chronic-phase chronic myeloid leukemia. New England Journal of Medicine 2010, 362 (24), 2260-2270.
20. Vener, et al, First-line imatinib vs second-and third-generation TKIs for chronic-phase CML: a systematic review and meta-analysis. Blood advances 2020, 4 (12), 2723-2735.
21. Quintás-Cardama, et al, Therapeutic options against BCR-ABL1 T315I-positive chronic myelogenous leukemia. Clinical Cancer Research 2008, 14 (14), 4392-4399.
22. O'Hare, et al, In vitro activity of Bcr-Abl inhibitors AMN107 and BMS-354825 against clinically relevant imatinib-resistant Abl kinase domain mutants. Cancer research 2005, 65 (11), 4500-4505.
23. Miller, et al, Resistant mutations in CML and Ph(+)ALL—role of ponatinib. Biologics 2014, 8, 243-54.
24. Tokarski, et al, The structure of Dasatinib (BMS-354825) bound to activated ABL kinase domain elucidates its inhibitory activity against imatinib-resistant ABL mutants. Cancer research 2006, 66 (11), 5790-5797.
25. Hughes, et al, Long-term outcomes in patients with chronic myeloid leukemia in chronic phase receiving frontline nilotinib versus imatinib: ENESTnd 10-year analysis. American Society of Hematology Washington, DC: 2019; pp 2924-2924.
26. Jabbour, et al, Chronic myeloid leukemia: 2020 update on diagnosis, therapy and monitoring. Am J Hemato/ 2020, 95 (6), 691-709.
27. Giles, et al, MK-0457, a novel kinase inhibitor, is active in patients with chronic myeloid leukemia or acute lymphocytic leukemia with the T315I BCR-ABL mutation. Blood 2007, 109 (2), 500-502.
28. FDA approves Ponatinib. fda.gov/drugs/informationondrugs/approveddrugs/ucm381452.htm.
29. O'Hare, et al, AP24534, a pan-BCR-ABL inhibitor for chronic myeloid leukemia, potently inhibits the T315I mutant and overcomes mutation-based resistance. Cancer Cell 2009, 16 (5), 401-12.
30. Cortes, et al; Investigators, P., A phase 2 trial of ponatinib in Philadelphia chromosome-positive leukemias. N Engl J Med 2013, 369 (19), 1783-96.
31. Cortes, et al, Long-term follow-up of ponatinib efficacy and safety in the phase 2 PACE trial. American Society of Hematology Washington, DC: 2014.
32. Gainor, et al, Ponatinib: accelerated disapproval. The oncologist 2015, 20 (8), 847.
33. Singh, et al, Cardiotoxicity of the BCR-ABL1 tyrosine kinase inhibitors: Emphasis on ponatinib. Int J Cardiol 2020, 316, 214-221.
34. Moslehi, et al, Tyrosine Kinase Inhibitor-Associated Cardiovascular Toxicity in Chronic Myeloid Leukemia. J Clin Oncol 2015, 33 (35), 4210-8.
35. Ishoey, et al, Translation termination factor GSPT1 is a phenotypically relevant off-target of heterobifunctional phthalimide degraders. ACS chemical biology 2018, 13 (3), 553-560.
36. Latifi, et al, Thrombotic microangiopathy as a cause of cardiovascular toxicity from the BCR-ABL1 tyrosine kinase inhibitor ponatinib. Blood 2019, 133 (14), 1597-1606.
37. Yang, et al, Global PROTAC Toolbox for Degrading BCR-ABL Overcomes Drug-Resistant Mutants and Adverse Effects. J Med Chem 2020, (63), 8567-8585.
38. Larocque, et al, Nicotinamide-Ponatinib Analogues as Potent Anti-CML and Anti-AML Compounds. ACS Omega 2020, 5 (6), 2690-2698.
39. Nagar, et al, Crystal structures of the kinase domain of c-Abl in complex with the small molecule inhibitors PD173955 and imatinib (STI-571). Cancer research 2002, 62 (15), 4236-4243.
40. Levinson, et al, Structural and spectroscopic analysis of the kinase inhibitor bosutinib and an isomer of bosutinib binding to the Abl tyrosine kinase domain. PloS one 2012, 7 (4), e29828.
41. Zhou, et al, Structural mechanism of the Pan-BCR-ABL inhibitor ponatinib (AP24534): lessons for overcoming kinase inhibitor resistance. Chem Biol Drug Des 2011, 77 (1), 1-11.
42. Chen, et al, Discovery of novel 2-(aminoheteroaryl)-thiazole-5-carboxamides as potent and orally active Src-family kinase p56Lck inhibitors. Bioorganic & medicinal chemistry letters 2004, 14 (24), 6061-6066.
43. Huang, et al, Discovery of 3-[2-(imidazo[1,2-b]pyridazin-3-yl)ethynyl]-4-methyl-N-{4-[(4-methylpiperazin-1-yl)methyl]-3-(trifluoromethyl)phenyl}benzamide (AP24534), a potent, orally active pan-inhibitor of breakpoint cluster region-abelson (BCR-ABL) kinase including the T315I gatekeeper mutant. J Med Chem 2010, 53 (12), 4701-19.
44. Huang, et al, An efficient synthesis of nilotinib (AMN107). Synthesis 2007, 2007 (14), 2121-2124.
45. Wen, et al, An efficient D-glucosamine-based copper catalyst for C-X couplings and its application in the synthesis of nilotinib intermediate. RSC advances 2015, 5 (2), 1522-1528.
46. Bhunia, et al, Selected Copper-Based Reactions for C—N, C—O, C—S, and C—C Bond Formation. Angewandte Chemie International Edition 2017, 56 (51), 16136-16179.
47. Zimmermann, et al, Potent and selective inhibitors of the Abl-kinase: phenylamino-pyrimidine (PAP) derivatives. Bioorganic & Medicinal Chemistry Letters 1997, 7 (2), 187-192.
48. Li, et al, Design, synthesis, and biological evaluation of 3-(1 H-1, 2, 3-triazol-1-yl) benzamide derivatives as potent pan Bcr-Abl inhibitors including the threonine315→isoleucine315 mutant. Journal of medicinal chemistry 2012, 55 (22), 10033-10046.
49. Wang, et al, Design, Synthesis, and Biological Evaluation of 3-(Imidazo [1, 2-a] pyrazin-3-ylethynyl)-4-isopropyl-N-(3-((4-methylpiperazin-1-yl) methyl)-5-(trifluoromethyl)phenyl) benzamide as a Dual Inhibitor of Discoidin Domain Receptors 1 and 2. Journal of medicinal chemistry 2018, 61 (17), 7977-7990.
50. Rix, et al, Chemical proteomic profiles of the BCR-ABL inhibitors imatinib, nilotinib, and dasatinib reveal novel kinase and nonkinase targets. Blood 2007, 110 (12), 4055-4063.
51. Singh, et al, Ponatinib-induced cardiotoxicity: delineating the signalling mechanisms and potential rescue strategies. Cardiovascular research 2019, 115 (5), 966-977.
52. Jin, et al, A comprehensive review of clinical cardiotoxicity incidence of FDA-approved small-molecule kinase inhibitors. Frontiers in Pharmacology 2020, 11, 891.
53. Najjar, et al, Structure guided design of potent and selective ponatinib-based hybrid inhibitors for RIPK1. Cell reports 2015, 10 (11), 1850-1860.
54. Trott, et al: improving the speed and accuracy of docking with a new scoring function, efficient optimization, and multithreading. Journal of computational chemistry 2010, 31 (2), 455-461.
55. Lian, et al, Robust cardiomyocyte differentiation from human pluripotent stem cells via temporal modulation of canonical Wnt signaling. Proceedings of the National Academy of Sciences 2012, 109 (27), E1848-E1857.
56. Feyen, et al, Metabolic maturation media improve physiological function of human iPSC-derived cardiomyocytes. Cell reports 2020, 32 (3), 107925.
57. Shoemaker, R. H., The NCI60 human tumour cell line anticancer drug screen. Nature Reviews Cancer 2006, 6 (10), 813-823.
58. McKeithan, et al, An automated platform for assessment of congenital and drug-induced arrhythmia with hiPSC-derived cardiomyocytes. Frontiers in physiology 2017, 8, 766.
59. Sharma, et al., High-throughput screening of tyrosine kinase inhibitor cardiotoxicity with human induced pluripotent stem cells. Science translational medicine 2017, 9 (377).

What is claimed:

1. A compound of Formula (I): 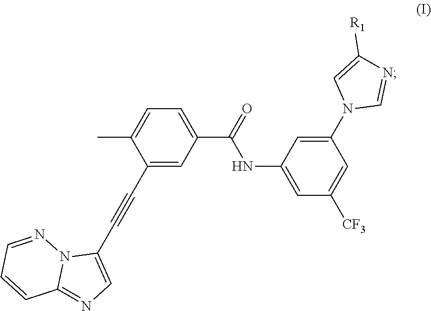
wherein R₁ is selected from the group of H, C₂-C₆ alkyl, C₃-C₆ cycloalkyl, and —CH₂-C₃-C₆ cycloalkyl; or a pharmaceutically acceptable salt thereof.

2. The compound of claim 1, wherein R₁ is selected from the group of H, ethyl, n-propyl, isopropyl and cyclopropyl; or a pharmaceutically acceptable salt thereof.

3. The compound of any of claims 1 and 2, wherein R₁ is selected from the group of H, ethyl, isopropyl and cyclopropyl; or a pharmaceutically acceptable salt thereof.

4. The compound of any of claims 1, 2, and 3, or a pharmaceutically acceptable salt thereof, wherein R₁ is selected from the group of H, ethyl, and cyclopropyl.

5. The compound of any of claims 1, 2, 3, and 4, or a pharmaceutically acceptable salt thereof, wherein R₁ is selected from the group of H and ethyl.

6. The compound of any of claims 1, 2, 3, 4, and 5, or a pharmaceutically acceptable salt thereof, wherein R₁ is selected from the group of H and isopropyl.

7. The compound of any of claims 1, 2, 3, 4, 5, and 6, or a pharmaceutically acceptable salt thereof, wherein R₁ is selected from the group of H and cyclopropyl.

8. The compound of any of claims 1, 2, 3, 4, 5, 6, and 7, or a pharmaceutically acceptable salt thereof, having the structure:

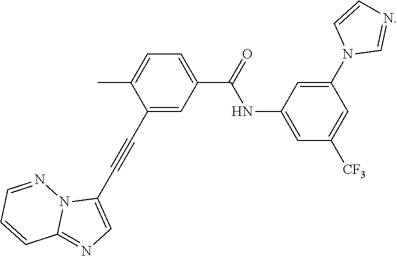 [SMILES: Cc1ccc(C(=O)Nc2cc(-n3ccnc3)cc(C(F)(F)F)c2)cc1C#Cc1cnc2cccnn12]

9. The compound of any of claims 1, 2, 3, 4, and 5, or a pharmaceutically acceptable salt thereof, having the structure:

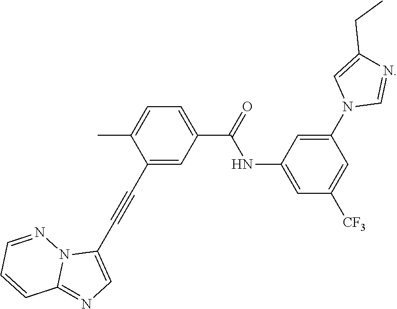 [SMILES: CCc1cn(-c2cc(NC(=O)c3ccc(C)c(C#Cc4cnc5cccnn45)c3)cc(C(F)(F)F)c2)cn1]

10. The compound of any of claims 1, 2, 3, and 6, or a pharmaceutically acceptable salt thereof, having the structure:

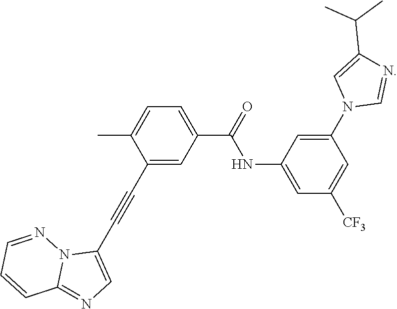 [SMILES: Cc1ccc(C(=O)Nc2cc(-n3cnc(C(C)C)c3)cc(C(F)(F)F)c2)cc1C#Cc1cnc2cccnn12]

11. The compound of any of claims 1, 2, 3, and 4, or a pharmaceutically acceptable salt thereof, having the structure:

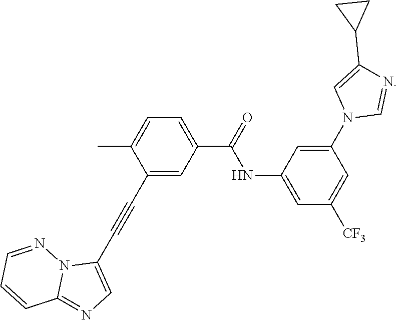 [SMILES: Cc1ccc(C(=O)Nc2cc(-n3cnc(C4CC4)c3)cc(C(F)(F)F)c2)cc1C#Cc1cnc2cccnn12]

12. A compound of Formula (II): 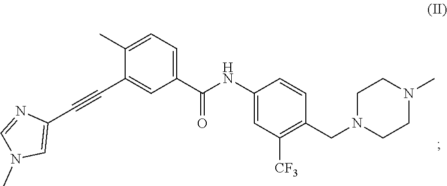 [SMILES: Cc1ccc(C(=O)Nc2ccc(CN3CCN(C)CC3)c(C(F)(F)F)c2)cc1C#Cc1cn(C)cn1]
or a pharmaceutically acceptable salt thereof.

13. A method of treatment of chronic myeloid leukemia in a subject, the method comprising administering to the subject in need thereof a pharmaceutically effective amount of a compound of any of claims 1-11 (claims 1, 2, 3, 4, 5, 6, 7, 8, 9, 10, and 11), or a pharmaceutically acceptable salt thereof.

14. A method of treatment of chronic myeloid leukemia in a subject, the method comprising administering to the subject in need thereof a pharmaceutically effective amount of a compound of claim 12, or a pharmaceutically acceptable salt thereof.

15. A method of inhibiting the activity of the BCR-ABL kinase protein in a subject, the method comprising administering to the subject in need thereof a pharmaceutically effective amount of a compound of any of claims 1-11, or a pharmaceutically acceptable salt thereof.

16. A method of inhibiting the activity of the BCR-ABL kinase protein in a subject, the method comprising administering to the subject in need thereof a pharmaceutically effective amount of a compound of claim 12, or a pharmaceutically acceptable salt thereof.

17. A method of treatment of chronic myeloid leukemia in a subject, the method comprising administering to the subject in need thereof:
a) a pharmaceutically effective amount of a compound of any of claims 1-11, or a pharmaceutically acceptable salt thereof; and
b) a pharmaceutically effective amount of one or more agents selected from the group of ponatinib, nilotinib, imatinib, dasatinib, bosutinib, rebastinib, and interferon alfa-2b; or a pharmaceutically acceptable salt thereof.

18. A method of treatment of chronic myeloid leukemia in a subject, the method comprising administering to the subject in need thereof:
a) a pharmaceutically effective amount of a compound of claim 12, or a pharmaceutically acceptable salt thereof; and
b) a pharmaceutically effective amount of one or more agents selected from the group of ponatinib, nilotinib, imatinib, dasatinib, bosutinib, rebastinib, and interferon alfa-2b; or a pharmaceutically acceptable salt thereof.

19. A method of treatment for chronic phase chronic myeloid leukemia in a subject, the method comprising administering to the subject in need thereof a pharmaceutically effective amount of a compound of any of claims 1-11, or a pharmaceutically acceptable salt thereof.

20. A method of treatment for chronic phase chronic myeloid leukemia in a subject, the method comprising administering to the subject in need thereof a pharmaceutically effective amount of a compound of claim 12, or a pharmaceutically acceptable salt thereof.

21. A method of treatment of chronic phase chronic myeloid leukemia in a subject, the method comprising administering to the subject in need thereof:
a) a pharmaceutically effective amount of a compound of any of claims 1-11, or a pharmaceutically acceptable salt thereof; and
b) a pharmaceutically effective amount of one or more tyrosine kinase inhibiting agents selected from the group of ponatinib, nilotinib, imatinib, dasatinib, bosutinib, and rebastinib; or a pharmaceutically acceptable salt thereof.

22. A method of treatment of chronic phase chronic myeloid leukemia in a subject, the method comprising administering to the subject in need thereof:
a) a pharmaceutically effective amount of a compound of claim 12, or a pharmaceutically acceptable salt thereof; and
b) a pharmaceutically effective amount of one or more tyrosine kinase inhibiting agents selected from the group of ponatinib, nilotinib, imatinib, dasatinib, bosutinib, and rebastinib; or a pharmaceutically acceptable salt thereof.

23. A method of treatment in a subject of chronic phase chronic myeloid leukemia with resistance or intolerance to at least one prior tyrosine kinase inhibitor, the method comprising administering to the subject in need thereof:
a) a pharmaceutically effective amount of a compound of any of claims 1-11, or a pharmaceutically acceptable salt thereof; and
b) a pharmaceutically effective amount of ponatinib; or a pharmaceutically acceptable salt thereof.

24. A method of treatment in a subject of chronic phase chronic myeloid leukemia with resistance or intolerance to at least one prior tyrosine kinase inhibitor, the method comprising administering to the subject in need thereof:
a) a pharmaceutically effective amount of a compound of claim 12, or a pharmaceutically acceptable salt thereof; and
b) a pharmaceutically effective amount of ponatinib; or a pharmaceutically acceptable salt thereof.

25. A method of treatment in a subject of chronic phase chronic myeloid leukemia with resistance or intolerance to at least two prior tyrosine kinase inhibitors, the method comprising administering to the subject in need thereof:
a) a pharmaceutically effective amount of a compound of any of claims 1-11, or a pharmaceutically acceptable salt thereof; and
b) a pharmaceutically effective amount of ponatinib; or a pharmaceutically acceptable salt thereof.

26. A method of treatment in a subject of chronic phase chronic myeloid leukemia with resistance or intolerance to at least two prior tyrosine kinase inhibitors, the method comprising administering to the subject in need thereof:
a) a pharmaceutically effective amount of a compound of claim 12, or a pharmaceutically acceptable salt thereof; and
b) a pharmaceutically effective amount of ponatinib; or a pharmaceutically acceptable salt thereof.

27. A method of treatment of accelerated phase chronic myeloid leukemia in a subject, the method comprising administering to the subject in need thereof:
a) a pharmaceutically effective amount of a compound of any of claims 1-11, or a pharmaceutically acceptable salt thereof; and
b) a pharmaceutically effective amount of one or more tyrosine kinase inhibiting agents selected from the group of ponatinib, nilotinib, imatinib, dasatinib, bosutinib, and rebastinib; or a pharmaceutically acceptable salt thereof.

28. A method of treatment of accelerated phase chronic myeloid leukemia in a subject, the method comprising administering to the subject in need thereof:
a) a pharmaceutically effective amount of a compound of claim 12, or a pharmaceutically acceptable salt thereof; and
b) a pharmaceutically effective amount of one or more tyrosine kinase inhibiting agents selected from the group of ponatinib, nilotinib, imatinib, dasatinib, bosutinib, and rebastinib; or a pharmaceutically acceptable salt thereof.

29. A method of treatment of blast phase chronic myeloid leukemia in a subject, the method comprising administering to the subject in need thereof:
a) a pharmaceutically effective amount of a compound of any of claims 1-11, or a pharmaceutically acceptable salt thereof; and
b) a pharmaceutically effective amount of one or more tyrosine kinase inhibiting agents selected from the group of ponatinib, nilotinib, imatinib, dasatinib, bosutinib, and rebastinib; or a pharmaceutically acceptable salt thereof.

30. A method of treatment of blast phase chronic myeloid leukemia in a subject, the method comprising administering to the subject in need thereof:
a) a pharmaceutically effective amount of a compound of claim 12, or a pharmaceutically acceptable salt thereof; and
b) a pharmaceutically effective amount of one or more tyrosine kinase inhibiting agents selected from the group of ponatinib (ICLUSIG®), nilotinib (TASIGNA®), imatinib (GLEEVEC®), dasatinib (SPRYCELL®), bosutinib (BOSULIF®), and rebastinib; or a pharmaceutically acceptable salt thereof.

31. A method of treatment of chronic myeloid leukemia with a T315l mutation in a subject, the method comprising administering to the subject in need thereof:
a) a pharmaceutically effective amount of a compound of any of claims 1-11, or a pharmaceutically acceptable salt thereof; and
b) a pharmaceutically effective amount of omacetaxine (SYNRIBO®); or a pharmaceutically acceptable salt thereof.

32. A method of treatment of chronic myeloid leukemia with a T315l mutation in a subject, the method comprising administering to the subject in need thereof:
a) a pharmaceutically effective amount of a compound of claim 12, or a pharmaceutically acceptable salt thereof; and
b) a pharmaceutically effective amount of omacetaxine (SYNRIBO®); or a pharmaceutically acceptable salt thereof.

33. A method of treatment of Philadelphia chromosome positive chronic myeloid leukemia in a subject, the method comprising administering to the subject in need thereof:
a) a pharmaceutically effective amount of a compound of any of claims 1-11, or a pharmaceutically acceptable salt thereof; and
b) a pharmaceutically effective amount of nilotinib (TASIGNA®); or a pharmaceutically acceptable salt thereof.

34. A method of treatment of Philadelphia chromosome positive chronic myeloid leukemia in a subject, the method comprising administering to the subject in need thereof:
a) a pharmaceutically effective amount of a compound of claim 12, or a pharmaceutically acceptable salt thereof; and
b) a pharmaceutically effective amount of nilotinib (TASIGNA®); or a pharmaceutically acceptable salt thereof.

35. A method of treatment in a subject of chronic myeloid leukemia that is resistant or intolerant to prior tyrosine-kinase inhibitor (TKI) therapy, the method comprising administering to the subject in need thereof a pharmaceutically effective amount of a compound of any of claims 1-11, or a pharmaceutically acceptable salt thereof.

36. A method of treatment in a subject of chronic myeloid leukemia that is resistant or intolerant to prior tyrosine-kinase inhibitor (TKI) therapy, the method comprising administering to the subject in need thereof a pharmaceutically effective amount of a compound of claim 12, or a pharmaceutically acceptable salt thereof.

37. A method of treating a neurodegenerative condition in a subject, the method comprising administering to the subject in need thereof a pharmaceutically effective amount of a compound of any of claims 1-11, or a pharmaceutically acceptable salt thereof.

38. A method of treating a neurodegenerative condition in a subject, the method comprising administering to the subject in need thereof a pharmaceutically effective amount of a compound of any of claim 12, or a pharmaceutically acceptable salt thereof.

39. The method of any of claims 36 and 37, wherein the neurodegenerative disease is selected from the group of Parkinson's Disease, Alzheimer's Disease, Down's syndrome, frontotemporal dementia, progressive supranuclear palsy, Pick's disease, Niemann-Pick disease, Parkinson's disease, Huntington's disease, dentatorubropallidoluysian atrophy, Kennedy's disease (also referred to as spinobulbar muscular atrophy), and spinocerebellar ataxia (e.g., type I, type 2, type 3 (also referred to as Machado-Joseph disease), type 6, type 7, and type 17)), fragile X (Rett's) syndrome, fragile XE mental retardation, Friedreich's ataxia, myotonic dystrophy, spinocerebellar ataxia type 8, and spinocerebellar ataxia type 12, Alexander disease, Alper's disease, amyotrophic lateral sclerosis, ataxia telangiectasia, Batten disease (also referred to as Spielmeyer-Vogt-Sjogren-Batten disease), Canavan disease, Cockayne syndrome, corticobasal degeneration, Creutzfeldt-Jakob disease, ischemia stroke, Krabbe disease, Lewy body dementia, multiple sclerosis, multiple system atrophy, Pelizaeus-Merzbacher disease, Pick's disease, primary lateral sclerosis, Adult Refsums Disease (ARD), Sandhoff disease, Schilder's disease, spinal cord injury, spinal muscular atrophy, Steele-Richardson-Olszewski disease, and Tabes dorsalis.

40. The method of any of claims 37, 38, and 39, wherein the neurodegenerative condition is associated with, characterized by, or implicated by a mitochondrial dysfunction.

41. The method of claim 40, wherein the neurodegenerative condition is selected from the group of Friedrich's ataxia, amyotrophic lateral sclerosis (ALS), mitochondrial myopathy, encephalopathy, lactacidosis, stroke (MELAS), myoclonic epilepsy with ragged red fibers (MERFF), epilepsy, Parkinson's disease, Alzheimer's disease, and Huntington's Disease.

42. A pharmaceutical composition comprising a pharmaceutically effective amount of a compound of any of claims 1-11, or a pharmaceutically acceptable salt thereof, and a pharmaceutically useful carrier or excipient.

43. A pharmaceutical composition comprising a pharmaceutically effective amount of a compound of claim 12, or a pharmaceutically acceptable salt thereof, and a pharmaceutically useful carrier or excipient.

44. The use of a compound selected from any of claims 1-11, or a pharmaceutically acceptable salt thereof, in the preparation of a medicament.

45. The use of a compound selected from any of claims 1-11, or a pharmaceutically acceptable salt thereof, in the preparation of a medicament for use in a method of treatment selected from those of any of claims 13, 15, 17, 19, 21, 23, 25, 27, 29, 31, 33, 35, 37, 39, 41, and 42.

46. The use of a compound of claim 12, or a pharmaceutically acceptable salt thereof, in the preparation of a medicament.

47. The use of a compound selected from claim 12, or a pharmaceutically acceptable salt thereof, in the preparation of a medicament for use in a method of treatment selected from those of any of claims 14, 16, 18, 20, 22, 24, 26, 28, 30, 32, 34, 36, 38, 39, 41, and 42.

48. A compound of Formula (III), wherein R₁ is selected from the group of H and —OCH₃: 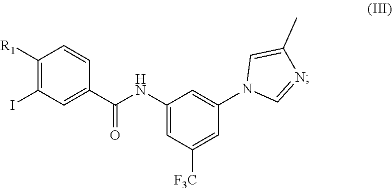
or a pharmaceutically acceptable salt thereof.
